(19) United States
(12) Patent Application Publication
(10) Pub. No.: US 2024/0131155 A1
(43) Pub. Date: Apr. 25, 2024

(54) T CELLS FOR USE IN THERAPY

(71) Applicant: Erasmus University Medical Center Rotterdam, Rotterdam (NL)

(72) Inventors: Johannes Eduard Maria Antonius DEBETS, Rotterdam (NL); Dora Martha HAMMERL, Rotterdam (NL)

(21) Appl. No.: 18/273,468

(22) PCT Filed: Jan. 21, 2022
(86) PCT No.: PCT/NL2022/050028

§ 371 Date: Jul. 20, 2023

(30) Foreign Application Priority Data
Jan. 21, 2021 (EP) 21152822.9

(51) Int. Cl. A61K 39/00; A61P 35/00; C07K 14/725; C07K 16/28

(52) U.S. Cl. CPC A61K 39/4611; A61K 39/4632; A61K 39/464414; A61P 35/00; C07K 14/7051; C07K 16/28; A61K 2239/49; C07K 2319/02; C07K 2319/03

(57) ABSTRACT

The invention provides inter alia an engineered T cell, wherein said T cell is engineered to express a T cell receptor (TCR) or an antibody-based receptor that binds to a T cell epitope of human ropporin-1A (ROPN1) or human ropporin-1B (ROPN1B); wherein said T cell epitope is selected from the group consisting of SEQ ID NO:4, SEQ ID NO:43, SEQ ID NO:23, SEQ ID NO:56 and SEQ ID NO:24.

### FIELD OF THE INVENTION

The invention is in the field of T cell therapy. More specifically, the invention relates to T cell epitopes of human ropporin-1A (ROPN1) and/or human ropporin-1B (ROPN1B), T cell receptors (TCRs) or antibody-based receptors with binding specificity towards ROPN1 and ROPN1B, and engineered (genetically modified) T cells that are engineered to express (forced to express) a T cell receptor or antibody-based receptor that binds to (or has binding specificity towards) an epitope of ROPN1 and/or ROPN1B. The engineered T cells can be used in immunotherapy, for instance in the treatment of a solid tumor, such as breast cancer, skin cancer, or a hematological tumor, such as myeloma or lymphoma.

### BACKGROUND TO THE INVENTION

Adoptive T cell therapies (AT) generally rely on isolation of T cells from patients' blood, insertion of genes encoding either for a chimeric antigen receptor (CAR) or a TCR with pre-defined antigen specificity, expansion of these cells, and re-infusion of the engineered, autologous T cell product into the patient. This strategy has been applied to different tumor types with variable successes, mainly depending on type of tumor, target antigen and receptor (Debets et al., Semin Immunol. 28(1):10-21 (2016), doi:10.1016/j.smim.2016.03.002; Johnson et al., Cell Res. 27(1):38-58 (2017), doi:10.1038/cr.2016.154; and Sadelain et al., Nature. 545(7655):423-431 (2017), doi:10.1038/nature22395).

Treatment with CAR T cells is considered a breakthrough for B-cell malignancies (objective response rate (OR): 95%), and CD19-directed CAR T cell products (i.e., Kymriah, Yescarta, Tecartus) have recently been approved by the FDA and EMA to treat these malignancies. Unfortunately, the efficacy of CAR T cells to treat solid tumors is significantly lagging behind the efficacy observed for hematological malignancies. It is noteworthy that CARs recognize extracellular targets (i.e., covering about 30% of all targets), whereas TCRs recognize both extra- and intracellular targets (i.e., covering 100% of all targets). Indeed, in some instances TCR-engineered T cells have revealed clear clinical responses when used to treat solid as well as blood tumor types (Kunert et al., Front Immunol. 4(November):1-16 (2013), doi:10.3389/fimmu.2013.00363; and Johnson et al., Cell Res. 27(1):38-58 (2017), doi:10.1038/cr.2016.154). For example, in melanoma, synovial sarcoma and multiple myeloma ORs of 55%, 61% and 80%, respectively, have been observed for AT with T cells expressing a NY-ES01-specific TCR (Robbins et al., Clin Can Res doi: 10.1158/1078-0432, Rapoport et al., Nat Med.21(8):914-921 (2015), doi:10.1038/nm.3910).

Despite some clinical successes, a major challenge for the treatment with engineered T cells, whether it be CAR or TCR T cells, is preventing treatment-related toxicities. Such toxicities include on-target toxicities (i.e., engineered T cells recognizing identical targets outside tumor tissue) as well as off-target toxicities (i.e., engineered T cells recognizing targets that are highly similar to their cognate targets outside tumor tissue) (Debets et al., Semin Immuno1.28(1):10-21 (2016), doi:10.1016/j.smim.2016.03.002). Treatment-related toxicities generally depend on the choice of the target antigen and TCR. For instance, a CAR targeting the CAIX antigen resulted in severe on-target toxicity (Lamers et Mol Ther.21(4):904-912 (2013), doi:10.1038/mt.2013.17), and affinity-enhanced TCRs targeting the MAGE-A3 antigen were accompanied by severe off-target toxicities (Cameron et al., Sci Transl Med.5(197):197ra103-197ra103 (2013), doi:10.1126/scitranslmed.3006034; and Morgan et al., J Immunother.36(2):133-151 (2014), doi:10.1097/CJI.0b013e3182829903.Cancer).

Another challenge, particularly for the treatment of solid tumors, is heterogeneic expression of target antigens in tumor tissues, ranging from few to many tumor cells, which may limit the efficacy of AT (Majzner et al., Cancer Discov.8(10):1219-1226 (2018), doi:10.1158/2159-8290.CD-18-0442).

A final challenge, also related to the treatment of solid tumors, is the current lack of targets that enable treatment of large cohorts of patients, which is attributable to under-developed research into intracellular antigens.

Taken the above challenges together, there is a clear need in the art to identify and exploit tumor-selective and immunogenic target antigens, their epitopes and corresponding TCRs. It is of imperative importance to select targets, epitopes and TCRs in such a way to avoid treatment-related toxicities, yet at the same time guarantee T cell responses against immunogenic and homogenously expressed targets and epitopes that enable treatment of large cohorts of cancer patients.

An aim of the present invention is to provide tumor-selective and immunogenic T cell epitopes that are derived from a target antigen that is homogenously and frequently expressed in certain cancer types, and to target such epitopes with T cells engineered to express a TCR that has strict epitope specificity (i.e., being not cross-reactive against other, highly similar epitopes).

### SUMMARY OF THE INVENTION

The invention provides an engineered T cell, wherein said T cell is engineered to express a T cell receptor (TCR) or an antibody-based receptor, e.g. chimeric antigen receptor (CAR), that binds to a T cell epitope of human ropporin-1A (ROPN1) and/or human ropporin-1B (ROPN1B); wherein said T cell epitope consists of the amino acid sequence selected from one of SEQ ID NO:4, SEQ ID NO:43, SEQ ID NO:23, SEQ ID NO:56 and SEQ ID NO:24.

The invention also provides an engineered T cell, wherein said T cell is engineered to express a T cell receptor (TCR), or an antibody-based receptor (such as a chimeric antigen receptor (CAR), that binds to a T cell epitope of human ropporin-1B (ROPN1B) and/or human ropporin-1A (ROPN1), such as one or more of epitopes 1-11, preferably epitopes 4 (FLY-A epitope), 10 (FLY-B epitope) or 11 (EVI epitope), more preferably epitope 4 (FLY-A).

The inventors have identified human ropporin-1A (ROPN1) and ropporin-1B (ROPN1B) as T-cell target antigens that have a tumor-restricted expression, which has a high and homogenous expression in both breast cancer, such as triple-negative breast cancer (TNBC), and skin cancer, such as skin cutaneous melanoma (SKCM) (see Example 1 and FIGS. 1 and 2), but also in certain hematological malignancies, such as myelomas (e.g. multiple myeloma (MM)). The inventors identified a set of eleven human ROPN1 and ROPN1B T cell epitopes that are tumor-selective and safe (i.e., not part of any protein other than ROPN1 and ROPN1B) (Example 1, FIG. 3, Table 1), which after further screening was reduced to a set of nine human ROPN1 and ROPN1B T cell epitopes. These ROPN1 and ROPN1B epitopes are highly immunogenic as is evidenced by T cell responses raised against said epitopes (Table 2). Further, the inventors isolated a TCR that binds to an epitope of ROPN1B, SEQ ID NO:1 (MLN epitope (epitope 1)), and the sequences of the TCR alpha and beta chains were determined (Example 1, FIG. 4, and SEQ ID NOs:10-19, 21, 22). It was further established that this TCR is functional and specifically recognizes the MLN epitope when engineered into a T cell (Example 1, FIG. 5).

The inventors also identified two further ROPN1B epitopes (SEQ ID NO: 23 (also referred to as ‘FLY-B epitope’ or ‘epitope 10’) and SEQ ID NO: 24 (also referred to as ‘EVI epitope’ or ‘epitope 11’)). The inventors have further identified T cell receptors (TCRs) that bind to epitopes 4 (SEQ ID NO:4, also referred to as ‘epitope 4’ or ‘FLY-A epitope’), 10 and 11. These TCRs, when transduced into a T cell, provide a gene-engineered T cell that results in a sensitive and specific recognition of the cognate epitope and results in effective tumor cell killing (Example 2, FIGS. 7+). T cells that are gene-engineered to express a TCR (trans)gene that either binds to epitopes 4 (FLY-A), 10 (FLY-B) and 11 (EVI), and the TCRs as such, are highly preferred embodiments of the present invention.

In a preferred embodiment of an engineered T cell of the invention, said T cell is engineered to express a TCR that binds to a T cell epitope of SEQ ID NO:4 and/or SEQ ID NO:43; and wherein said TCR comprises: (i) a T cell receptor alpha chain comprising a hypervariable region that comprises a CDR3 of SEQ ID NO:37, and (ii) a T cell receptor beta chain comprising a hypervariable region that comprises a CDR3 of SEQ ID NO:42; and wherein said hypervariable regions of said T cell receptor alpha and beta chain further comprise a CDR1 and a CDR2.

In another preferred embodiment of an engineered T cell of the invention, said hypervariable region of said TCR alpha chain comprises: a CDR1 of SEQ ID NO:35;—a CDR2 of SEQ ID NO:36;—a CDR3 of SEQ ID NO:37; and wherein said hypervariable region of said T cell receptor beta chain comprises:—a CDR1 of SEQ ID NO:40;—a CDR2 of SEQ ID NO:41;—a CDR3 of SEQ ID NO:42.

In another preferred embodiment of an engineered T cell of the invention, said T cell receptor alpha chain comprises an amino acid sequence of SEQ ID NO:44 and wherein said T cell receptor beta chain comprises an amino acid sequence of SEQ ID NO:45; preferably wherein said T cell receptor alpha chain comprises an amino acid sequence of SEQ ID NO:34 (optionally without the leader sequence as shown in FIG. 14 or with an alternative leader sequence) and wherein said T cell receptor beta chain comprises an amino acid sequence of SEQ ID NO:39 (optionally without the leader sequence as shown in FIG. 14 or with an alternative leader sequence).

In an alternative embodiment, said T cell is engineered to express a TCR that binds to a T cell epitope of SEQ ID NO:23 and/or SEQ ID NO:56; and wherein said TCR comprises: (i) a T cell receptor alpha chain comprising a hypervariable region that comprises a CDR3 of SEQ ID NO:50, and (ii) a T cell receptor beta chain comprising a hypervariable region that comprises a CDR3 of SEQ ID NO:55; and wherein said hypervariable regions of said T cell receptor alpha and beta chain further comprise a CDR1 and a CDR2.

In a preferred embodiment of said engineered T cell of the invention, said hypervariable region of said TCR alpha chain comprises: a CDR1 of SEQ ID NO:48;—a CDR2 of SEQ ID NO:49;—a CDR3 of SEQ ID NO:50; and wherein said hypervariable region of said T cell receptor beta chain comprises:—a CDR1 of SEQ ID NO:53;—a CDR2 of SEQ ID NO:54;—a CDR3 of SEQ ID NO:55.

In another preferred embodiment of said engineered T cell of the invention, said T cell receptor alpha chain comprises an amino acid sequence of SEQ ID NO:57 and wherein said T cell receptor beta chain comprises an amino acid sequence of SEQ ID NO:58; preferably wherein said T cell receptor alpha chain comprises an amino acid sequence of SEQ ID NO:47 (optionally without the leader sequence as shown in FIG. 14 or with an alternative leader sequence) and wherein said T cell receptor beta chain comprises an amino acid sequence of SEQ ID NO:52 (optionally without the leader sequence as shown in FIG. 14 or with an alternative leader sequence).

In another alternative embodiment, said T cell is engineered to express a TCR that binds to a T cell epitope of SEQ ID NO:24; and wherein said TCR comprises: (i) a T cell receptor alpha chain comprising a hypervariable region that comprises a CDR3 of SEQ ID NO:63, and (ii) a T cell receptor beta chain comprising a hypervariable region that comprises a CDR3 of SEQ ID NO:68; and wherein said hypervariable regions of said T cell receptor alpha and beta chain further comprise a CDR1 and a CDR2.

In a preferred embodiment of said engineered T cell of the invention, said hypervariable region of said TCR alpha chain comprises: a CDR1 of SEQ ID NO:61;—a CDR2 of SEQ ID NO:62;—a CDR3 of SEQ ID NO:63; and wherein said hypervariable region of said T cell receptor beta chain comprises:—a CDR1 of SEQ ID NO:66;—a CDR2 of SEQ ID NO:67;—a CDR3 of SEQ ID NO:68.

In another preferred embodiment of said engineered T cell of the invention, said T cell receptor alpha chain comprises an amino acid sequence of SEQ ID NO:69 and wherein said T cell receptor beta chain comprises an amino acid sequence of SEQ ID NO:70; preferably wherein said T cell receptor alpha chain comprises an amino acid sequence of SEQ ID NO:60 (optionally without the leader sequence as shown in FIG. 14 or with an alternative leader sequence) and wherein said T cell receptor beta chain comprises an amino acid sequence of SEQ ID NO:65 (optionally without the leader sequence as shown in FIG. 14 or with an alternative leader sequence).

In another embodiment of a T cell of the invention, said T cell receptor alpha chain comprises an amino acid sequence of SEQ ID NO:21 and/or said T cell receptor beta chain comprises an amino acid sequence of SEQ ID NO:22; preferably wherein said T cell receptor alpha chain is that of SEQ ID NO:11 (optionally without the leader sequence as shown in FIG. 6 or with an alternative leader sequence) and/or wherein said T cell receptor beta chain is that of SEQ ID NO:16 (optionally without the leader sequence as shown in FIG. 6 or with an alternative leader sequence).

In another preferred embodiment of said engineered T cell of the invention, said T cell epitope forms a complex with a human Major Histocompatibility Complex (MHC) molecule, preferably an HLA-A*02 molecule.

In a preferred embodiment of an engineered T cell of the invention, said T cell is additionally engineered to express (i) a further (e.g., non-TCR or non-CAR) transgene that encodes an intracellular, membrane-expressed or secreted (e.g. secretory) protein (see e.g. Kunert et al., Oncoimmunol, 7(1):e1378842 (2017)). As an example, said engineered T cell can be additionally engineered to express a further TCR or further antibody-based receptor that binds to a different T cell epitope (i.e. is dual-targeting), or said engineered T cell can be additionally engineered to express a secretable protein that is not a TCR or antibody.

In another aspect, the invention provides a TCR protein or antibody-based receptor protein, wherein said TCR protein or antibody-based receptor protein comprises a TCR or antibody-based receptor as defined in any one of the aspects and/or embodiments of an engineered T cell of the invention; preferably wherein said TCR has a T cell receptor alpha chain and a T cell receptor beta chain as disclosed herein; preferably wheren said TCR protein or antibody-based receptor protein is part of an antibody drug conjugate (ADC) or is (part of) a soluble TCR. In another aspect, the invention provides a T cell receptor (TCR) protein, wherein said TCR protein has a T cell receptor alpha chain and a T cell receptor beta chain as defined in any one of the aspects and/or embodiments of an engineered T cell of the invention.

In embodiments, the TCR protein or antibody-based receptor protein is membrane-expressed, soluble or part of a larger soluble compound, such as an antibody-drug conjugate, e.g. a TCR-like antibody-drug conjugate. The invention also provides an antibody-drug conjugate, e.g. a TCR-like antibody drug conjugate, that comprises a TCR protein or antibody-based receptor protein of the invention.

In another aspect, the invention provides a T cell receptor (TCR) alpha chain or beta chain protein, wherein said TCR alpha chain protein or beta chain protein is as defined in any one of the aspects and/or embodiments of an engineered T cell of the invention.

In another aspect, the invention provides a T cell receptor (TCR) protein or an antibody-based receptor, e.g. chimeric antigen receptor (CAR), protein that binds to a T cell epitope of human ropporin-1A (ROPN1) and/or human ropporin-1B (ROPN1B); wherein said T cell epitope consists of the amino acid sequence selected from one of SEQ ID NO:4, SEQ ID NO:43, SEQ ID NO:23, SEQ ID NO:56 and SEQ ID NO:24.

In another aspect, the invention provides a nucleic acid molecule comprising a nucleic acid sequence that encodes (i) a T cell receptor alpha chain and/or a T cell receptor beta chain or (ii) a TCR protein or antibody-based receptor as defined in any one of the aspects and/or embodiments of an engineered T cell of the invention.

In another aspect, the invention provides a nucleic acid molecule comprising a nucleic acid sequence that encodes a TCR or antibody-based receptor, e.g. CAR, protein that is specific for, or binds to, a T cell epitope that consists of the amino acid sequence selected from one of SEQ ID NO:4, SEQ ID NO:43, SEQ ID NO:23, SEQ ID NO:56 and SEQ ID NO:24.

In another aspect, the invention provides a TCR transgene that is modified (e.g. via the addition, deletion and/or substitution of one or more amino acid residues in the transmembrane and/or intracellular domains) without affecting the amino acid sequence of the TCR variable alpha and beta chains as disclosed herein (e.g. Govers et al., J Immunol, 193(10):5315-26 (2014)).

In another aspect, the invention provides a nucleic acid molecule comprising a nucleic acid sequence that encodes a TCR or antibody-based receptor, e.g. CAR, protein specific for any epitope as disclosed herein, preferably epitopes 4, 10 or 11.

In a preferred embodiment of said nucleic acid molecule of the invention, said nucleic acid molecule is part of an expression vector, such as a plasmid.

In another preferred embodiment of said nucleic acid molecule of the invention, said nucleic acid molecule is part of a retroviral plasmid expression vector, such as a pMP71 vector.

In embodiments, said nucleic acid molecule as disclosed herein is transfected into a T cell, such as a T cell obtained from the subject to be treated.

In a preferred embodiment of a T cell of the invention, said T cell is genetically engineered to express a construct encoding said TCR or said antibody-based receptor that binds to said T cell epitope of human ROPN1B and/or ROPN1.

In another preferred embodiment of a T cell of the invention, said T cell is genetically engineered to express a nucleotide sequence (preferably in the form of a nucleotide construct, such as a DNA construct, that is optionally comprised in an expression vector, for instance an expression vector that allows for integration of said nucleotide sequence into host chromosome DNA or an expression vector that remains extrachromosomal) encoding said TCR or said antibody-based receptor (such as a CAR) that binds to said T cell epitope of human ROPN1B and/or ROPN1.

In another preferred embodiment of a T cell of the invention, said T cell is genetically engineered to express said TCR that binds to a T cell epitope of human ROPN1B and/or ROPN1, wherein said TCR is with or without modifications (e.g. wherein the modification is an addition, deletion and/or substitution of one or more amino acid residues) to enhance surface expression and/or epitope-specific functions of said TCR.

In another preferred embodiment of a T cell of the invention, said T cell epitope is a peptide which forms a complex with a human Major Histocompatibility Complex (MHC) molecule, preferably an HLA-A*02 molecule.

In another preferred embodiment of a T cell of the invention, said T cell epitope consists of the amino acid sequence selected from one of SEQ ID NOs:1-9, 20, 23-32, 43 or 56 (preferably one of SEQ ID NOs:4, 23, 24, 43 or 56) and sequences having at least 70%, or at least 80%, sequence identity thereto. In the epitope motifs of SEQ ID NOs: 20,43 and 56, “X” can be any amino acid residue, such as alanine.

In an embodiment of a T cell of the invention, said T cell epitope consists of the amino acid sequence selected from SEQ ID NO:1 and modified amino acid sequences thereof in which the amino acid residue at position 6 (Glu), position 8 (Glu) and/or position 9 (Val) of SEQ ID NO:1 is substituted by another amino acid residue. In a more preferred embodiment, said T cell epitope consists of the amino acid sequence selected from SEQ ID NO:4 and modified amino acid sequences thereof in which the amino acid residue at position 1 (F), position 2 (L) and/or position 9 (V) of SEQ ID NO:4 is substituted by another amino acid residue. In another preferred embodiment, said T cell epitope consists of the amino acid sequence selected from SEQ ID NO:23 and modified amino acid sequences thereof in which the amino acid residue at position 1 (F) and/or position 9 (V) of SEQ ID NO:23 is substituted by another amino acid residue.

In another preferred embodiment of a T cell of the invention, said T cell is a CD8+ T cell. Preferably, said T cell is gene-engineered to express said TCR. Preferably, a T cell of the invention expresses said TCR, preferably on the surface of said T cell. Said TCR is thereby able to react specifically to a ROPN1 and/or ROPN1B epitope as disclosed herein.

In another preferred embodiment of a T cell of the invention, said T cell is a human T cell.

In another aspect, said T cell as disclosed herein is for use in autologous T cell therapy.

In another aspect, the invention provides a collection of T cells, comprising a multitude of T cells as disclosed herein.

In another aspect, the invention provides a pharmaceutical composition comprising an engineered T cell as disclosed herein, and a pharmaceutically acceptable excipient such as a carrier or diluent.

In another aspect, the invention provides an engineered T cell of the invention, a pharmaceutical composition of the invention, a TCR protein of the invention or a nucleic acid molecule of the invention, for use in therapy or for use as a medicament.

In another aspect, the invention provides a (gene-engineered) T cell as disclosed herein for use in therapy such as autologous T cell therapy, preferably for use in the treatment of a solid tumor or liquid tumor.

In a preferred embodiment, the engineered T cell, pharmaceutical composition, TCR protein or nucleic acid molecule of the invention are for use in the treatment of a tumor, preferably a solid tumor or a liquid tumor. More preferably, the tumor is malignant, i.e., a cancer.

In a preferred embodiment of a T cell, pharmaceutical composition, TCR protein or nucleic acid molecule for use of the invention, said tumor comprises tumor cells expressing human ROPN1 and/or ROPN1B, preferably wherein said tumor comprises tumor cells that comprise an MHC molecule that is in complex with, or bound to, a T cell epitope as disclosed herein, preferably a T cell epitope selected from the group consisting of SEQ ID NOs:4, 23, 24, 43 or 56, more preferably wherein said T cell epitope is SEQ ID NO:4.

In another preferred embodiment of a T cell, pharmaceutical composition, TCR protein or nucleic acid molecule for use of the invention, said solid tumor is a breast cancer, preferably a triple negative breast cancer (TNBC), or a skin cancer, preferably a melanoma such as a skin cutaneous melanoma (SKCM).

In another preferred embodiment of a T cell, pharmaceutical composition, TCR protein or nucleic acid molecule for use of the invention, said liquid tumor is a myeloma, preferably a multiple myeloma, a leukemia, preferably an acute myeloid leukemia, or a lymphoma.

In another aspect, the invention provides a T cell receptor (TCR) protein or antibody-based receptor protein (such as a CAR protein) as defined in any one of the previous or subsequent aspects and/or embodiments relating to a T cell of the invention. In the same manner, the invention provides a TCR alpha chain and/or a TCR beta chain protein of a T cell receptor (TCR) protein of the invention.

In a preferred embodiment of a TCR protein or antibody-based receptor protein of the invention, said TCR protein or antibody-based receptor protein binds to a T cell epitope of human ropporin-1B (ROPN1B) and/or human ropporin-1A (ROPN1), preferably wherein said T cell epitope consists of the amino acid sequence of any one of SEQ ID NOs:1-9, 20, 23-32, 43 or 56 (preferably one of SEQ ID NOs:4, 23, 24, 43 or 56) or sequences having at least 70% or at least 80% sequence identity thereto.

In another preferred embodiment of a TCR protein or antibody-based receptor protein of the invention, said TCR protein or said antibody-based receptor protein binds to a T cell epitope that consists of the amino acid sequence selected from (i) SEQ ID NO:1 and modified amino acid sequences thereof in which the amino acid residue at position 6 (Glu), position 8 (Glu) and/or position 9 (Val) of SEQ ID NO:1 is substituted by another amino acid residue, (ii) SEQ ID NO:4 or modified amino acid sequences thereof in which the amino acid residue at position 1 (F), position 2 (L) and/or position 9 (V) of SEQ ID NO:4 is substituted by another amino acid residue, (iii) SEQ ID NO:23 or modified amino acid sequences thereof in which the amino acid residue at position 1 (F) and/or position 9 (V) of SEQ ID NO:23 is substituted by another amino acid residue, or (iv) SEQ ID NO:24.

In an embodiment of a TCR protein of the invention, said TCR protein comprises: (i) a T cell receptor alpha chain comprising a hypervariable region that comprises a CDR3 of SEQ ID NO:14, and (ii) a T cell receptor beta chain comprising a hypervariable region that comprises a CDR3 of SEQ ID NO:19, wherein said hypervariable regions of said T cell receptor alpha and beta chain further comprise a CDR1 and a CDR2.

In another embodiment of a TCR protein of the invention, said hypervariable region of said TCR alpha chain comprises:—a CDR1 of SEQ ID NO:12;—a CDR2 of SEQ ID NO:13;—a CDR3 of SEQ ID NO:14; and/or wherein said hypervariable region of said T cell receptor beta chain comprises:—a CDR1 of SEQ ID NO:17;—a CDR2 of SEQ ID NO:18; and—a CDR3 of SEQ ID NO:19.

In another embodiment of a TCR protein of the invention, said T cell receptor alpha chain comprises an amino acid sequence of SEQ ID NO:21 and/or said T cell receptor beta chain comprises an amino acid sequence of SEQ ID NO:22; preferably wherein said T cell receptor alpha chain is that of SEQ ID NO:11 and/or wherein said T cell receptor beta chain is that of SEQ ID NO:16.

In a preferred embodiment of a TCR protein of the invention, said TCR comprises: (i) a T cell receptor alpha chain comprising a hypervariable region that comprises a CDR3 of SEQ ID NO:37, and (ii) a T cell receptor beta chain comprising a hypervariable region that comprises a CDR3 of SEQ ID NO:42; and wherein said hypervariable regions of said T cell receptor alpha and beta chain further comprise a CDR1 and a CDR2.

In another preferred embodiment of a TCR protein of the invention, said hypervariable region of said TCR alpha chain comprises:—a CDR1 of SEQ ID NO:35;—a CDR2 of SEQ ID NO:36;—a CDR3 of SEQ ID NO:37; and wherein said hypervariable region of said T cell receptor beta chain comprises:—a CDR1 of SEQ ID NO:40;—a CDR2 of SEQ ID NO:41;—a CDR3 of SEQ ID NO:42.

In another preferred embodiment of a TCR protein of the invention, said T cell receptor alpha chain comprises an amino acid sequence of SEQ ID NO:44 and wherein said T cell receptor beta chain comprises an amino acid sequence of SEQ ID NO:45; preferably wherein said T cell receptor alpha chain comprises an amino acid sequence of SEQ ID NO:34 and wherein said T cell receptor beta chain comprises an amino acid sequence of SEQ ID NO:39.

In another preferred embodiment of a TCR protein of the invention, said TCR comprises: (i) a T cell receptor alpha chain comprising a hypervariable region that comprises a CDR3 of SEQ ID NO:50, and (ii) a T cell receptor beta chain comprising a hypervariable region that comprises a CDR3 of SEQ ID NO:55; and wherein said hypervariable regions of said T cell receptor alpha and beta chain further comprise a CDR1 and a CDR2.

In another preferred embodiment of a TCR protein of the invention, said hypervariable region of said TCR alpha chain comprises: a CDR1 of SEQ ID NO:48;—a CDR2 of SEQ ID NO:49;—a CDR3 of SEQ ID NO:50; and wherein said hypervariable region of said T cell receptor beta chain comprises:—a CDR1 of SEQ ID NO:53;—a CDR2 of SEQ ID NO:54;—a CDR3 of SEQ ID NO:55.

In another preferred embodiment of a TCR protein of the invention, said T cell receptor alpha chain comprises an amino acid sequence of SEQ ID NO:57 and wherein said T cell receptor beta chain comprises an amino acid sequence of SEQ ID NO:58; preferably wherein said T cell receptor alpha chain comprises an amino acid sequence of SEQ ID NO:47 and wherein said T cell receptor beta chain comprises an amino acid sequence of SEQ ID NO:52.

In another preferred embodiment of a TCR protein of the invention, said TCR comprises: (i) a T cell receptor alpha chain comprising a hypervariable region that comprises a CDR3 of SEQ ID NO:63, and (ii) a T cell receptor beta chain comprising a hypervariable region that comprises a CDR3 of SEQ ID NO:68; and wherein said hypervariable regions of said T cell receptor alpha and beta chain further comprise a CDR1 and a CDR2.

In another preferred embodiment of a TCR protein of the invention, said hypervariable region of said TCR alpha chain comprises:—a CDR1 of SEQ ID NO:61;—a CDR2 of SEQ ID NO:62;—a CDR3 of SEQ ID NO:63; and wherein said hypervariable region of said T cell receptor beta chain comprises:—a CDR1 of SEQ ID NO:66;—a CDR2 of SEQ ID NO:67;—a CDR3 of SEQ ID NO:68.

In another preferred embodiment of a TCR protein of the invention, said T cell receptor alpha chain comprises an amino acid sequence of SEQ ID NO:69 and wherein said T cell receptor beta chain comprises an amino acid sequence of SEQ ID NO:70; preferably wherein said T cell receptor alpha chain comprises an amino acid sequence of SEQ ID NO:60 and wherein said T cell receptor beta chain comprises an amino acid sequence of SEQ ID NO:65.

In a preferred embodiment of an antibody-based receptor, e.g. CAR, protein of the invention, said antibody-based receptor protein binds to an epitope of SEQ ID NO:4; preferably wherein said antibody-based receptor protein comprises one or more CDRs, preferably all, selected from the group consisting of SEQ ID NOs:35, 36, 37, 40, 41 and 42. In other embodiments of said antibody-based receptor protein of the invention, at least SEQ ID NO:37 and/or SEQ ID NO:42 are present.

In a preferred embodiment of an antibody-based receptor, e.g. CAR, protein of the invention, said antibody-based receptor protein binds to an epitope of SEQ ID NO:23, preferably wherein said antibody-based receptor protein comprises one or more CDRs, preferably all, selected from the group consisting of SEQ ID NOs:48, 49, 50, 53, 54 and 55. In other embodiments of said antibody-based receptor protein of the invention, at least SEQ ID NO:50 and/or SEQ ID NO:55 are present.

In a preferred embodiment of an antibody-based receptor, e.g. CAR, protein of the invention, said antibody-based receptor protein binds to an epitope of SEQ ID NO:24, preferably wherein said antibody-based receptor protein comprises one or more CDRs, preferably all, selected from the group consisting of SEQ ID NOs:61, 62, 63, 66, 67 and 68. In other embodiments of said antibody-based receptor protein of the invention, at least SEQ ID NO:63 and/or SEQ ID NO:68 are present.

In a preferred embodiment of a TCR protein or antibody-based receptor protein of the invention, said TCR protein or antibody-based receptor protein is an isolated or purified TCR protein or antibody-based receptor protein.

In an embodiment of a T cell or TCR protein of the invention, said TCR alpha and beta chains are encoded by one or more nucleotide sequences, such as, but not limited to, those provided in SEQ ID NO:10 and 15, respectively, and the translated protein comprises a TCR alpha chain variable sequence of SEQ ID NO:11 and a TCR beta chain variable sequence of SEQ ID NO:16. In a preferred embodiment of an engineered T cell or TCR protein of the invention, said TCR alpha and beta chains are encoded by one or more nucleotide sequences, such as, but not limited to, those provided in SEQ ID NOs: 33 and 38, respectively, and the translated protein comprises a TCR alpha chain variable sequence of SEQ ID NO:44 and a TCR beta chain variable sequence of SEQ ID NO:45. In another preferred embodiment of a T cell or TCR protein of the invention, said TCR alpha and beta chains are encoded by one or more nucleotide sequences, such as, but not limited to, those provided in SEQ ID NOs: 46 and 51, respectively, and the translated protein comprises a TCR alpha chain variable sequence of SEQ ID NO:57 and a TCR beta chain variable sequence of SEQ ID NO:58. In another preferred embodiment of a T cell or TCR protein of the invention, said TCR alpha and beta chains are encoded by one or more nucleotide sequences, such as, but not limited to, those provided in SEQ ID NOs: 59 and 64, respectively, and the translated protein comprises a TCR alpha chain variable sequence of SEQ ID NO:69 and a TCR beta chain variable sequence of SEQ ID NO:70.

The invention also provides an isolated or purified peptide (T cell epitope) of human ropporin-1B (ROPN1B) and/or human ropporin-1A (ROPN1), which forms a complex with a human Major Histocompatibility Complex (MHC) molecule, preferably an HLA-A*02 molecule.

In a preferred embodiment of said isolated or purified peptide of the invention, said peptide consists of the amino acid sequence of any one of SEQ ID NO:4, SEQ ID NO:43, SEQ ID NO:23, SEQ ID NO:56 and SEQ ID NO:24.

In an embodiment of a peptide of the invention, said peptide consists of the amino acid sequence of any one of SEQ ID NOs:1-9, 20, 23-32, 43 or 56 (preferably one of SEQ ID NOs:4, 23, 24, 43 or 56) or sequences having at least 70% or at least 80% sequence identity thereto, or a modified amino acid sequence of SEQ ID NOs:1, 4, 23 and 24 as defined above.

The invention also provides an isolated or synthesized human MHC molecule in complex with a peptide (T cell epitope) of human ropporin-1B (ROPN1B) and/or human ropporin-1A (ROPN1) of the invention.

The invention also provides an immunogenic composition comprising a peptide of human ropporin-1B (ROPN1B) and/or human ropporin-1A (ROPN1) of the invention and/or an MHC molecule of the invention, said composition further comprising a pharmaceutically acceptable excipient; said composition optionally further comprising an adjuvant; preferably wherein said composition is for use in vaccination, more preferably for use in vaccination of a subject against a tumor as disclosed herein such as breast cancer or skin cancer.

The invention also provides an engineered cell, preferably an engineered cancer cell, wherein said cell is engineered to express human ropporin-1B (ROPN1B) and/or human ropporin-1A (ROPN1).

The invention also provides a nucleic acid encoding a TCR alpha and/or a TCR beta chain of a TCR protein or antibody-based receptor protein of the invention with or without modifications to enhance surface expression and/or epitope-specific functions, or a nucleic acid encoding a peptide of human ropporin-1B (ROPN1B) and/or human ropporin-1A (ROPN1) of the invention.

Also provided is a method of treating a subject suffering, or suspected of suffering, from a tumor, comprising the step of:—administering a therapeutically effective amount of a T cell according to the invention or a pharmaceutical composition of the invention to a subject in need thereof.

The invention also provides a method for binding a T cell as disclosed herein to a T cell epitope as disclosed herein in a subject suffering, or suspected of suffering, from a solid tumor, comprising the step of: administering a T cell as disclosed herein to said subject. In these embodiments, the solid tumors in said subjects express the epitope on their surface, e.g. as a surface antigen. The present invention also provides a method of producing an epitope-specific T-cell, wherein said epitope is a human ropporin-1B (ROPN1B) and/or human ropporin-1A (ROPN1) epitope as disclosed herein, the method comprising the steps of generating epitope-specific T cells by bringing an epitope expressing cell, which cell presents the ROPN1B and/or ROPN1 epitope on its cell surface, optionally HLA presented, in contact with a population of T cells, wherein said population of T cells is either a population of autologous host T cells or allogeneic host T cells, selecting the T cells, preferably CD8+ T cells that bind to the cell that presents the ROPN1B and/or ROPN1 epitope, and optionally enriching and/or propagating the selected T cells thus provided. In addition, the method may comprise the steps of sequencing the TCR-encoding gene(s), and cloning said gene as a transgene in a recipient T cell to provide a genetically engineered T cell expressing the ROPN1B and/or ROPN1 epitope-specific TCR.

The invention also provides a use of a T cell of the invention, pharmaceutical composition of the invention, TCR protein of the invention, antibody-based receptor protein of the invention or a nucleic acid molecule of the invention in the manufacture of a medicament for the treatment of a tumor (e.g. solid tumor or liquid tumor) in a subject.

The invention also provides an engineered T cell, expressing a T cell receptor (TCR) that binds to a T cell epitope of human ropporin-1B (ROPN1B), wherein said TCR comprises: (i) a T cell receptor alpha chain comprising a hypervariable region that comprises a CDR3 of SEQ ID NO:14, and (ii) a T cell receptor beta chain comprising a hypervariable region that comprises a CDR3 of SEQ ID NO:19, wherein said hypervariable regions of said T cell receptor alpha and beta chain further comprise a CDR1 and a CDR2.

The invention also provides an engineered T cell, expressing a T cell receptor (TCR) or antibody-based receptor (such as a CAR) that binds to a T cell epitope of human ropporin-1B (ROPN1B) and/or human ropporin-1A (ROPN1).

In another aspect, the invention provides an (optionally isolated or purified) immune cell such as a T cell, wherein said T cell expresses a T cell receptor (TCR) that binds to a T cell epitope of human ropporin-1A (ROPN1) and/or human ropporin-1B (ROPN1B); wherein said T cell epitope consists of the amino acid sequence selected from one of SEQ ID NO:4, SEQ ID NO:43, SEQ ID NO:23, SEQ ID NO:56 and SEQ ID NO:24. In an embodiment of said aspect, the immune cell, preferably T cell, is not engineered to express said T cell receptor (TCR), but e.g. natively expresses said TCR, e.g. said TCR protein as disclosed herein.

In other embodiments, the immune cell, preferably T cell, may be additionally, e.g. besides said TCR transgene, engineered to express a different (i.e. non-TCR) transgene, and/or said TCR transgene may be modified (e.g. via the addition, deletion and/or substitution of one or more amino acid residues without affecting the said TCR alpha and beta variable domains) to enable enhanced anti-tumor responses of said T cell (vide Govers et al., J Immunol., 193(10):5315-26 (2014)).

In another aspect, the invention provides a pharmaceutical composition comprising said immune cell.

### DESCRIPTION OF THE DRAWINGS

FIG. 1. ROPN1 and ROPN1B are not expressed in healthy tissues. A. Bars show ROPN1 and ROPN1B gene expression in healthy tissues according to TPM values and based on RNAseq of 6 different healthy tissue databases (filled boxes indicate presence of a tissue in a database, see Example 1, Materials and Methods for details), dashed, orange line shows expression of NY-ESO1 which was used as a threshold value. B. Dots (superimposed) show gene expression of ROPN1 and ROPN1B expressed as fold change in comparison to GAPDH in 48 healthy tissues according to qPCR using a cDNA library of healthy tissue samples. C. Panels show representative immune stainings of healthy tissues using ROPN1 antibody (which detects both ROPN1 and ROPN1B) on tissue microarrays (TMAs, n=63). In gene and protein expression analyses, NY-ES01 was taken along as a control.

FIG. 2. ROPN1 and ROPN1B show high and homogenous expression in TNBC. A. Bar graphs show fraction of TNBC tumors with weak (TPM 1-10), moderate (TPM 10-100) and strong (TPM>100) gene expression of ROPN1 and ROPN1B (TCGA, RNAseq, n=122, see Example 1, Materials and Methods for details). B. Bar graphs show fraction of TNBC tumors with weak, moderate and strong immune staining of ROPN1 and ROPN1B (TMAs, n=338); scoring is performed as described in Example 1, Materials and Methods. C. Bar graphs show fraction of TNBC tumors with either 1-9%, 10-25%, 26-50% or 51-100% of tumors cells positive for ROPN1 and ROPN1B protein. D. Panels are representative of TNBC tumors with weak, moderate and strong immune stainings of ROPN1 and ROPN1B. E+F. Bar graphs show fraction of tumors with weak, moderate and strong gene expression of ROPN1B in 14 tumor types (as in panel A, TCGA, RNAseq data, n=6670). In gene and protein expression analyses, NY-ESO1 was taken along as a control. Abbreviations: BLCA, Bladder Urothelial Carcinoma; BRCA, Breast Carcinoma; COAD, Colon Adenocarcinoma; GBM, Glioblastoma Multiforme; KIRC, Kidney Renal Clear Cell Carcinoma; LIHC, Liver Hepatocellular Carcinoma; LUAD, Lung Adenocarcinoma; LUSC, Lung Squamous Cell Carcinoma; OV, Ovarian Serous Cystadenocarcinoma; PAAD, Pancreatic Adenocarcinoma; PRAD, Prostate Adenocarcinoma; SKCM, Skin Cutaneous Melanoma; THCA, Thyroid Carcinoma; UCEC, Uterine Corpus Endometrial Carcinoma.

FIG. 3. Prediction and selection of ROPN1 and ROPN1B epitopes that are immunogenic, safe and bind to HLA-A2. A. Flow chart of ROPN1 and ROPN1B peptide selection based on in silico predictions, peptide elution, and check for non-cross-reactivity and HLA-A2 bindings (see Example 1, Materials and Methods for details on each tool/assay, and Table 1 for details on non-cross-reactivity of epitopes). B. ROPN1B staining of MDA-MB231 TNBC cell line with and without ROPN1B overexpression either grown on spins (left, cytospin staining) or in suspension (right, flowcytometry, red indicates % of GFP-positive cells of the overexpressing cell line, blue indicates the negative control). Histogram shows the total number of peptides (y-axis) and their length (x-axis) eluted from MDA-MB231-ROPN1B+GFP cells (below). C. Head-to-head comparisons of unique (non-cross reactive) epitopes for HLA-A2 binding stability. Test was performed using single peptide concentrations (25 PI); results are expressed as fold change of median fluorescent intensity (MFI) of anti-HLA-A2-PE over baseline (T2 cells without added peptide), n=6. Peptides with fold change >1.1 over no peptide were tested using titrated amounts (range from: 31 nM to 31 μM). Gp100 peptide YLE was used as a positive control and NY-ESO peptide SLL as a reference peptide. Representative titration curves are shown in panel D. E. Tabular overview of epitope rankings. Input comprised control/reference peptides; 14 ROPN1 and ROPN1B peptides that were obtained following predictions for immunogenicity or elutions, and cross-reactivity testing (see panel A). The table includes from left-to-right: in silico scores of each tool (provided as ranks); HLA-A2 binding parameters, such as minimum stability, amplitude (highest data point corrected for baseline point), and EC50 values (in M, calculated with GraphPad software 5); and a final ranking of epitopes (thresholds being: half of the amplitude of reference peptide YLE; and EC50 value below 1E-05). Peptides not reaching thresholds were ranked based on their EC50 values.

FIG. 4. Enrichment of ROPN1 and ROPN1B epitope-specific CD8+ T cells and their TCRs. A. Flow chart shows individual steps from T cell enrichment towards the identification of TCR genes. B. Boxplots show IFNy production of epitope 1 (SEQ ID NO:1)-stimulated T cells that were enriched following 4 or 5 cycles of co-culture with epitope 1-loaded aAPCs (see Example 1, Materials and Methods for details). C. Representative peptide:MHC-staining of T cells following 5 cycles of of co-culture with epitope 1-loaded aAPCs. pMHC+CD8 T cells were gated based on fluorescence minus one (FMO, which contained all markers except for pMHC). D. Flow cytometry plots show staining of T cells from panel B with pMHC (MLN/A2 complexes) (control) as well as T cells derived from different clones following IFNy capture (clones 1-8, see Example 1, Materials and Methods for details). Samples with yellow squares (clone 2 and clone 8) were used for 5′RACE PCR and TCR sequencing. E. Flow cytometry plots show staining of T cells from panel B with pMHC following FACS-sort with MLN/A2-pMHC multimers as well as the corresponding fluorescence minus one (FMO) control. Sample with yellow square was used for 5′RACE PCR and sequencing. F. Gel shows bands of 5′RACE products for TCR alpha and beta genes. +indicates positive PCR control; a and 13 indicate RACE products for TCR alpha and beta genes for clones 2 and 8 from limiting dilution and the pMHC FACS-sorted (F) population. The right gel shows an additional amplification step using nested primers. G. Identified T cell receptor V-alpha (TRAV and J according to IMGT nomenclature; yellow) and beta genes (TRBV, D and J; blue) as well as corresponding C genes (starting and ending amino acids) cloned from T cells from panels E and F; percentage reflects fraction of all identified colonies.

FIG. 5: Strict epitope specificity of T cells gene-engineered to express ROPN1(B) MLN TCRs. A. Gene transfer of MLN-TCR1 and MLN-TCR2 (MLN-TCR2 is a TCR with alpha chain of SEQ ID NO:11 and beta chain of SEQ ID NO:16) in T cells from 2 healthy donors, and binding of MLN/A2-pMHC multimer as determined by flow cytometry. B. MACS-sort of MLN-TCR2 T cells from 2 healthy donors; panels show MLN/A2-pMHC binding before (left) and after MACS-sort with pMHC (right). C. Representative IFN_{Y} response of MLN-TCR2 T cells (one out of 2 donors) upon stimulation with titrated amounts of epitope 1 (MLN) and gp100 peptides (n=3). D. Bargraphs show IFNy-production by MLN-TCR2 T cells upon recognition of epitope 1 (MLN) from ROPN1B yet no IFNg production when epitope was derived from ROPN1 (representative graph from n=3). E. Bargraphs show IFNy production by MLN-TCR2 T cells in response to cognate peptide with alanine-mutations at each individual position relative to the unmutated cognate peptide (representative graph from n=3).

FIG. 6: SEQ ID NOs:10, 11, 15 and 16 annotated for distinct regions. Leader sequence, TRAV, TRAJ and TRAC domains are shown for TCR alpha chain of SEQ ID NO:10 (nucleotide sequence) and 11 (amino acid sequence). Leader sequence, TRBV, TRBD, TRBJ and TRBC domains are shown for TCR beta chain of SEQ ID NO:15 (nucleotide sequene) and 16 (amino acid sequence). CDR 1-3 regions are shown in bold.

FIG. 7 (Extension to FIG. 3). Selection of predicted and eluted ROPN1 and ROPN1B epitopes according to immunogenicity, safety and binding to HLA-A2. A. Flow chart of ROPN1 and ROPN1B peptide selection based on in silico predictions, peptide elution (total n=28), as well as checks for non-cross-reactivity (n=19), minimum binding characteristics for HLA-A2 (n=11), and ranking according to amplitude of HLA-A2 binding (see Example 1, Materials and Methods for details on each tool/assay, and Table 3 for details on non-cross-reactivity of epitopes). B. Head-to-head comparisons of unique (non-cross reactive) epitopes for HLA-A2 binding. Tests were performed using single epitope concentrations (31 PI); results are expressed as fold change of median fluorescent intensity (MFI) of bound anti-HLA-A2-PE over baseline (T2 cells without added epitope), n=2/3 per epitope. C. Epitopes with fold change >1.1 relative to no epitope were further tested with T2 cells using titrated amounts of epitope (range from: 31 nM to 31 μM). Representative titration curves are shown. Gp100 peptide (YLE) was used as a reference epitope. D. Overview of epitopes and their (from left-to-right): in silico scores (provided as ranks); HLA-A2 binding scores (i.e., minimum stability (see above), amplitude (relative to amplitude of reference epitope), and EC50 values (in Molarity, calculated with GraphPad software 5)); and a final ranking of epitopes. When epitopes adhered to the following 3 criteria, and demonstrated (1) HLA-A2 binding stability of >1.1 relative to no peptide; (2) EC50 of <5×10⁻⁵ M; and (3) binding amplitude of >0.5 relative to reference peptide YLE (see panels B and C), then remaining epitopes (n=11) were ranked according to amplitude values.

FIG. 8. Flow chart with individual steps from enrichment of ROPN1 and ROPN1B-specific CD8+ T cells to testing of sensitivity and specificity of corresponding TCRs. Cartoon illustrating in 8 steps how ROPN1N and ROP1NB-specific CD8+ T cells are retrieved, the corresponding TCRs identified and tested according to in vitro and in vivo assays for sensitivity and specificity. Per step, the inclusion criteria are displayed that need to be reached for epitope-specific T cells or TCRs to move to the next step. Those T cells or TCRs directed against epitopes that reach each step are highlighted in bold.

FIG. 9 (Extension to FIGS. 4 and 5). Enrichment of ROPN1 and ROPN1B epitope-specific CD8+ T cells and identification and gene transfer of corresponding TCRs. ROPN1 and ROPN1B epitopes that were ranked in FIG. 3D were used to start enrichments for epitope-specific CD8+ T cells. The epitopes with SEQ ID NOs:1 to 9, 23 and 24 are positioned vertically, and results are positioned horizontally. Results per epitope (from left to right) are: (i) epitope-specific IFNg production and (ii) peptide:MHC binding of CD8+ T cells; sequences of clonal TCRs of FACSorted CD8+ T cells; and (iv) surface expression of TCRs following gene transfer into T cells. IFNg levels (in pg/ml) were determined with ELISA at 24 h following T cell stimulation with T2 cells loaded with cognate or random epitope. Binding of pMHC by CD8+ T cells (in %) was determined following staining with peptide:MHC tetramer, and flow cytometric analysis. TCR-V-alpha (TRAV and J according to IMGT nomenclature; yellow) and beta genes (TRBV, D and J; blue) were sequenced following 5′RACE PCR of cDNA from FACSorted pMHC+ T cells; percentage reflects fraction of all identified colonies. TCR expression is determined in healthy donor T cells that were retrovirally transduced with TCR genes, after which T cells were stained with peptide:MHC. Representative flow plots are shown (1 out of 2 donors). In case of epitope 11 (SEQ ID NO:24): the specific peptide:MHC complexes appeared insensitive in detecting TCR T cells, and were replaced by stainings with antibodies directed against TCR-Vb7.1 and CD137 (the latter following 48 h stimulation with cognate epitope-loaded BSM cells). Shown is the anti-epitope 11 TCRab that showed CD137 response. For details see: FIG. 8, Table 4 and Example 1, Materials and Methods. NA means not applicable, i.e., T cells or TCRs did not reach the inclusion criteria of the pervious step.

FIG. 10 (Extension to FIG. 5). Sensitivity towards cognate epitope of T cells gene-engineered to express ROPN1 and B-restricted TCRs. ROPN1 and ROPN1B epitopes that showed TCR surface expression in FIG. 5 were used to test sensitivity towards the cognate epitope. The epitopes with SEQ ID NOs 1, 4, 8, 23 and 24 are positioned vertically, and results are positioned horizontally. Results per epitope (from left to right) are IFNg production upon stimulation with: (i) ROPN1 or ROPN1B-transfected breast cancer cell line; and (ii) BSM cells loaded with titrated amounts of cognate epitope. IFNg levels were determined with ELISA (in pg/ml). Controls for (i) include BSM cells loaded with cognate or random epitope. TCR T cell reactivity against the TNBC cell line MM231 transfected with ROPN1 or ROPN1B provides a measure that the epitope is recognized following endogenous antigen processing and presentation (in other words, the epitope does not represent an artificial epitope). In (ii) BSM cells were loaded with cognate epitope ranging from 1 nM to 30 μM. EC50 values are expressed in Molarity, and calculated with GraphPad software 5, and represent a measure of sensitivity for TCR T cells towards the cognate epitope. Gp100 peptide (YLE) was used as a reference peptide. For details see: FIG. 8, Table 4 and Example 1, Materials and Methods. NA means not applicable, i.e., T cells or TCRs did not reach the inclusion criteria of the pervious step.

FIG. 11 (Extension to FIG. 5). Strict epitope specificity of T cells gene-engineered to express ROPN1 and B-restricted TCRs. ROPN1 and ROPN1B epitopes that showed sensitive TCR T cell response towards the cognate epitope in FIG. 6 were used to test specificity towards the cognate epitope. The epitopes with SEQ ID NOs: 4 and 23 are positioned vertically, and results are positioned horizontally. Results per epitope (from left to right) are IFNg production upon stimulation with: (i) cognate epitope mutated at single amino acid positions; and (ii) library of HLA-A2-eluted peptides. BSM cells were loaded with 10 mM of epitopes, and IFNg levels (in pg/ml) in 24 h supernatants were measured with ELISA. In (i) TCR T cells were stimulated with cognate epitope or epitopes with a single alanine replacement (in case of alanine in original epitope, then glycine replacement). IFNg levels are displayed as mean % relative to response to non-mutated, cognate epitope±SEM (n=3). Responses<50% (dashed line) are indicative of amino acids critical for TCR recognition (recognition motif: underlined amino acids). Homologous motifs were queried against a human protein database using ScanProSite; this yielded no non-cognate matches for the TCRs tested. In (ii) TCR T cells were stimulated with 114 different HLA-A2-eluted peptides. Cognate epitopes served as positive controls. IFNg levels are displayed as mean±SEM (n=3). For details see: FIG. 8, Table 4 and Example 1, Materials and Methods. NA means not applicable, i.e., T cells or TCRs did not reach the inclusion criteria of the pervious step.

FIG. 12. Recognition of ROPN1A and B-positive 3D breast tumoroid by TCR-engineered T cells. ROPN1 and ROPN1B epitopes that showed specific TCR T cell response towards the cognate epitope in FIG. 7 were used to test reactivity towards a 3D breast tumoroid. The epitopes with SEQ ID Nos: 4 and 23 are positioned vertically, and results are positioned horizontally. Results per epitope include real-time tracking and monitoring of TCR T cells in a three-dimensional tumoroid model of breast cancer cells. Tumoroids were derived from ROPN1 or ROPN1B-transfected MM231 cells and grown in a collagen-matrix, after which TCR T cells were added directly on top of the tumoroid. Tumor cells were transfected with GFP (coupled to ROPN1 or ROPN1B; providing green color), TCR T cells were labeled with Hoechst prior addition on top of the tumoroid (providing blue color), and PI-label was used to monitor cell death (providing red color). The co-culture between TCR T cells and tumoroids were monitored at various time points by fluorescent microscopy. Representative images represent t=0, 24 and 48 h; and plots display differences in signal of GFP and PI at 48 relative to 0 h. For details see: FIG. 8, Table 4 and Example 1, Materials and Methods. NA means not applicable, i.e., T cells or TCRs did not reach the inclusion criteria of the pervious step.

FIG. 13. Regression of ROPN1-positive breast tumor following adoptive transfer of TCR-engineered T cells in immune-deficient mice. ROPN1-positive breast cancer cells (MM321) in matrigel were s.c. transplanted in the right flank of NSG mice. When tumors were palpable (˜200 mm³), mice were pretreated with an i.p. injection of busulfan (day −3) followed by cyclophosphamide (day −2). At day 0 and 3, mice received 2 transfers each of 15×10⁶ TCR or Mock-engineered human T cells i.v., followed by s.c. IL-2 injections for 8 consecutive days (n=4 per group). T cells were freshly transduced (day 0 transfer) and maintained with IL15 and IL21 (day 7 transfer). A. Waterfall plot (day 10 relative to day 0). B. Representative macroscopic example.

FIG. 14. TCR sequences specific for ROPN1 and ROPN1B epitopes 4 (SEQ ID NO:4), 10 (SEQ ID NO:23) and 11 (SEQ ID NO:24) annotated for distinct regions. Leader sequence, TRAV, TRAJ and TRAC domains are shown for TCR alpha chain of SEQ ID NO: 33, 46, 59 (nucleotide sequence) and 34, 47, 60 (amino acid sequence). Leader sequence, TRBV, TRBD, TRBJ and TRBC domains are shown for TCR beta chain of SEQ ID NO: 38, 51, 64 (nucleotide sequence) and 39, 52, 65 (amino acid sequence). CDR 1-3 regions are shown in bold.

### DETAILED DESCRIPTION OF THE INVENTION

The term “engineered”, as used herein in relation to T cells, includes references to T cells that are modified from their naturally occurring form. The modification is preferably a genetic modification, for example, wherein a T cell comprises an engineered nucleic acid sequence which provides for a protein having at least one amino acid deletion, insertion or substitution relative to naturally occurring molecules or comprises a heterologous nucleic acid sequence. Engineered T cells preferably express a TCR transgene as disclosed herein. Engineered T cells are expressly not naturally occurring T cells. The term “engineered” can be used interchangeably with “recombinant”, which means made through genetic engineering. As used herein, the term “engineered cell” or “genetically engineered cell” is used to indicate a cell that comprises at least a single nucleic acid molecule that is not found in a corresponding wild type cell or that is inserted in the genome at a position that is not found in a wild type cell. For example, an engineered cell may comprise or harbor a nucleic acid expression vector that is integrated into the genome of cells or present as an extrachromosomal genetic element.

The phrase “engineered to express a T cell receptor (TCR) or antibody-based receptor”, as used herein in relation to engineered T cells, includes the possibility that the TCR or antibody-based receptor is genetically modified (e.g. via the addition, deletion and/or substitution of one or more amino acid residues) (vide Govers et al., J Immunol, 193(10):5315-26 (2014)) or transgenic, and includes the possibility that the TCR or antibody-based receptor is or is not affinity-enhanced, and includes the possibility that the cell engineered to express a TCR or antibody-based receptor further expresses one or more additional TCRs or antibody-based receptors, for instance in the form of transgenes. The engineered T cell may further, in addition to a TCR or antibody-based receptor, express a (trans)gene that encodes an intracellular, membrane-expressed or secretable protein (e.g. Kunert et al., Oncoimmunology, 7(1):e1378842 (2017)). As an example, a further TCR (trans)gene or further antibody-based receptor (trans)gene that binds to a different epitope may be expressed.

The term “naturally occurring”, as used herein, includes reference to objects that are present in nature.

The term “T cell”, as used herein, includes reference to a thymus-derived lymphocyte that participates in a variety of cell-mediated immune reactions. The term includes reference to T helper cells (CD4+ T cells) and cytotoxic T cells (CTLs, CD8+ T cells) such as cytolytic T cells, and their various subsets. Preferably, but not exclusively, the T cell is a CD3+, CD8+ T cell. In embodiments, a T cell, or collection of T cells, prior to transfection with a TCR transgene as disclosed herein is isolated or purified, generally, but not exclusively, from the peripheral blood from healthy individuals or cancer-bearing patients. The term “T cell” and “T lymphocyte” can be used interchangeably herein. T cells belong to a group of white blood cells known as lymphocytes, and play a central role in cell-mediated immunity. They can be distinguished from other lymphocyte types, such as B cells and natural killer cells, by the presence of a receptor on their cell surface called T cell receptor (TCR). Preferably, the T cell is a human T cell, such as those present in the blood (peripheral blood mononuclear cell, PBMC) or tumor tissue (tumor-infitrating T lymphocytes, TIL).

A T cell is a cell that, optionally after suitable modification, e.g. after being engineered to express a TCR, is capable of producing or mediating an immune response such as a cellular immune response against an epitope to which the TCR is directed. A preferred T cell is a T cell that is enforced or modified to lack endogenous expression of a TCR and which can be modified to express a TCR transgene on the cell surface to enable redirection of T cells to an epitope of interest, i.e., an epitope selectively expressed by cancer cells such as the T cell epitopes disclosed herein.

Several different subsets of T cells have been discovered. T-helper cells assist other white blood cells in immunologic processes, including maturation of B cells into plasma cells and activation of cytotoxic T cells and macrophages, among other functions. These cells are generally known as CD4+ T cells because they express the CD4 protein on their surface, and can phenotypically and functionally distinguished into various subsets, such as T helper type 1, 2, 17 etc. T-helper cells become activated when they are presented with epitopes by MHC class II molecules (also termed HLA-DP, DQ, DR) that are expressed on the surface of antigen presenting cells (APCs). Once activated, T-helper cells divide rapidly and secrete small proteins called cytokines and/or surface express receptors that regulate or assist in the active immune response. Cytotoxic T cells destroy virally infected cells and tumor cells. These cells are also known as CD8+ T cells since they express CD8 at their surface, and can also phenotypically and functionally be distinguished into various subsets, such as T cytotoxic type 1, 2, 17 etc. These cells recognize their targets by binding to epitopes associated with MHC class I molecules (also termed HLA-A, B, C) that are present on the surface of every nucleated cell of the body.

The skilled person can routinely isolate and prepare T cells, usually in vitro or ex vivo, using standard laboratory procedures. For instance, T cells can be isolated from bone marrow, peripheral blood, pieces of tumor of a subject using well known cell separation systems. In embodiments, the T cells are present in a sample of peripheral blood mononuclear cells (PBMC) from a subject. Preferably, the T cell as disclosed herein is generally an activated T cell (e.g. with anti-CD3 and CD28 antibodies), retrovirally transduced with a TCR transgene, and expanded in the presence of cytokines, such as IL-15 and IL-21 (described in Lamers et al., Hum Gene Ther Methods, 25(6):345-357 (2014)).

The term “T cell receptor (TCR)”, as used herein, includes reference to a protein complex that comprises at least two separate peptide chains, which are produced from T cell receptor alpha and beta genes and are called α- and β-TCR chains, and which may naturally complex with CD3 molecules to provide surface expression and function of the TCR. The structure of TCR-ab is similar to immunoglobulin antigen-binding fragment (Fab) fragments, which are composed of a heavy and light chain of the antibody, each consisting of one constant and one variable domain. Each chain of the TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end. The term “variable region of a T cell receptor”, as used herein, includes reference to the variable domain of the TCR chain, and is composed of Variable (V) and Joining (J) segments (in case of TCR alpha, and encoded by the corresponding V and J alpha gene segments, numbered from 1 to 43 and 1-58, respectively) and Variable (V), Diversity (D) and Joining (J) segments (in case of TCR beta, and encoded by the corresponding V beta gene segments, numbered from 1 to 42, combined either with D beta1 (1 gene segment) and J beta1 (6 gene segments) or D beta2 (1 gene segment) and J beta2 (7 gene segments)). The variable region of both the TCR alpha and beta chain have three hypervariable or complementarity determining regions (CDRs) that are recognized for their binding to peptide:MHC complexes (the natural ligands of TCRs). CDR1 and 2 are composed of TCR-V segments (in case of both TCR alpha and beta), whereas CDR3 is composed of fusions of TCR-V, J (in case of TCR alpha) and TCR-V, D, J segments (in case of TCR beta), including nucleotide deletions and insertions. CDR1 and 2 primarily bind MHC itself, and CDR3, being most unique to any TCR, primarily binds the peptide:MHC complex. Preferably, the TCR is a human TCR with or without modifications in the transmembrane and intracellular domains (not affecting the TCR-V domains) to enhance surface expression and/or epitope-specific functions of said TCR (as performed in Govers et al., J Immunol, 2014, 193(10), p. 5315-5326 (2014).

The term “CDR”, as employed herein, relates to “complementary determining region”, which is well known in the art. The CDRs are parts of immunoglobulins or antigen binding receptors (e.g., CARs and TCRs) that determine the specificity of said molecules and make contact with a specific ligand. The CDRs are the most variable part of the molecule and contribute to the antigen binding diversity of these molecules. There are three CDR regions CDR1, CDR2 and CDR3 in each V domain. The CDR regions of an Ig-derived region may be determined as described in “Rabat” (Sequences of Proteins of Immunological Interest”. 5th edit. NIH Publication no. 91-3242 U.S. Department of Health and Human Services (1991); Chothia J. Mol. Biol. 196 (1987), 901-917) or “Chothia” (Nature 342 (1989), 877-883). Preferably a CDR as referred to herein is a (human) T-cell CDR such as a T cell CDR1, a T cell CDR2 or a T cell CDR3.

Preferably, the antigen-binding receptor as referred to herein is a TCR, but does not exclude the use of any other receptor such as an antibody-based receptor. In case of antibody-based receptors, the invention refers particularly to antibody fragments (Fab) or single-chain variable fragments (scFv) with specificity to peptide:MHC complexes (as obtained and described in Chames et al., J Immunol, 169(2), p.1110-1118, 2002). An antibody-based receptor as disclosed herein is preferably a TCR-like antibody, i.e. an antigen-binding receptor that binds to a peptide:MHC complex. Preferably, the antibody-based receptor is a (TCR-like) CAR.

In some aspects and embodiments, the invention provides an engineered T cell, wherein said T cell is engineered to express a (affinity-enhanced) TCR or an antibody-based receptor, such as a CAR, that binds to a T cell epitope of human ropporin-1B (ROPN1B) and/or human ropporin 1-A (ROPN1) as disclosed herein. In embodiments, said antibody-based receptor comprises a binding domain in the form of an antibody fragment (Fab) or single-chain variable fragment (scFv)). In embodiments, said affinity-enhanced TCR or antibody-based receptor does not necessarily comprise (i) CDR1 of SEQ ID NO:12; CDR2 of SEQ ID NO:13 and CDR3 of SEQ ID NO:14; or CDR1 of SEQ ID NO:17; CDR2 of SEQ ID NO:18; and CDR3 of SEQ ID NO:19, (ii) a CDR1 of SEQ ID NO:35; a CDR2 of SEQ ID NO:36; a CDR3 of SEQ ID NO:37; and/or a CDR1 of SEQ ID NO:40; a CDR2 of SEQ ID NO:41; a CDR3 of SEQ ID NO:42, (iii) a CDR1 of SEQ ID NO:48; a CDR2 of SEQ ID NO:49; a CDR3 of SEQ ID NO:50; and/or a CDR1 of SEQ ID NO:53; a CDR2 of SEQ ID NO:54; a CDR3 of SEQ ID NO:55, or (iv) a CDR1 of SEQ ID NO:61; a CDR2 of SEQ ID NO:62; a CDR3 of SEQ ID NO:63; and/or a CDR1 of SEQ ID NO:66; a CDR2 of SEQ ID NO:67; a CDR3 of SEQ ID NO:68. In other words, the aforementioned CDR sequences (e.g. the aforementioned CDR3 sequences) may comprise one, two or three amino acid residue additions, substitutions and/or deletions in order to enhance the affinity of the receptor for the epitope. These variants are also referred to as affinity-enhanced variants and are part of the invention.

The term “binding”, as used herein, includes reference to a binding (interaction) between the “antigen-interaction-site” and the antigen. The term “antigen-interaction-site” defines a motif of a polypeptide which shows the capacity of specific interaction with a specific antigen or a specific group of antigens. Said binding/interaction is also understood to define a “specific recognition”, which, as explained above, is in case of a TCRab definable by the 6 CDR regions (CDR1-3 of TCR alpha and CDR1-3 of TCR beta). The term “specifically recognizing” means in accordance with this invention that the receptor is capable of specifically interacting with and/or binding to a ROPN1 and/or ROPN1B epitope as disclosed herein. The antigen binding moiety of a TCR can recognize, interact and/or bind to different epitopes, albeit with different binding strengths. This relates to the specificity of the TCR, i.e., to its ability to discriminate between the specific regions of an antigenic molecule as disclosed herein. The specific interaction of the antigen-interaction-site with its specific antigen may result in an initiation of an intracellular signal, e.g., due to an oligomerization of the TCR. Thus, a specific motif in the amino acid sequence of the antigen-interaction-site and the antigen bind to each other as a result of their primary, secondary or tertiary structure as well as the result of secondary modifications of said structure. Accordingly, the term “binding to” does not only relate to a linear epitope but may also relate to a conformational epitope, a structural epitope or a discontinuous epitope consisting of two regions of the target molecules or parts thereof. In the context of this invention, a conformational epitope is defined by two or more discrete amino acid sequences separated in the primary sequence which come together on the surface of the molecule when the polypeptide folds to the native protein (Sela, Science 166 (1969), 1365 and Laver, Cell 61 (1990), 553-536). Moreover, the term “binding to” is interchangeably used in the context of the present invention with the term “interacting with”. The ability of the antigen binding moiety of a TCR to bind to a specific target antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Liljeblad et ah, Glyco J 17, 323-329 (2000)), and traditional binding assays (Heeley, Endocr Res 28, 217-229 (2002)). In one embodiment, the extent of binding of a TCR to an unrelated epitope is less than about 10% of the binding of the said TCR to the target epitope (or cognate epitope) as measured, in particular by SPR. In certain embodiments, an antigen binding moiety that binds to the target antigen has a dissociation constant (KD) of ≤1 mM, ≤100 nM, ≤10 nM, ≤1 nM, ≤0.1 nM, ≤0.01 nM, or ≤0.001 nM (e.g., 10-8 M or less, e.g., from 10-8 M to 10-13 M, e.g., from 10-9 M to 10-13 M). The term “specific binding” as used in accordance with the present invention means that the molecules used in the invention do not or do not essentially cross-react with (poly-) peptides of similar structures. Crossreactivity of a panel of TCRs under investigation may be tested, for example, by assessing peptide:MHC binding or epitope-specific responses by TCR-engineered T cells (see Kunert A, J Immunol, 2016, and Kunert A, Clin Cancer Res, 2017) using unrelated peptides and peptides mutated at single amino acid positions as controls. Only those TCRs that bind to the epitope of interest but do not or do not essentially bind to unrelated epitopes are considered specific for the epitope (and thus antigen) of interest and selected for further studies in accordance with the method provided herein. The more amino acid positions are demonstrated to be critical (as based on the analysis of mutated peptides), the more stringent and specific the antigen-binding site of a TCR is. These methods may comprise, inter alia, binding studies, blocking and competition studies with structurally and/or functionally closely related and/or mutated peptides. The binding and functional studies also comprise flow cytometry analysis, surface plasmon resonance (SPR, e.g., with BIAcore®), radiolabeled ligand binding assays and/or stimulation assays using TCR-engineered T cells.

An epitope, also known as antigenic determinant, is the part of an antigen that is recognized by the immune system, specifically by T cells. For example, the epitope is the specific part of the antigen to which a TCR binds. Although epitopes are usually non-self proteins, sequences derived from the host that can be recognized (as in the case of autoimmune diseases or cancer) are also epitopes. Epitopes of protein antigens as described herein may be conformational epitopes or linear epitopes, based on their structure and interaction with the TCR.

The term “binding”, as used herein, includes reference to TCR-peptide:MHC-specific binding, wherein a TCR has binding specificity towards, or has the cap acitity to bind to, a T cell epitope or an antigen comprising said epitope, preferably when the antigen or epitope is presented on an MHC molecule. The skilled person understands that this phrasing does not mean that the TCR is (already) bound to the T cell epitope or target antigen.

The term “antigen”, as used herein, includes reference to an agent comprising an epitope against which an immune response is to be elicited and/or directed. In the present disclosure, an antigen is preferably a proteinaceous molecule which, optionally after processing, induces an immune response, which is specific for the antigen or cells expressing and/or presenting the antigen or its derived epitope. The term “antigen” includes in particular proteins and peptides.

The term “epitope”, as used herein, includes reference to an antigenic determinant in a molecule such as an antigen, i.e., to a part in or fragment of the molecule, for instance a protein, that is recognized by the immune system, for example, that is recognized by a T cell, in particular when presented in the context of an MHC molecule. An epitope of a protein such as human ROPN1 and/or ROPN1B may comprise a continuous or discontinuous portion of said protein and is preferably 8-11 or 15-24 amino acid residues in length when bound to MHC class I or II, respectively. For instance, the epitope can be 5, 6, 7, 8, 9, 10, 11, 12, 13, 14, 15, 16, 17, 18,19, 20, 21, 22, 23 or 24 amino acid residues in length. The epitope as disclosed herein herein is preferably a T cell epitope.

The term “ROPN1 and/or ROPN1B”, as used herein, includes reference to a protein that is associated with male fertility. ROPN1 (also referred to as ropporin-1A herein) and/or ROPN1B (also referred to as ropporin-1B herein) is involved amongst other functions in the regulation of fibrous sheath integrity and sperm motility, and plays a role in PKA-dependent signaling processes required for spermatozoa capacitation. Preferably, in the present disclosure, ROPN1 and/or ROPN1B is human ROPN1 and ROPN1B, and preferred examples are human ROPN1A/ROPN1 and its isoform ROPN1B. The amino acid sequence of human ROPN1 is accessible under UniProtKB Acc. No. Q9HATO-1 (Last modified: Oct. 1, 2001-v2). The amino acid sequence of human ROPN1B is accessible under UniProtKB Acc. No. Q9BZX4-1 (Last modified:Jun. 1, 2001-v1). All of the identified T cell epitopes (Tables 1-4, SEQ ID NOs:1-9, 20, 23-32, 43 and 56) are present in the amino acid sequence of human ROPN1 and/or ROPN1B. Some of the identified T cell epitopes are exclusively present in the amino acid sequence of human ROPN1B and are not present in the amino acid sequence of human ROPN1, and vice versa.

The term “immune response”, as used herein, includes reference to an integrated bodily response to an antigen and preferably refers to a cellular immune response or a cellular as well as a humoral immune response. An immune response may be protective/preventive/prophylactic and/or therapeutic.

The term “cellular immune response”, as used herein, includes reference to a cellular response directed to cells that present an (epitope of an) antigen in the context of MHC class I or class II.

Preferably, in the medical methods as disclosed herein, the target of an immune response is a cell (i.e., target cell), preferably a tumor or cancer cell, that expresses human ROPN1 and/or ROPN1B. Preferably, said cell is in a subject. For instance, said cell is a cell that expresses human ROPN1 and/or ROPN1B and which displays or presents on its cell surface T cell epitopes in complex with an MHC molecule, such as an MHC class I molecule (for instance an HLA-A, such as HLA-A*02, molecule), preferably wherein said T cell epitope is one of SEQ ID NOs:1-9, 20, 23-32, 43 and 56, preferably one of SEQ ID NOs:4, 23, 24, 43 or 56, more preferably one of SEQ ID NOs: 4, 23 or 24, even more preferably SEQ ID NO:4.

The term “Major Histocompatibility Complex” and the corresponding abbreviation “MHC”, as used herein, includes reference to MHC class I and MHC class II molecules. MHC molecules relate to a complex of genes that occurs in all vertebrates. MHC molecules are important proteins that enable recognition of antigen presenting cells or diseased cells by T cells in immune reactions, and the activation of the T cells. MHC molecules bind epitopes, such as peptides, and present them for recognition by TCRs. The proteins encoded by the MHC are expressed on the surface of cells, and display both self antigens (peptide fragments from the cell itself) and nonself antigens (e.g., fragments of invading microorganisms or aberrant molecules that once were self antigens) to a T cell. The MHC molecules are divided into three subgroups, class I, class II, and class III. MHC class I proteins are generally known to present antigenic determinants to cytotoxic T cells. Generally, MHC class II proteins are known to present antigenic determinants to T-helper cells. In humans, MHC genes are often referred to as human leukocyte antigen (HLA) genes, and MHC molecules are often referred to as HLA molecules. HLA genes encode nine classical groups: HLA-A, HLA-B, HLA-C, HLA-DPA1, HLA-DPB1, HLA-DQA1, HLA-DQB1, HLA-DRA, and HLA-DRB1.

Preferably, in the present disclosure, an MHC molecule is an HLA molecule. In embodiments, the HLA molecule (protein) is an HLA-A molecule, more preferably an HLA-A*02 molecule. Preferably, a TCR as disclosed herein is a TCR that binds to an HLA-A molecule, more preferably an HLA-A*02 molecule.

The term “collection”, as used herein in relation to T cells, includes reference to a set of T cells that are engineered to express the same TCR as disclosed herein or a different TCR as disclosed herein. For instance, the collection may comprise T cells that are engineered to express a TCR that binds to the epitope of SEQ ID NO:1 or SEQ ID NO:20 and said collection may also comprise T cells that are engineered to express a TCR that binds to the epitope of SEQ ID NO:2, etc. As the skilled person appreciates, a collection of T cells can be administered in the form of a pharmaceutical composition that additionally contains a pharmaceutically acceptable excipient such as a pharmaceutically acceptable carrier or diluent.

The term “tumor”, as used herein, includes reference to abnormal cellular growth that can be benign, pre-cancerous, malignant, or metastatic. Preferably, the tumor is a malignant neoplasm, i.e. a cancer. The tumor can be a solid tumor such as a carcinoma or a blood (liquid) tumor such as a lymphoma, myeloma or leukemia. Preferably, the tumor is a solid tumor, more preferably the tumor is a solid tumor characterized by tumor cells expressing human ROPN1 and/or ROPN1B, preferably, and in the context of the disclosed TCR, human ROPN1 or ROPN1B. In preferred embodiments, said solid tumor is a breast cancer, for instance TNBC, or a skin cancer, such as a melanoma more preferably a skin cutaneous melanoma (SKCM).

As used herein, the term “cancer” includes (but is not limited to) reference to cancers characterized by the presence of a cancer cell selected from the group consisting of a cell of an adrenal gland tumor, an AIDS-associated cancer, an alveolar soft part sarcoma, an astrocytic tumor, bladder cancer (squamous cell carcinoma and transitional cell carcinoma), bone cancer (adamantinoma, aneurismal bone cysts, osteochondroma, osteosarcoma), a brain and spinal cord cancer (glioma), a metastatic brain tumor, a breast cancer, a carotid body tumor, a cervical cancer, a chondrosarcoma, a dhordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer (nephroblastoma, papillary renal cell carcinoma), a leukemia, a lipoma/benign lipomatous tumor, a liposarcoma/malignant lipomatous tumor, a liver cancer (hepatoblastoma, hepatocellular carcinoma), a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious unveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer (carcinoma of the cervix, endometrial carcinoma, and leiomyoma), or any other malignant tissue.

The term “subject” or “patient”, as used herein, includes reference to an individual who is suffering, or suspected of suffering, from a tumor. In other words, the term “subject” or “patient” can be used to indicate an individual who has a tumor such as a cancer. Preferably, the subject is a mammal, more preferably a primate, most preferably a human.

The term “nucleic acid”, as used herein, includes reference to DNA and RNA including mRNA or cDNA, as well as synthetic congeners thereof. The nucleic acid can be a natural, recombinant or synthetic nucleic acid.

The term “amino acid”, as used herein, includes reference to naturally occurring monomers of a protein, as well as synthetic congeners thereof. An amino acid residue can be a natural, recombinant or synthetic amino acid residue.

The term “% sequence identity”, as used herein, includes reference to the percentage of nucleotides in a nucleic acid sequence, or amino acid residues in an amino acid sequence, that is identical with the nucleotides, resp. amino acid residues, in a nucleic acid or amino acid sequence of interest, after aligning the sequences and optionally introducing gaps, if necessary, to achieve the maximum percent sequence identity. Methods and computer programs for alignments are well known in the art. Sequence identity is calculated over substantially the whole length, preferably the whole (full) length, of an amino acid sequence of interest. The skilled person understands that consecutive amino acid residues in one amino acid sequence are compared to consecutive amino acid residues in another amino acid sequence.

T cell epitopes The present inventors discovered a set of human ropporin-1B (ROPN1B) and/or human ropporin-1A (ROPN1) T cell epitopes that can be used as targets of TCR-engineered T cells as disclosed herein. This set of human ropporin-1B (ROPN1B) and/or human ropporin-1A (ROPN1) T cell epitopes is listed in Tables 1-4 and as SEQ ID NOs:1-9, 20, 23-32, 43 and 56, and form part of the present invention. Preferred T cell epitopes are identified by SEQ ID NOs:4 (FLY-A epitope), 23 (FLY-B epitope), 24 (EVI epitope), 43 or 56, more preferably one of SEQ ID NOs: 4, 23 or 24, even more preferably SEQ ID NO:4.

The invention thus provides an isolated or purified peptide of human ropporin-1B (ROPN1B) and/or human ropporin-1A (ROPN1), which forms a complex with a human Major Histocompatibility Complex (MHC) molecule, preferably an HLA-A*02 molecule, wherein said peptide consists of the amino acid sequence of any one of SEQ ID NOs:1-9, 20, 23-32, 43 and 56.

In some embodiments, the peptide consists of the amino acid sequence of any one of SEQ ID NOs:1-9, 20, 23-32, 43 and 56, or sequences having at least 70% or at least 80% sequence identity thereto. In embodiments, the peptide consists of (i) a modified amino acid sequence of SEQ ID NO:1 in which the amino acid residue at position 6 (Glu), position 8 (Glu) and/or position 9 (Val) of SEQ ID NO:1 is substituted by another amino acid residue, (ii) a modified amino acid sequence of SEQ ID NO:4 in which the amino acid residue at position 1 (F), position 2 (L) and/or position 9 (V) of SEQ ID NO:4 is substituted by another amino acid residue, (iii) a modified amino acid sequence of SEQ ID NO:23 in which the amino acid residue at position 1 (F) and/or position 9 (V) of SEQ ID NO:23 is substituted by another amino acid residue.

In the same context, the invention also provides an isolated or synthesized human MHC molecule in complex with a peptide (T cell epitope) of human ropporin-1B (ROPN1B) and/or human ropporin-1A (ROPN1) of the invention.

The invention also provides a use of (i) a human ropporin-1B (ROPN1B) and/or human ropporin-1A (ROPN1) T cell epitope as disclosed herein or (ii) a human MHC molecule in complex with a human ropporin-1B (ROPN1B) and/or human ropporin-1A (ROPN1) T cell epitope as disclosed herein, for identifying, screening, purifying, enriching and/or affinity maturating of T-cells.

Summary of Steps to Select and Validate ROPN1 and/or ROPN1B-Derived T Cell Epitopes

The ROPN1B protein consists of 210 amino acids and has 95% sequence overlap with ROPN1. The vast majority of MHC-I peptides are 8-11 amino acids in length, which results in a total of 814 theoretical epitopes that could originate from ROPN1 and/or ROPN1B.

In the field of tumor immunology, most studies select T cell epitopes according to a single in silico prediction tool. In contrast, the inventors selected unique ROPN1 and/or ROPN1B T cell epitopes by using multiple in silico tools (with self-devised cut offs), combined with immunopeptidome analysis of MHC-I eluted epitopes. The total collection of epitopes was filtered for non-homology to any epitope present in other proteins, and subsequently validated in vitro for MHC-I binding as well as immunogenicity using HLA-A2-expressing cells and stimulation of naive T cells from healthy donors.

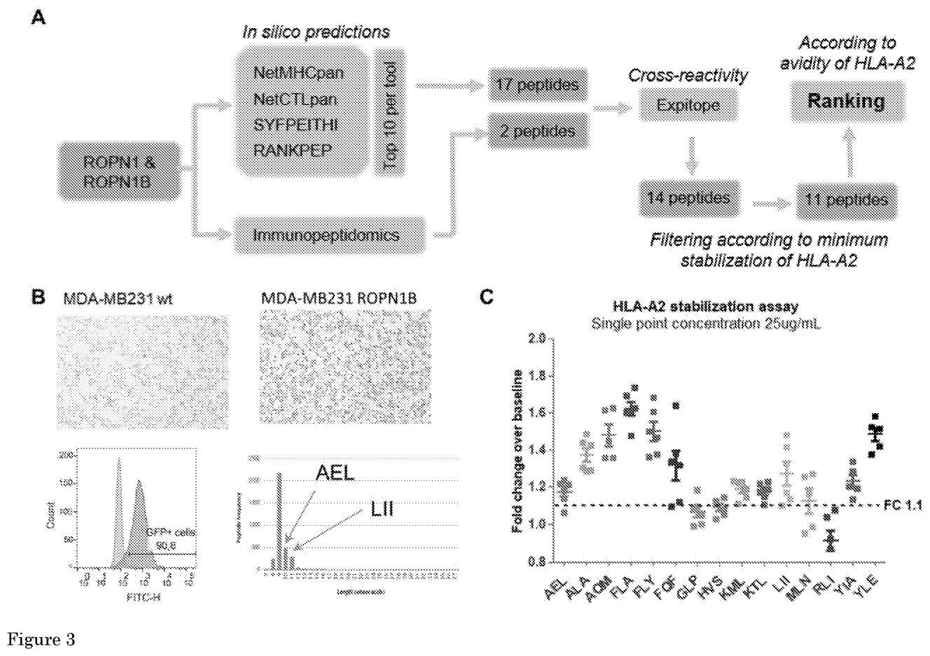

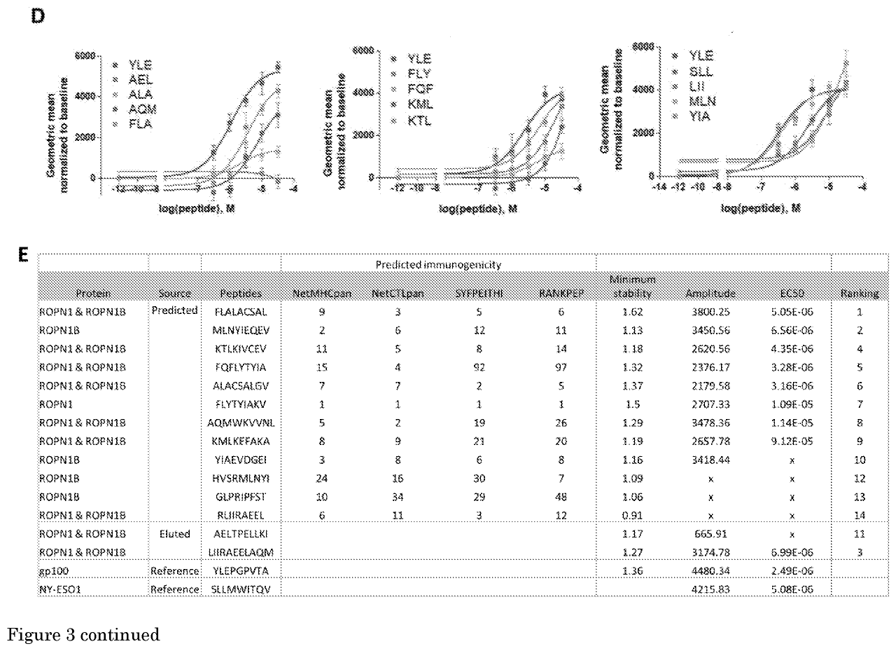

In more detail, the selection and validation of ROPN1 and/or ROPN1B epitopes followed the following 6 steps.
1. prediction using multiple publicly available tools for epitope presentation by HLA-A2:01, which, based on cut-offs, yielded n=17 immunogenic epitopes (see FIG. 3 for details).
2. immunopeptidome analysis (i.e., mass-spectrometry analysis of MHC-I bound peptides from HLA-A2-expressing cancer cell line), which yielded n=2 additional epitopes (11-mer epitope with predicted binding to HLA-A2:01, and a 10-mer epitope with predicted binding to HLA-B40:01).
3. filtering of epitopes for non-homology (>2 amino acids difference to any peptide sequence not derived from ROPN1 and/or ROPN1B), which narrowed down the number of non-cross-reactive epitopes to 14.
4. assessment of ability to bind to HLA-A2:01, which, using our threshold of minimum HLA-A2 binding, narrowed down the number of epitopes to 11.
5. assessment of strength (affinity) to bind to HLA-A2:01, which is an important feature to elicit a T cell response and narrowed down the number of epitopes to 9 (see FIG. 3E, Table 2, and SEQ ID NOs:1-9).
6. assessment of ability to enrich epitope-specific T cells derived from healthy donors, which is considered a measure of the epitope's immunogenicity. Thus far, T cell responses were observed against all epitopes tested in co-cultures of antigen presenting cells that are loaded with peptides together with autologous naive T cells derived from healthy donor PBMC (Table 2).

Further steps for selection and validation of T cell epitopes and T cell receptors are provided in Example 2 and FIGS. 7-14.

### Identification and Selection of TCRs

Following the identification of ROPN1 and/or ROPN1B as a target antigen for adoptive T cell therapy, and their epitopes as disclosed herein, one of skill will understand how to prepare cells that express a ROPN1 and/or ROPN1B epitope which can subsequently be used to generate and/or enrich for host T cells comprising a ROPN1 and/or ROPN1B epitope-specific TCR. Detailes of such methods are described in the experimental section hereinbelow.

Briefly, epitope-specific T cells can be isolated form healthy donor blood or solid cancer patients' blood or tumor tissue via staining and sorting with said epitope in compex with fluorescent-labeled HLA-molecules or via staining and sorting with anti-IFNg and magnetically-labeled capture antibodies. Such T cells, prior to above staining and isolation, can be enriched upon co-culture with artificial or autologous antigen presenting cells, such as dendritic cells (CD11c+) or genetically modified B cells, such as K562 cells. Reference is als made to Example 1 and Example 2, Materials and Methods for further details and references.

### TCR-Engineered T Cells

Engineered cells in the context of the present invention are immune cells such as T cells or NK cells, but preferably are T cells. Generation of tumour antigen-specific T cells according to the present invention, which preferably have the specificity and capacity to kill tumour cells, may be performed by employing one or more of different strategies generally known in the art.

In one embodiment, tumour-reactive host T cells may be identified and selected as described above and grown out of a population of peripheral blood mononuclear cells (PBMCs) or tumour infiltrating lymphocytes (TILs). Once such cells have been generated and isolated, they may be expanded for use. In one embodiment, such tumour-reactive host T cells are investigated to reveal the nucleic acid sequence of their cancer antigen-specific TCR.

Alternatively or successively, in the same or another embodiment, host T cells can be modified to become tumour reactive by genetically modifying a host T cell to express one or more tumour-specific TCRs as disclosed herein, or as identified by using the method of TCR selection described above. Such genetic modification may occur by transfection or transduction, preferably by transduction, such as by using retroviral technology.

Host T cells in the context of this invention are preferably human T cells, more preferably human CD8+ T cells, and may be autologous or allogeneic host cells, preferably autologous cells.

As used herein, “autologous” refers to genetically identical cells derived from the same donor, e.g. cells obtained from the patient are processed to target the cancer and the cells are then administered back to the patient's body, whereas the term “allogeneic” refers to cells derived from a genetically non-identical donor.

The genetic modification of cells can be accomplished by transducing the cells, preferably a substantially homogeneous composition of cells, with a recombinant DNA or RNA construct encoding an antigen-binding receptor such as a TCR or antibody-based receptor as disclosed herein, preferably a TCR. A vector, preferably, a retroviral vector (either gamma retroviral or lentiviral) can be employed for the introduction of the recombinant DNA or RNA construct encoding a TCR into the host cell genome. For example, a polynucleotide encoding a TCR that binds an epitope of ROPN1 and/or ROPN1B as described herein can be cloned into a retroviral vector and expression can be driven from its endogenous promoter, from the retroviral long terminal repeat, or from an alternative internal promoter. Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs)), or transgene expression (e.g., using a natural or chemically modified RNA) can be used.

Preferably, the engineered cells are modified with a nucleic acid construct that contains a promotor nucleic acid sequence that regulates the expression of a TCR or antibody-based receptor as disclosed herein, wherein said promotor is operably linked to a nucleic acid that encodes a TCR as disclosed herein.

For genetic modification of the cells to provide tumor antigen-specific cells, a retroviral vector is preferably employed, however any other suitable viral vector or non-viral delivery system can be used for transduction of the cell with the tumour-antigen reactive TCR. Preferably, the chosen vector exhibits high efficiency of infection and stable integration and expression. Other viral vectors that can be used include, for example, adenoviral, lentiviral, and adeno-associated viral vectors, vaccinia virus, a bovine papilloma virus, or a herpes virus, such as Epstein-Barr Virus. Retroviral vectors are particularly well developed and have been used in clinical settings for decades.

Non-viral approaches can also be employed for the expression of a protein in a cell. For example, a nucleic acid molecule can be introduced into a cell by transfection, e.g. by administering the nucleic acid in the presence of lipofection, asialoorosomucoid-polylysine conjugation, or by micro-injection under surgical condition, all of which are known by the art. Other non-viral means for gene transfer include transfection in vitro using calcium phosphate, DEAE dextran, electroporation, and protoplast fusion. Liposomes can also be potentially beneficial for delivery of DNA or RNA into a cell. Recombinant receptors can also be derived or obtained using transposases or targeted nucleases (e.g., transcription activator-like effector nucleases (TALENs), Zinc-finger nucleases (ZFNs), and/or clustered regularly interspaced short palindromic repeats (CRISPRs)). Transient expression may be obtained by RNA electroporation.

The resulting modified cells can be grown under conditions similar to those for unmodified cells, whereby the modified cells can be expanded to provide T cells in accordance with the present invention which can be used for therapy.

In the present invention, functional variants of the TCRs as disclosed herein are also envisaged, such as TCRs in which a CDR1, a CDR2 and/or a CDR3 (for instance a CDR1 and a CDR2; a CDR1 and a CDR3; a CDR2 and a CDR3; or a CDR1, a CDR2 and a CDR3) as disclosed herein is/are modified or changed in that 1, 2, 3, 4 or 5 amino acid residues are added, substituted and/or deleted while at least maintaining (or improving) the binding specificity and/or binding properties (of said CDR and/or of a TCR comprising said CDR).

In the same manner, in relation to a T cell receptor that comprises a VDJ, a VJ, an alpha chain and/or a beta chain amino acid sequence as disclosed herein (such as SEQ ID NO:11, SEQ ID NO:16, SEQ ID NO:21, SEQ ID NO:22, SEQ ID NO:34, SEQ ID NO:39, SEQ ID NO:44, SEQ ID NO:45, SEQ ID NO:47, SEQ ID NO:52, SEQ ID NO:57, SEQ ID NO:58, SEQ ID NO:60, SEQ ID NO:65, SEQ ID NO:69 and SEQ ID NO:70), functional variants thereof are envisaged herein that have at least 70%, 80% or at least 90% sequence identity to said VDJ, said VJ, said alpha chain and/or said beta chain amino acid sequences as disclosed herein while at least maintaining (or improving) the binding specificity and/or binding properties (of a TCR that comprises said VDJ, said VJ, said alpha chain and/or said beta chain amino acid sequences as disclosed herein).

For instance, the present invention provides an engineered T cell, wherein said T cell is engineered to express a T cell receptor (TCR) that binds to a T cell epitope of human ropporin-1A (ROPN1) and/or human ropporin-1B (ROPN1B), wherein said TCR comprises: (i) a T cell receptor alpha chain comprising a hypervariable region that comprises a CDR3 of SEQ ID NO:14, SEQ ID NO:37, SEQ ID NO:50 or SEQ ID NO:63 (preferably SEQ ID NO:37, SEQ ID NO:50 or SEQ ID NO:63) or a functional variant thereof, for instance a variant of SEQ ID NO:14, SEQ ID NO:37, SEQ ID NO:50 or SEQ ID NO:63 in which 1, 2, 3, 4 or 5 amino acid residues are added, substituted and/or deleted while at least maintaining (or improving) the binding specificity and/or binding properties (of said CDR3 of SEQ ID NO:14, SEQ ID NO:37, SEQ ID NO:50 or SEQ ID NO:63 and/or of a TCR comprising said CDR3 of SEQ ID NO:14, SEQ ID NO:37, SEQ ID NO:50 or SEQ ID NO:63), and/or (ii) a T cell receptor beta chain comprising a hypervariable region that comprises a CDR3 of SEQ ID NO:19, SEQ ID NO:42, SEQ ID NO:55 or SEQ ID NO:68 (preferably SEQ ID NO:42, SEQ ID NO:55 or SEQ ID NO:68) or a functional variant thereof, for instance a variant of SEQ ID NO:19, SEQ ID NO:42, SEQ ID NO:55 or SEQ ID NO:68 in which 1, 2, 3, 4 or 5 amino acid residues are added, substituted and/or deleted while at least maintaining (or improving) the binding specificity and/or binding properties (of said CDR3 of SEQ ID NO:19, SEQ ID NO:42, SEQ ID NO:55 or SEQ ID NO:68 and/or of a TCR comprising said CDR3 of SEQ ID NO:19, SEQ ID NO:42, SEQ ID NO:55 or SEQ ID NO:68; wherein said hypervariable regions of said T cell receptor alpha and beta chain further comprise a CDR1 and a CDR2.

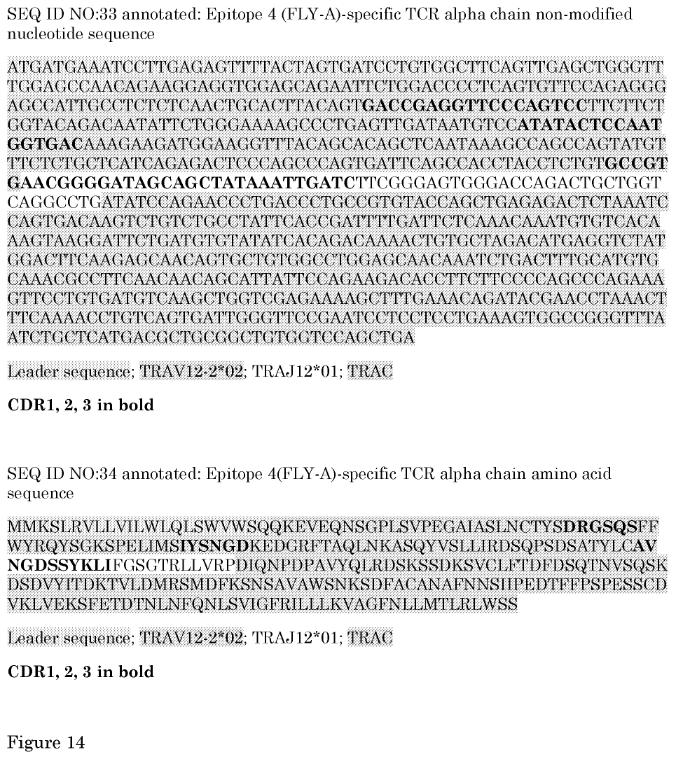

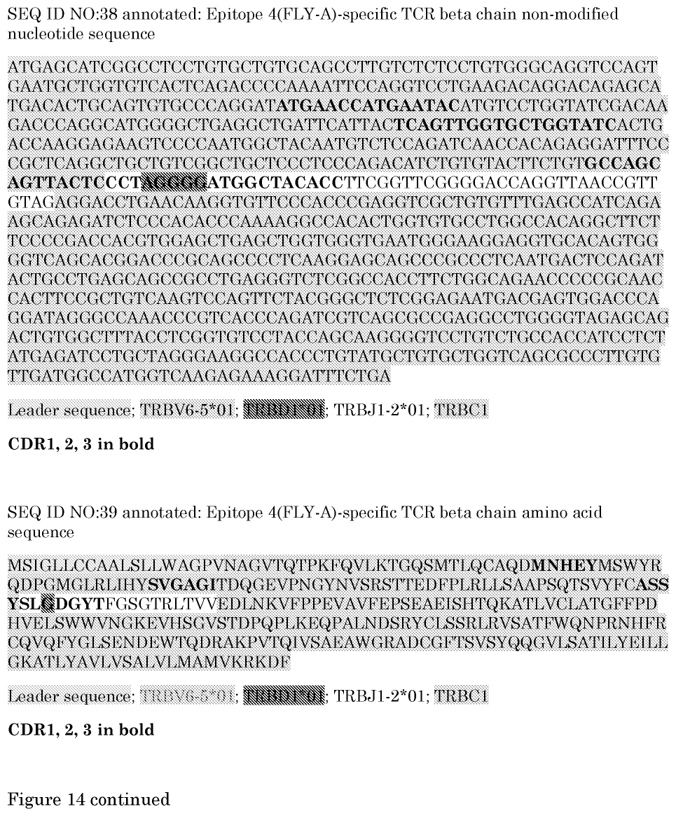

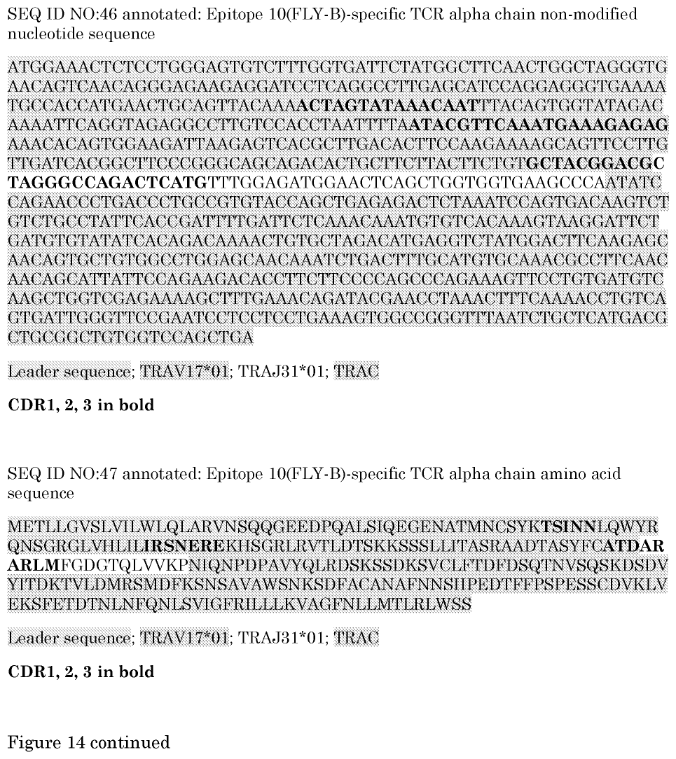

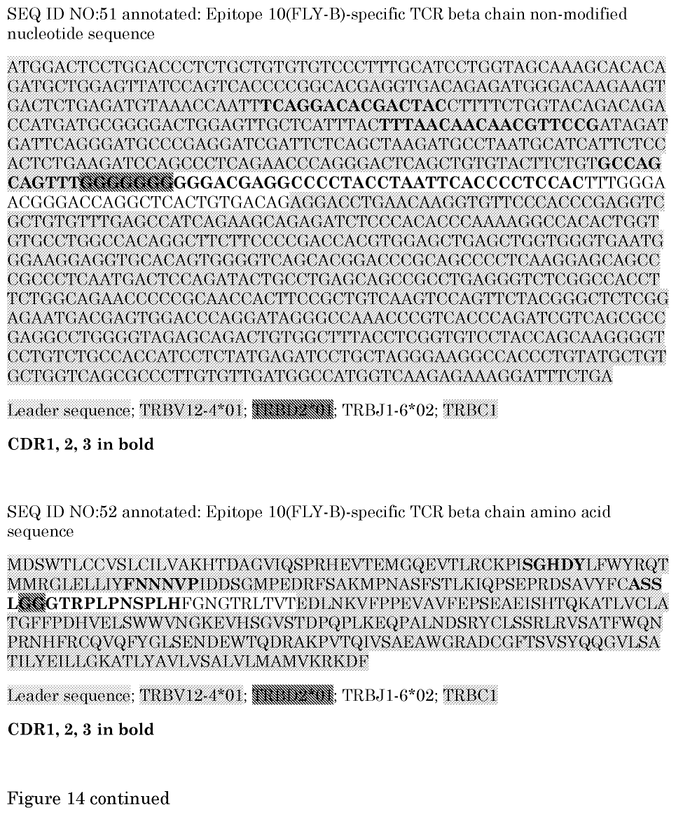

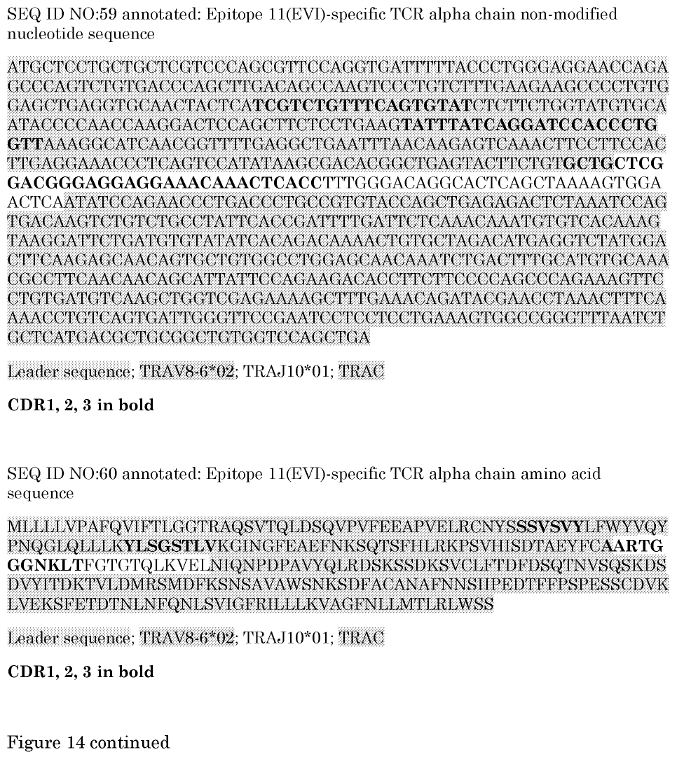

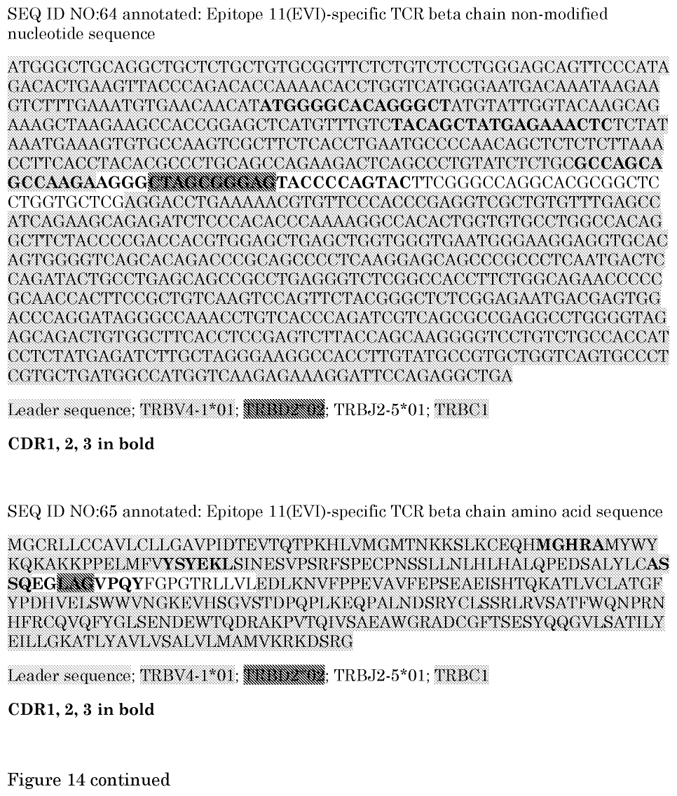

The skilled person routinely understands from amongst others FIGS. 6 and 14 which CDR combinations, and thus their functional variants, belong together.

In the same manner, the invention provides for instance an engineered T cell, wherein said T cell is engineered to express a T cell receptor (TCR) that binds to a T cell epitope of human ropporin-1A (ROPN1) and/or human ropporin-1B (ROPN1B), wherein said hypervariable region of said T cell receptor comprises:
a CDR1 of SEQ ID NO:12, SEQ ID NO:35, SEQ ID NO:48 or SEQ ID NO:61 (preferably SEQ ID NO:35, SEQ ID NO:48 or SEQ ID NO:61) or a functional variant thereof, for instance a variant of SEQ ID NO:12, SEQ ID NO:35, SEQ ID NO:48 or SEQ ID NO:61 in which 1, 2, 3, 4 or 5 amino acid residues are added, substituted and/or deleted while at least maintaining (or improving) the binding specificity and/or binding properties (of said CDR1 of SEQ ID NO:12, SEQ ID NO:35, SEQ ID NO:48 or SEQ ID NO:61 and/or of a TCR comprising said CDR1 of SEQ ID NO:12, SEQ ID NO:35, SEQ ID NO:48 or SEQ ID NO:61);
a CDR2 of SEQ ID NO:13, SEQ ID NO:36, SEQ ID NO:49 or SEQ ID NO:62 (preferably SEQ ID NO:36, SEQ ID NO:49 or SEQ ID NO:62) or a functional variant thereof, for instance a variant of SEQ ID NO:13, SEQ ID NO:36, SEQ ID NO:49 or SEQ ID NO:62 in which 1, 2, 3, 4 or 5 amino acid residues are added, substituted and/or deleted while at least maintaining (or improving) the binding specificity and/or binding properties (of said CDR2 of SEQ ID NO:13, SEQ ID NO:36, SEQ ID NO:49 or SEQ ID NO:62 and/or of a TCR comprising said CDR2 of SEQ ID NO:13, SEQ ID NO:36, SEQ ID NO:49 or SEQ ID NO:62);
a CDR3 of SEQ ID NO:14, SEQ ID NO:37, SEQ ID NO:50 or SEQ ID NO:63 (preferably SEQ ID NO:37, SEQ ID NO:50 or SEQ ID NO:63) or a functional variant thereof, for instance a variant of SEQ ID NO:14, SEQ ID NO:37, SEQ ID NO:50 or SEQ ID NO:63 in which 1, 2, 3, 4 or 5 amino acid residues are added, substituted and/or deleted while at least maintaining (or improving) the binding specificity and/or binding properties (of said CDR3 of SEQ ID NO:14, SEQ ID NO:37, SEQ ID NO:50 or SEQ ID NO:63 and/or of a TCR comprising said CDR3 of SEQ ID NO:14, SEQ ID NO:37, SEQ ID NO:50 or SEQ ID NO:63); and/or
   wherein said hypervariable region of said T cell receptor beta chain comprises:
a CDR1 of SEQ ID NO:17, SEQ ID NO:40, SEQ ID NO:53 or SEQ ID NO:66 (preferably SEQ ID NO:40, SEQ ID NO:53 or SEQ ID NO:66) or a functional variant thereof, for instance a variant of SEQ ID NO:17, SEQ ID NO:40, SEQ ID NO:53 or SEQ ID NO:66 in which 1, 2, 3, 4 or 5 amino acid residues are added, substituted and/or deleted while at least maintaining (or improving) the binding specificity and/or binding properties (of said CDR1 of SEQ ID NO:17, SEQ ID NO:40, SEQ ID NO:53 or SEQ ID NO:66 and/or of a TCR comprising said CDR1 of SEQ ID NO:17, SEQ ID NO:40, SEQ ID NO:53 or SEQ ID NO:66);
a CDR2 of SEQ ID NO:18, SEQ ID NO:41, SEQ ID NO:54 or SEQ ID NO:67 (preferably SEQ ID NO:41, SEQ ID NO:54 or SEQ ID NO:67) or a functional variant thereof, for instance a variant of SEQ ID NO:18, SEQ ID NO:41, SEQ ID NO:54 or SEQ ID NO:67 in which 1, 2, 3, 4 or 5 amino acid residues are added, substituted and/or deleted while at least maintaining (or improving) the binding specificity and/or binding properties (of said CDR2 of SEQ ID NO:18, SEQ ID NO:41, SEQ ID NO:54 or SEQ ID NO:67 and/or of a TCR comprising said CDR2 of SEQ ID NO:18, SEQ ID NO:41, SEQ ID NO:54 or SEQ ID NO:67); and
a CDR3 of SEQ ID NO:19, SEQ ID NO:42, SEQ ID NO:55 or SEQ ID NO:68 (preferably SEQ ID NO:42, SEQ ID NO:55 or SEQ ID NO:68) or a functional variant thereof, for instance a variant of SEQ ID NO:19, SEQ ID NO:42, SEQ ID NO:55 or SEQ ID NO:68 in which 1, 2, 3, 4 or 5 amino acid residues are added, substituted and/or deleted while at least maintaining (or improving) the binding specificity and/or binding properties (of said CDR3 of SEQ ID NO:19, SEQ ID NO:42, SEQ ID NO:55 or SEQ ID NO:68 and/or of a TCR comprising said CDR3 of SEQ ID NO:19, SEQ ID NO:42, SEQ ID NO:55 or SEQ ID NO:68).

In the same manner, the invention provides an engineered T cell, wherein said T cell is engineered to express a T cell receptor (TCR) that binds to a T cell epitope of human ropporin-1A (ROPN1) and/or human ropporin-1B (ROPN1B), wherein said TCR comprises:
(i) a T cell receptor alpha chain that comprises an amino acid sequence of SEQ ID NO:21, SEQ ID NO:44, SEQ ID NO:57 or SEQ ID NO:69 (preferably SEQ ID NO:44, SEQ ID NO:57 or SEQ ID NO:69) or a functional variant thereof that has at least 70%, at least 80% or at least 90% sequence identity with SEQ ID NO:21, SEQ ID NO:44, SEQ ID NO:57 or SEQ ID NO:69 (preferably SEQ ID NO:44, SEQ ID NO:57 or SEQ ID NO:69) while at least maintaining (or improving) the binding specificity and/or binding properties (of the protein of SEQ ID NO:21, SEQ ID NO:44, SEQ ID NO:57 or SEQ ID NO:69 and/or of a TCR comprising the amino acid sequence of SEQ ID NO:21, SEQ ID NO:44, SEQ ID NO:57 or SEQ ID NO:69); or a T cell receptor alpha chain that comprises an amino acid sequence of SEQ ID NO:11, SEQ ID NO:34, SEQ ID NO:47 or SEQ ID NO:60 (preferably SEQ ID NO:34, SEQ ID NO:47 or SEQ ID NO:60) or a functional variant thereof that has at least 70%, at least 80% or at least 90% sequence identity with SEQ ID NO:11, SEQ ID NO:34, SEQ ID NO:47 or SEQ ID NO:60 while at least maintaining (or improving) the binding specificity and/or binding properties (of the protein of SEQ ID NO:11, SEQ ID NO:34, SEQ ID NO:47 or SEQ ID NO:60 and/or of a TCR comprising the amino acid sequence of SEQ ID NO:11, SEQ ID NO:34, SEQ ID NO:47 or SEQ ID NO:60); and/or
(ii) a T cell receptor beta chain that comprises an amino acid sequence of SEQ ID NO:22, SEQ ID NO:45, SEQ ID NO:58 or SEQ ID NO:70 (preferably SEQ ID NO:45, SEQ ID NO:58 or SEQ ID NO:70) or a functional variant thereof that has at least 70%, at least 80% or at least 90% sequence identity with SEQ ID NO:22, SEQ ID NO:45, SEQ ID NO:58 or SEQ ID NO:70 (preferably SEQ ID NO:45, SEQ ID NO:58 or SEQ ID NO:70) while at least maintaining (or improving) the binding specificity and/or binding properties (of the protein of SEQ ID NO:22, SEQ ID NO:45, SEQ ID NO:58 or SEQ ID NO:70 and/or of a TCR comprising the amino acid sequence of SEQ ID NO:22, SEQ ID NO:45, SEQ ID NO:58 or SEQ ID NO:70); or a T cell receptor beta chain that comprises an amino acid sequence of SEQ ID NO:16, SEQ ID NO:39, SEQ ID NO:52 or SEQ ID NO:65 (preferably SEQ ID NO:39, SEQ ID NO:52 or SEQ ID NO:65) or a functional variant thereof that has at least 70%, at least 80% or at least 90% sequence identity with SEQ ID NO:16, SEQ ID NO:39, SEQ ID NO:52 or SEQ ID NO:65 while at least maintaining (or improving) the binding specificity and/or binding properties (of the protein of SEQ ID NO:16, SEQ ID NO:39, SEQ ID NO:52 or SEQ ID NO:65 and/or of a TCR comprising the amino acid sequence of SEQ ID NO:16, SEQ ID NO:39, SEQ ID NO:52 or SEQ ID NO:65).

### Treatment

The invention further provides a T cell as disclosed herein, wherein said T cell is for use in therapy. Preferably, the T cell is for use in the treatment of a solid or liquid tumor, preferably a cancer, in a subject.

In the same manner, the invention provides a method of treating a subject suffering, or suspected of suffering, from a solid or liquid tumor, comprising the step of:—administering a therapeutically effective amount of a T cell as disclosed herein to said subject. The invention also provides a method for binding a T cell as disclosed herein to a T cell epitope as disclosed herein in a subject suffering, or suspected of suffering, from a solid or liquid tumor, comprising the step of: administering a T cell as disclosed herein to said subject.

In the same manner, the invention provides a use of a T cell as disclosed herein in the manufacture of a medicament for the treatment of a solid or liquid tumor in a subject.

The term “therapeutically effective amount”, as used herein, includes reference to an amount of a T cell that, when administered as part of a desired dosage regimen (to a mammal, preferably a human) alleviates a symptom, ameliorates a condition, or slows the onset of disease conditions according to clinically acceptable standards for the disorder or condition to be treated, e.g. at a reasonable benefit/risk ratio applicable to any medical treatment. The precise effective amount for a subject will depend upon the subject's size and health, the nature and extent of the condition, and can be determined by the skilled person in a routine manner.

The terms “treating” and “treatment”, as used herein, include reference to reversing, reducing, and/or arresting the symptoms, clinical signs, and/or underlying pathology of a condition with the goal to improve or stabilize a subject's condition.

In embodiments, the solid tumor comprises tumor cells expressing human ROPN1 and/or ROPN1B. As is apparent from the present disclosure, the T cells and TCRs as disclosed herein specifically bind to human ROPN1 and/or ROPN1B, preferably human ROPN1B, T cell epitopes.

Two preferred examples of solid tumors that comprise tumor cells expressing human ROPN1 and/or ROPN1B, preferably human ROPN1B, are breast cancer, for instance triple negative breast cancer (TNBC), and skin cancer, for instance a melanoma such as skin cutaneous melanoma (SKCM). Preferably, the subject suffers, or is suspected of suffering, from a triple negative breast cancer or a melanoma such as skin cutaneous melanoma (SKCM).

Triple negative breast cancer (TNBC) is an aggressive breast cancer subtype, accounting for 15-20% of all breast cancer (BC) cases. TNBC is characterized by the absence of receptors for estrogen and progesterone and lack of human-epidermal growth factor receptor 2 (HER2), and is therefore not responding to current hormone receptor or HER2-targeting therapies. Despite recent approval of immune checkpoint inhibitors (ICI) for PD-L1-positive TNBC, the majority of patients does not respond to this treatment.

Preferably, the T cells as disclosed herein are for use in adoptive T cell therapy such as therapy with TCR-engineered T cells. Adoptive T cell therapy involves the isolation of T cells from a subject and in vitro or ex vivo expansion of said T cells. The T cells are then infused into a patient with a tumor in an attempt to give the immune system the ability to overwhelm remaining tumor via T cells which can attack and kill cancer. There are muliple forms of T cells to be used for adoptive T cell therapy to treat cancer; tumor infiltrating lymphocytes (TIL), particular T cell or clone, and T cells that have been engineered to recognize and attack tumors.

Preferably, the T cells as disclosed herein are autologous T cells and are for autologous T cell therapy. In this context, autologous means that the T cells are obtained from the subject that is to be treated.

Engineered T cell receptor (TCR) T cell therapy involves taking T cells from patients, but instead of activating and expanding the available anti-tumor T cells, the T cells are equipped with a new (recombinant) TCR that enables them to target specific cancer antigens.

The invention also provides a pharmaceutical composition comprising a T cell as disclosed herein, and a pharmaceutically acceptable excipient.

The term “pharmaceutical composition”, as used herein, includes reference to a composition that is made under conditions such that it is suitable for administration to mammals, preferably humans, e.g., it is made under GMP conditions. A pharmaceutical composition according to the invention may comprise pharmaceutically acceptable excipients, e.g., without limitation, stabilizers, bulking agents, buffers, carriers, diluents, vehicles, solubilizers, and binders. The skilled person understands that the selection of appropriate carriers or diluents depends on the route of administration and the dosage form, as well as the active ingredient and other factors. A pharmaceutical composition according to the invention is preferably adapted for parenteral administration.

The T cells disclosed herein may be administered in the form of any suitable pharmaceutical composition.

The pharmaceutical compositions as referred to are preferably sterile and contain a therapeutically effective amount of a T cell as disclosed herein and a pharmaceutically acceptable excipient such as a carrier or diluent. A pharmaceutical composition may be in the form of an infusable solution or suspension.

The T cell as disclosed herein can be administered through injection or infusion, preferably wherein the T cell as disclosed herein is comprised in a liquid such as an aqeous liquid. Exemplary routes of administration include parenteral administration such as intravenous, intramuscular, intraperitoneal, subcutaneous, intra-arterial and intracerebral administration.

Cell populations comprising T cells according to the present invention can be provided systemically or directly to a subject for the treatment of a neoplasia. In one embodiment, T cells of the present invention are directly injected into an organ of interest (e.g., an organ affected by a neoplasia). Alternatively, T cells and compositions comprising thereof of the present invention are provided indirectly to the organ of interest, for example, by administration into the circulatory system (and providing access to the tumor vasculature). Expansion and differentiation agents and/or immune modulatory agents can be provided prior to, during or after administration of cells and compositions to increase production of T cells in vitro or in vivo.

T cells and pharmaceutical compositions comprising them in accordance with the present invention can be administered in any physiologically acceptable vehicle and to any acceptable site, normally intravascularly, although they may also be introduced into bone or other convenient site where the cells may find an appropriate niche for regeneration and differentiation (e.g., thymus). Usually, at least 1×10⁷ cells will be administered, eventually reaching 1×10¹⁰ or more. A cell population comprising T cells can comprise a purified population of cells. Those skilled in the art can readily determine the percentage of TCR-engineered T cells in a population using various well-known methods, such as flow cytometry. Preferable ranges of purity in populations comprising TCR-engineered T cells are about 5 to about 70%. More preferably the purity is about 20 to about 80%. Dosages can be readily adjusted by those skilled in the art (e.g., a decrease in purity may require an increase in dosage). The cells can be introduced by injection, catheter, or the like. If desired, factors can also be included, including, but not limited to, interleukins, e.g. IL-2, IL-15 and/or IL-21, as well as the other interleukins.

Compositions of the invention include pharmaceutical compositions comprising T cells expressing ROPN1 and/or ROPN1B-specific TCRs and a pharmaceutically acceptable carrier. T cells can be autologous or non-autologous. For example, T cells and compositions comprising thereof can be obtained from one subject, and administered to the same subject or a different, compatible subject. Peripheral blood-derived T cells of the present invention or their progeny (e.g., in vivo, ex vivo or in vitro derived) can be administered via localized injection, including catheter administration, systemic injection, localized injection, intravenous injection, or parenteral administration. When administering a therapeutic composition of the present invention it will generally be formulated in a unit dosage injectable form (solution, suspension, emulsion) and administered intravenously.

Cell populations comprising T cells and compositions comprising T cells in accordance with this invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions comprising T cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard protocols, such as those in “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum Mum monostearate and gelatin. According to the present invention, however, any vehicle, diluent, or additive used would have to be compatible with the T cells of the present invention.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the T cells as described in the present invention. This will present no problem to those skilled in chemical and pharmaceutical principles, or problems can be readily avoided by reference to standard protocols or by simple experiments (not involving undue experimentation), from this disclosure and the documents cited herein.

One consideration concerning the therapeutic use of T cells of the present invention is the quantity of cells necessary to achieve an optimal effect. The quantity of cells to be administered will vary for the subject being treated according to the clinical trial design and protocol. In one embodiment between 10⁷ to 10¹⁰ T cells of the present invention are administered to a human subject. More effective cells may be administered in even smaller numbers. The precise determination of what would be considered an effective dose may be based on factors specific to treatment schedules (i.e., single or combination treatments) and factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, carrier in compositions, and/or co-treatments to be administered in methods of the invention. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %.

For the purpose of clarity and a concise description, features are described herein as part of the same or separate embodiments, however, it will be appreciated that the disclosure includes embodiments having combinations of all or some of the features described.

The content of the documents referred to herein is incorporated by reference.

### NUMBERED EMBODIMENTS THAT ARE ALSO PART OF THE INVENTION

Embodiment 1. An engineered T cell, wherein said T cell is engineered to express a T cell receptor (TCR) that binds to a T cell epitope of human ropporin-1B (ROPN1B), wherein said TCR comprises:
   (i) a T cell receptor alpha chain comprising a hypervariable region that comprises a CDR3 of SEQ ID NO:14, and
   (ii) a T cell receptor beta chain comprising a hypervariable region that comprises a CDR3 of SEQ ID NO:19,
   wherein said hypervariable regions of said T cell receptor alpha and beta chain further comprise a CDR1 and a CDR2.
Embodiment 2. The engineered T cell according to embodiment 1, wherein said hypervariable region of said TCR alpha chain comprises:
   a CDR1 of SEQ ID NO:12;
   a CDR2 of SEQ ID NO:13;
   a CDR3 of SEQ ID NO:14; and/or
wherein said hypervariable region of said T cell receptor beta chain comprises:
   a CDR1 of SEQ ID NO:17;
   a CDR2 of SEQ ID NO:18;
   a CDR3 of SEQ ID NO:19.
Embodiment 3. The engineered T cell according to embodiment 1 or embodiment 2, wherein said T cell receptor alpha chain comprises an amino acid sequence of SEQ ID NO:21 and/or wherein said T cell receptor beta chain comprises an amino acid sequence of SEQ ID NO:22; preferably wherein said T cell receptor alpha chain is that of SEQ ID NO:11 and/or wherein said T cell receptor beta chain is that of SEQ ID NO:16.
Embodiment 4. An engineered T cell, wherein said T cell is engineered to express a T cell receptor (TCR) or a chimeric antigen receptor (CAR) that binds to a T cell epitope of human ropporin-1B (ROPN1B) and/or human ropporin-1A (ROPN1).
Embodiment 5. The engineered T cell according to any one of the preceding embodiments, wherein said T cell epitope is a peptide which forms a complex with a human Major Histocompatibility Complex (MHC) molecule, preferably an HLA-A*02 molecule.
Embodiment 6. The engineered T cell according to any one of the preceding embodiments, wherein said T cell epitope consists of the amino acid sequence selected from one of SEQ ID NOs:1-9 and 20.
Embodiment 7. The engineered T cell according to any one of the preceding embodiments, wherein said T cell epitope consists of the amino acid sequence selected from SEQ ID NO:1 and modified amino acid sequences thereof in which the amino acid residue at position 6 (Glu), position 8 (Glu) and/or position 9 (Val) of SEQ ID NO:1 is substituted by another amino acid residue.
Embodiment 8. A pharmaceutical composition comprising an engineered T cell according to any one of the preceding embodiments, and a pharmaceutically acceptable excipient.
Embodiment 9. A T cell according to any one of embodiments 1-7, wherein said T cell is for use in therapy, preferably for use in the treatment of a solid or liquid (blood) tumor.
Embodiment 10. The T cell for use according to embodiment 9, wherein said solid tumor comprises tumor cells expressing human ROPN1B and/or ROPN1, preferably wherein said solid tumor comprises tumor cells that comprise an MHC molecule that is in complex with, or bound to, a T cell epitope as defined in any one of embodiments 1-7.
Embodiment 11. The T cell for use according to embodiment 9 or embodiment 10, wherein said solid tumor is a breast cancer, preferably a triple negative breast cancer, or a skin cancer, preferably a melanoma.
Embodiment 12. A TCR protein, wherein said TCR protein comprises:
   (i) a T cell receptor alpha chain comprising a hypervariable region that comprises a CDR3 of SEQ ID NO:14, and
   (ii) a T cell receptor beta chain comprising a hypervariable region that comprises a CDR3 of SEQ ID NO:19,
   wherein said hypervariable regions of said T cell receptor alpha and beta chain further comprise a CDR1 and a CDR2.
Embodiment 13. An isolated or purified peptide of human ropporin-1B (ROPN1B) and/or human ropporin-1A (ROPN1), which forms a complex with a human Major Histocompatibility Complex (MHC) molecule, preferably an HLA-A*02 molecule, wherein said peptide consists of the amino acid sequence of any one of SEQ ID NOs:1-9 and 20.
Embodiment 14. An engineered cell, preferably an engineered cancer cell, wherein said cell is engineered to express human ropporin-1B (ROPN1B) and/or human ropporin-1A (ROPN1).
Embodiment 15. A method of treating a subject suffering, or suspected of suffering, from a solid tumor, comprising the step of:—administering a therapeutically effective amount of a T cell according to any one of embodiments 1-7 to a subject in need thereof.

### EXAMPLES

### Example 1. Identifying and Validating Tumor-Restricted Antigen Targets for AT, their Epitopes, their Corresponding TCRs, and Engineered T Cells

Materials and Methods

Generation and Culture of Cell Lines and T Cells

To make a ROPN1-overexpressing triple negative breast cancer (TNBC) cell, a ROPN1B+GFP cDNA fragment (amino acid sequence accessible under UniProtKB Acc. No. Q9BZX4-1) (ROPN1B-2A-GFP) was ordered via GeneArt (Regensburg, Germany) and amplified using PCR with gene-specific primers that included 15 bp extensions homologous to the PiggyBac PB510B-1 vector ends. The amplified fragment was cloned into PiggyBac vector (a kind gift from Dr. P. J. French, Erasmus M C, Rotterdam, the Netherlands) using In Fusion cloning kit (Takara). Subsequently, the MDA-MB-231 cell line (ECACC catalogue no. 92020424, a cell line model for TNBC) was stably transfected with PiggyBac ROPN1B+GFP DNA using Lipofectamine (Invitrogen) and Transposase Expression vector DNA (System Biosciences). The transfected MDA-MB-231 cell line was FACSorted for GFP, after which expression of ROPN1B was confirmed with PCR and immunohistochemical staining of cytospins (using an anti-ROPN1 antibody, see FIG. 3B). Cells of the MDA-MB-231 wildtype and of its ROPN1B-overexpressing variant were cultured in RPMI medium supplemented with 10% FBS, 200 mM L-glutamine and 1% antibiotics without and with 2 μg/mL puromycin, respectively. The packaging cell lines 293T and Phoenix-Ampho were cultured in DMEM supplemented with 10% FBS, 200 mM L-glutamine, nonessential amino acids, and 1% antibiotics (DMEM complete). T2 cells and BSM cells were cultured in RPMI medium supplemented with 10% FBS, 200 mM L-glutamine and 1% antibiotics.

T cells were derived from PBMC from healthy human donors (Sanquin, Amsterdam, the Netherlands) by centrifugation via Ficoll-Isopaque (density=1.077 g/cm3; Amersham Pharmacia Biotech, Uppsala, Sweden), and cultured in RPMI medium supplemented with 25 mM HEPES, 6% human serum (Sanquin, Amsterdam, the Netherlands), 200 mM L-glutamine, and 1% antibiotics (T cell medium) and 360 U/ml human rIL-2 (Proleukin; Chiron, Amsterdam, the Netherlands) and were stimulated every 2 weeks with a mixture of irradiated allogeneic feeder cells, as described elsewhere (Van de Griend et al., Transplantation., 38(4):401-406 (1984)).

Patient Cohorts, Databases and Code of Conduct

TNBC cohort 1: BC with RNAseq (n=347 of which n=66 TNBC, geTMM normalized) accessible through European genome-phenome archive EGAS00001001178 (BASIS cohort).

TNBC cohort 2: Primary BC with node-negative disease with microarray data (U133) who did not receive adjuvant systemic treatment (n=867 of which n=183 TNBC). Data retrieved from gene expression omnibus GSE2034, GSE5327, GSE11121, GSE2990 and GSE7390. Details of combined cohorts have been described previously (Hammerl et al., Clin Cancer Res. 2019. doi:10.1158/1078-0432.CCR-19-0285).

TCGA: Pan-cancer RNAseq data as well as sample annotation data were retrieved from the USCS Xena browser (n=10,495 of which 1,211 BC and 122 TNBC, TPM normalized).

Healthy tissues: RNAseq data of 6 databases covering 66 healthy tissues (Uhlen's Lab: n=122 individuals, n=32 tissues; GTEx: n=1,315 individuals, n=63 tissues; Illumina body map: n=32 individuals, n=17 tissues; human proteome map: n=30 individuals, n=26 tissues; Synders Lab: n=210 individuals, n=32 tissues) were downloaded from Expression atlas (TPM normalized).

This study was performed according to the Declaration of Helsinki and the “Code for Proper Secondary Use of Human Tissue in The Netherlands” (version 2002, update 2011) of the Federation of Medical Scientific Societies (FMSF), the latter aligning with authorized use of coded spare tissue for research. Data analysis and ex vivo analysis was approved by the medical ethical committee at Erasmus MC (MEC.02.953 and MEC-2020-0090, respectively). According to national guidelines, no informed consent was required for this study.

Expression Analysis

Gene Expression (RNAseq, Microarray and qPCR)

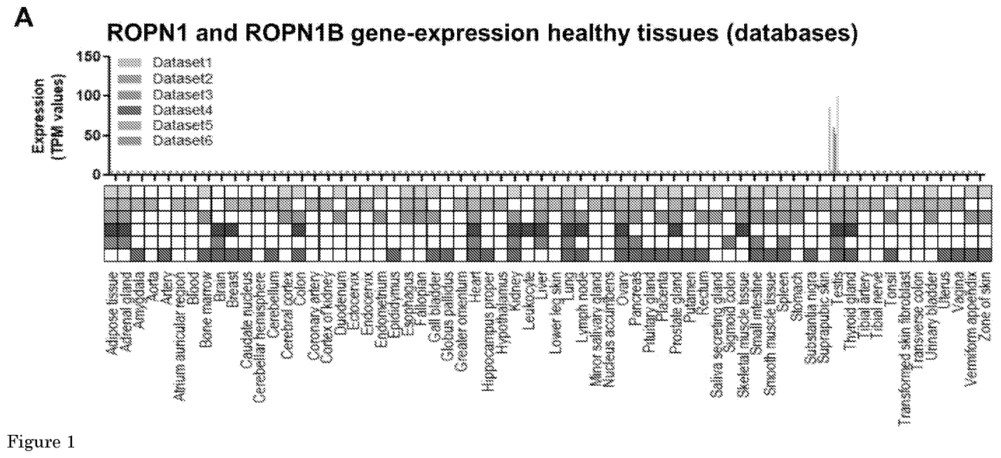

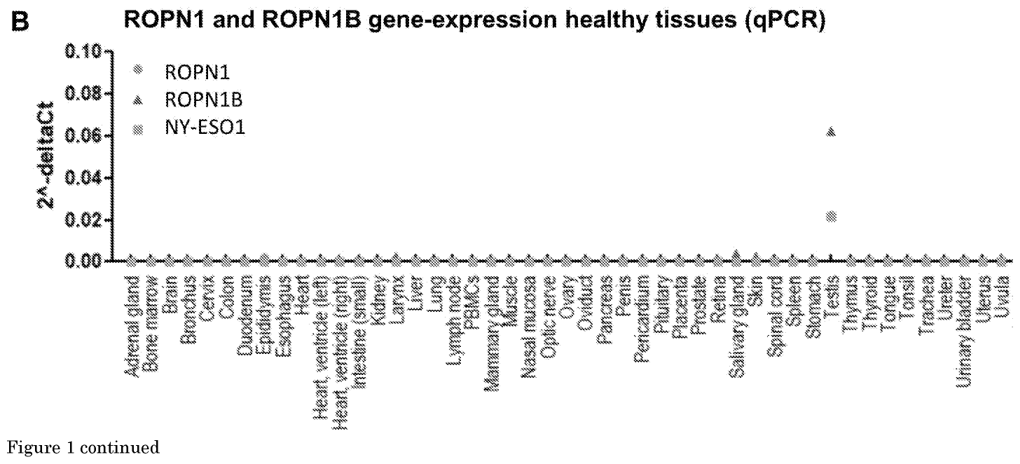

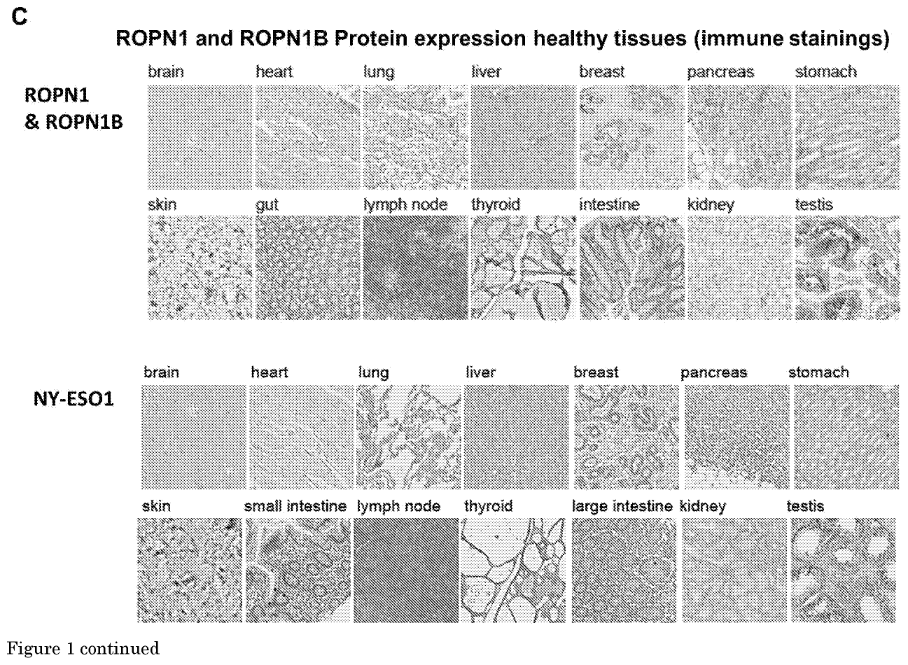

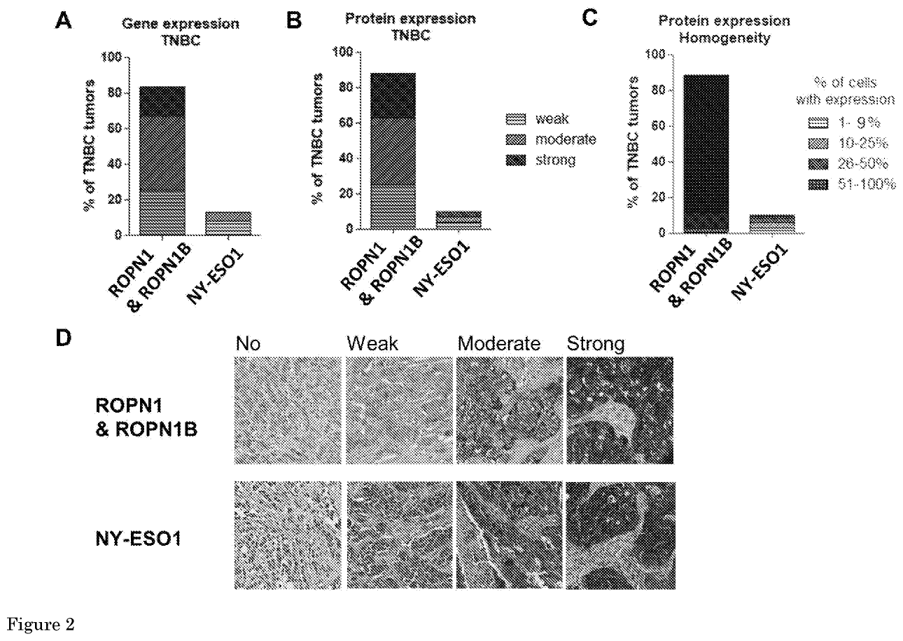

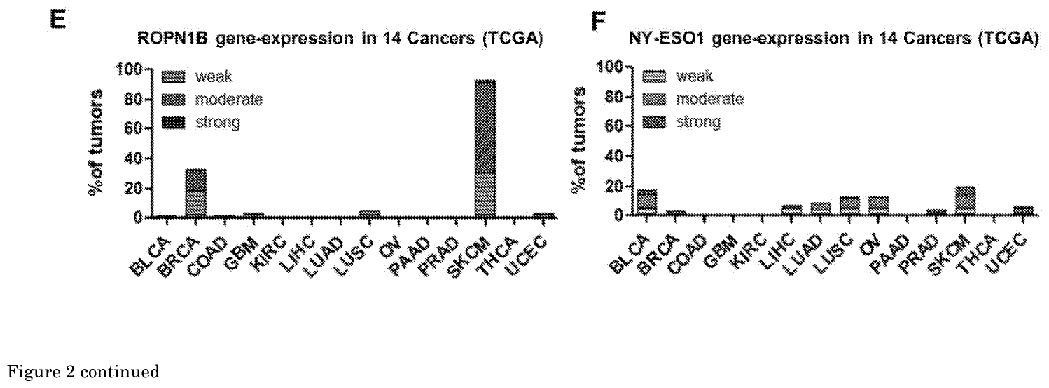

Expression of 239 cancer germline antigens (CGAs, as in Ctdatabase, Ludwig institute, http://www.cta.lncc.br/) was analyzed in healthy and tumor tissues. Expression of ROPN1 and ROPN1B was evaluated in 5 different cohorts of healthy tissues and was considered expressed in a tissue when TPM values reached the threshold of TPM>0.2 in at least 2 cohorts (FIG. 1A). Expression in tumors (TCGA) was classified as follows: TPM values between 1-9, between 10-100, and >100 were valued as low, moderate, and high expression, respectively (FIG. 2A,E). In case of geTMM-normalized RNAseq data (TNBC cohort 1) or microarray data (TNBC Cohort 2), the threshold for expression was set at the third quartile of all CGAs, and CGAs were ranked based on the percentage of positive tumors based on this threshold.

Quantitative PCR (qPCR) was performed on normal human tissue cDNA panels (OriGene Technologies, Rockville, MD) using MX3000 to quantify ROPN1 (TaqMan probe: Hs00250195_m1), ROPN1B (Taqman probe: Hs00250195_m1) and GAPDH (TaqMan probe: Hs02758991_g1) mRNA expression of 48 healthy human tissues (FIG. 1B). Ct values of genes of interest were normalized to the Ct values of GAPDH and relative expression was analyzed by 2^{-dCt}.

Immunohistochemical Staining

IHC stainings were performed using large cores of healthy tissues (2 mm in diameter) covering 16 major tissues from 2-6 individuals (derived from autopsy or resection, n=62) (FIG. 1C) as well as FFPE tissue microarrays of TNBC tumor tissue covering 338 patients which have been described previously (FIG. 2B-D). Staining with anti-ROPN1 antibody (which detects both ROPN and ROPN1B protein) was performed following heat-induced antigen retrieval for 20 min at 95° C. After cooling to RT, staining was visualized by the anti-mouse EnVision+® System-HRP (DAB) (DakoCytomation, Glostrup, Denmark). Human testis tissue was used as positive control tissue. Stainings were manually scored on intensity and percentage of positive tumor cells, using Distiller (SlidePath) software independently by 3 investigators.

Identification, Selection and Ranking of Epitopes

Prediction and Immunopeptidomics

Peptides were selected based on high ranking according to multiple in silico methods to predict different aspects of immune reactivity (Hammerl et al., Trends Immunol. 2018;xx:1-16, doi:10.1016/j.it.2018.09.004) (i.e., NetMHCpan (Hoof et al., Immunogenetics; 61(1):1-13 (2009) doi:10.1007/s00251-008-0341-z); NetCTLpan (Stranzl et al., Immunogenetics; 62(6):357-368 (2010), doi:10.1007/s00251-010-0441-437); SYFPEITHI (Rammensee et al., Immunogenetics; 50(3-4):213-219 (1999), doi:10.1007/s002510050595); and RANKPEP (Reche et al., Hum Immunol; 63(9):701-709 (2002), doi:10.1016/S0198-8859(02)00432-9; Reche et al., Immunogenetics; 56(6):405-419 (2004), doi:10.1007/s00251-004-0709-7; and Reche et al., Methods Mol Bio1; 409:185-200 (2007), doi:10.1007/978-1-60327-118-9_13). For immunopeptidomics, the ROPN1B-overexpressing MDA-MB231 cells (3×10⁸) were treated with IFNy for 24 h and harvested using EDTA before immunoprecipitation of MHC class I molecules. Peptides were eluted and measured with mass spectrometry as described previously. The top 10 predicted peptides per tool (n=17 unique peptides) as well as the unique peptides retrieved from immunopeptidomics (n=2) were checked for cross-reactivity with Expitope (see Table 1) and peptides with up to 2 amino acid mismatches were excluded from further analysis. Selected peptides (n=14; see FIG. 3A) were ordered at ThinkPeptides (Prolmmune, Oxford, United Kingdom), dissolved in 50-75% DMSO and stored at −20° C. until use.

HLA-A2 stabilization assay and ranking of epitopes. The HLA-A2 stabilization assay was performed using T2 cells as described in Miles et al., Mol Immunol; 48(4):728-732 (2011), doi:10.1016/j.molimm.2010.11.004. In short, 0,15×10⁶ T2 cells (the LCLxT lymphoblastoid hybrid cell line 0.1743CEM.T2) were incubated with titrated amounts of peptide for 3 h at 37° C. in serum-free medium supplemented with 3 μg/mL @2-microglobulin (Sigma). Surface expressed HLA-A2 molecules were measured with flow cytometry using the HLA-A2 mAb BB7.2 (BD Pharmingen, 1:20). To this end, T2 cells were washed, and stained using fluorescently-labeled antibody, incubated for 25 min on ice in the dark, and dissolved in PBA with 1% FBS. Cells were gated for viability using flow cytometry, and events were acquired on a FACSCanto flow cytometer and analyzed using FlowJo software (TreeStar, Ashland, OR). T2 cells without peptide were used as baseline. In a first screen, peptides used at a concentration of 25 ug/ml that induced >1.1-fold change over baseline (11 out of 14, see FIG. 3C) were further titrated from 0,316 to 31,6 μg/ml. From dose titration curves (FIG. 3D) we have calculated two parameters of binding avidity to HLA-A2: (1) amplitude, which was the difference in fluorescence intensity between the highest concentration and baseline; and (2) half-maximal effective concentration (EC50), which was calculated using GraphPad software. Thresholds for these 2 parameters were: half of the amplitude of the gp100 YLE control peptide; and EC50<1E-5 M. The remaining ROPN1 and ROPN1B T cell epitopes (n=9) were subsequently ranked based on EC50 values (FIG. 3E).

Enrichment of T Cells

Enrichment of ROPN1 Epitope-Specific CD8 T Cells

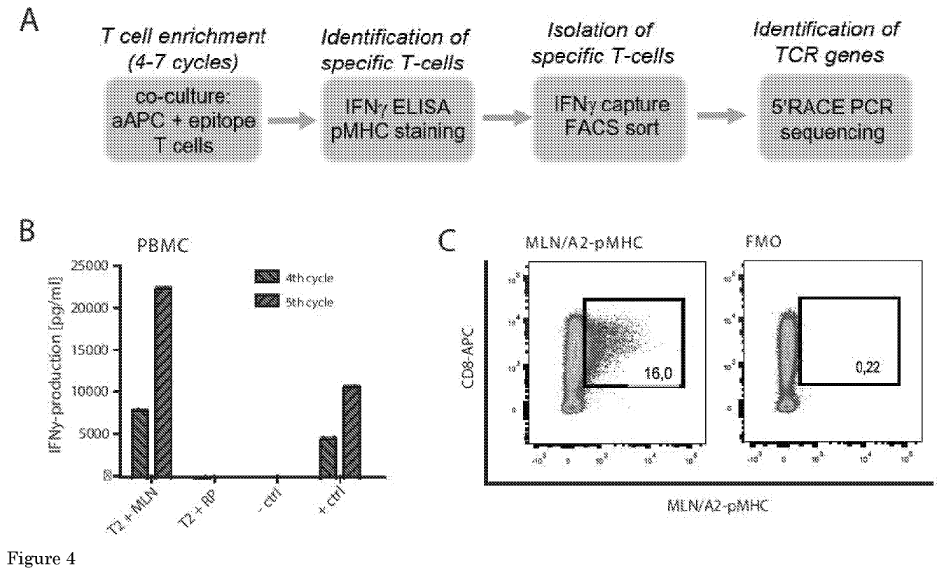

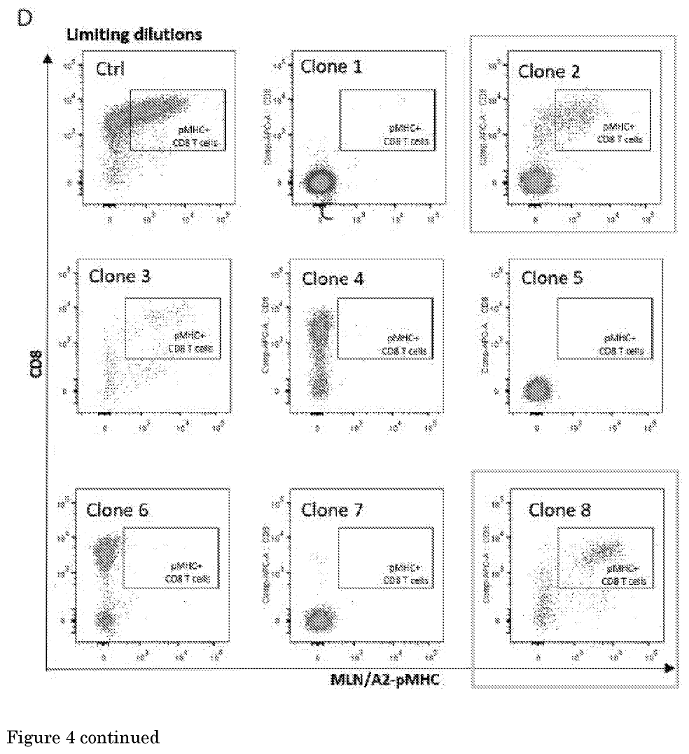

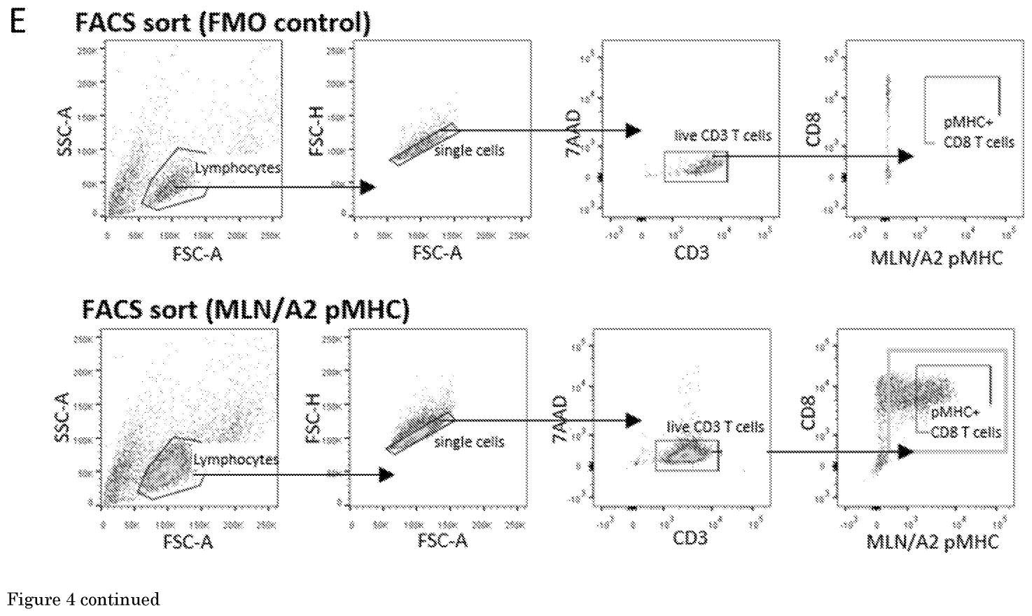

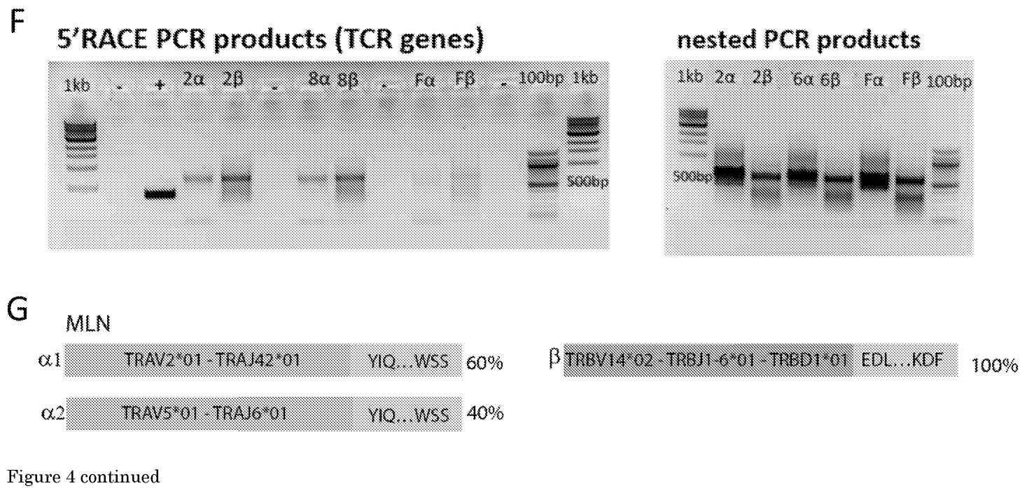

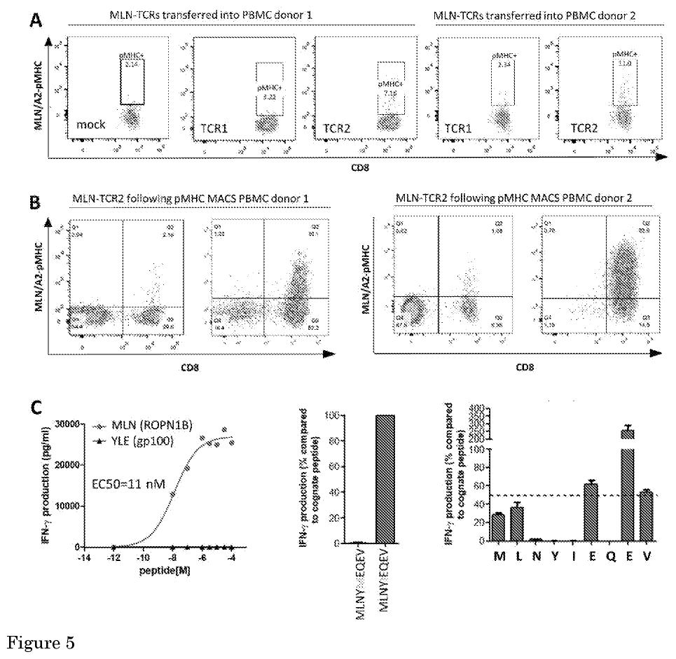

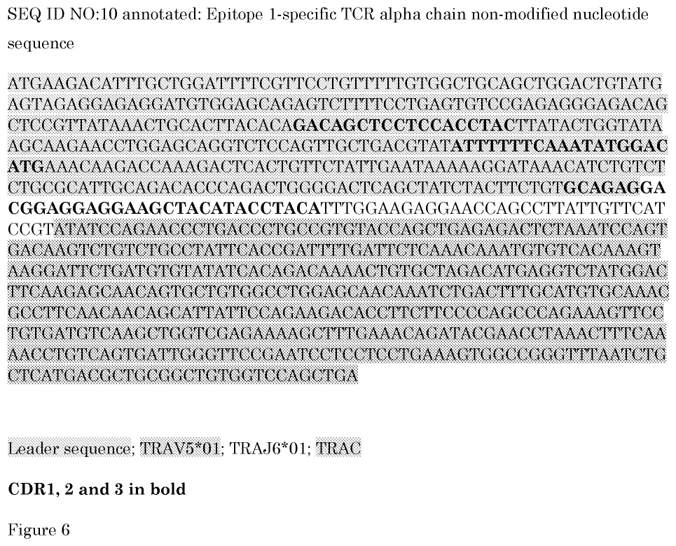

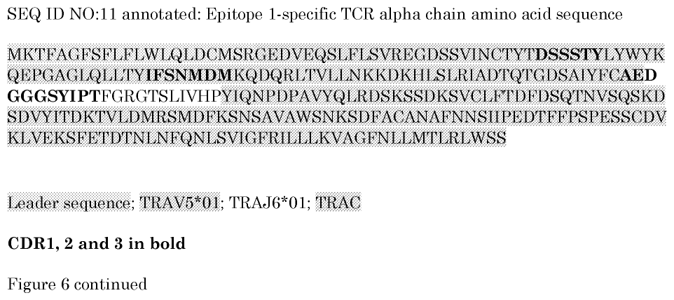

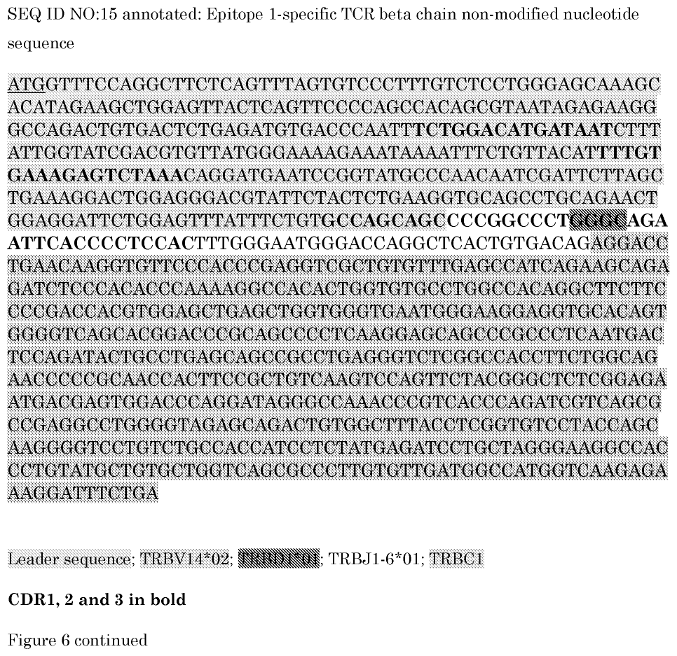

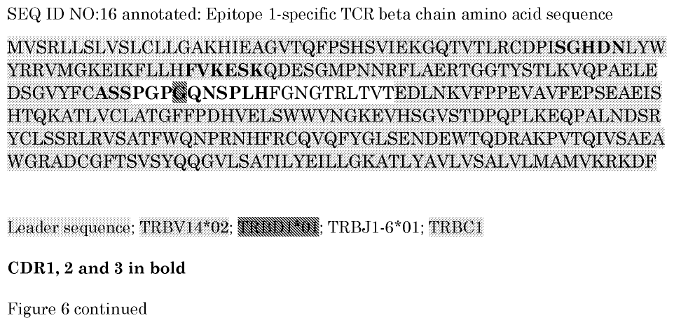

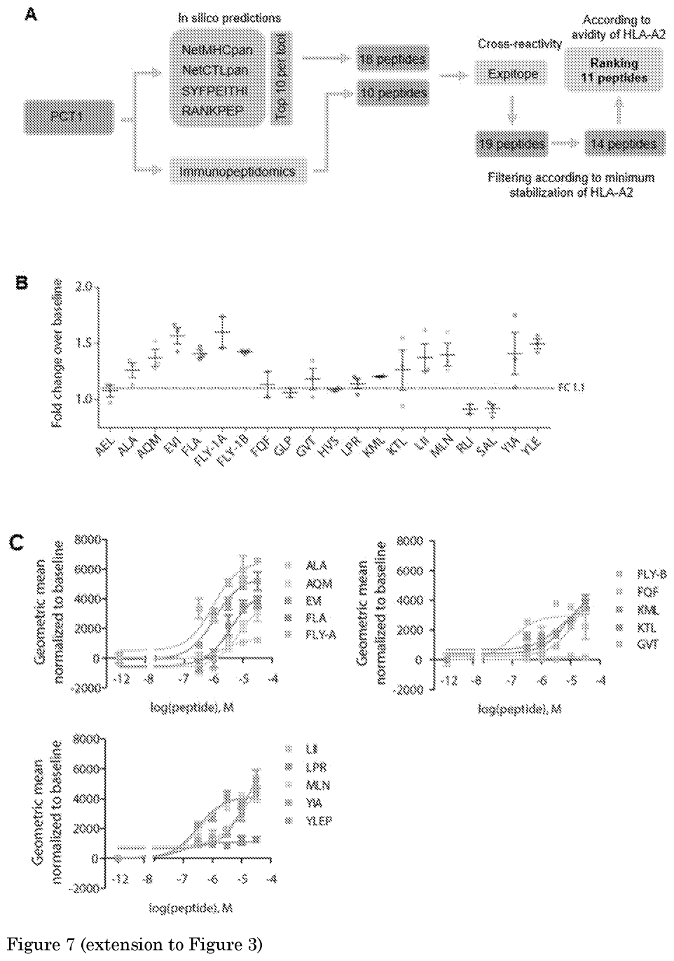

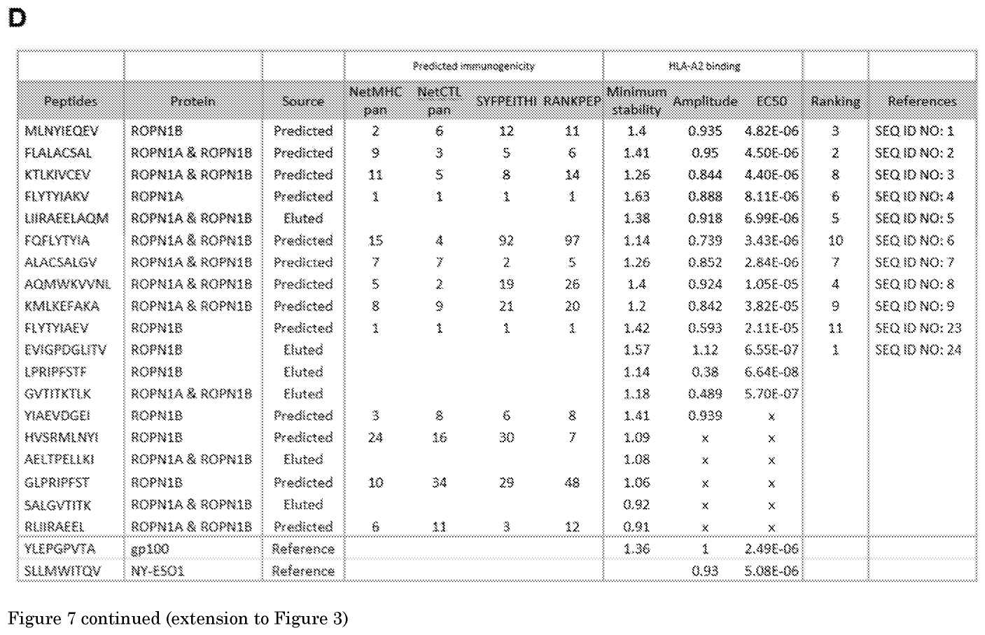

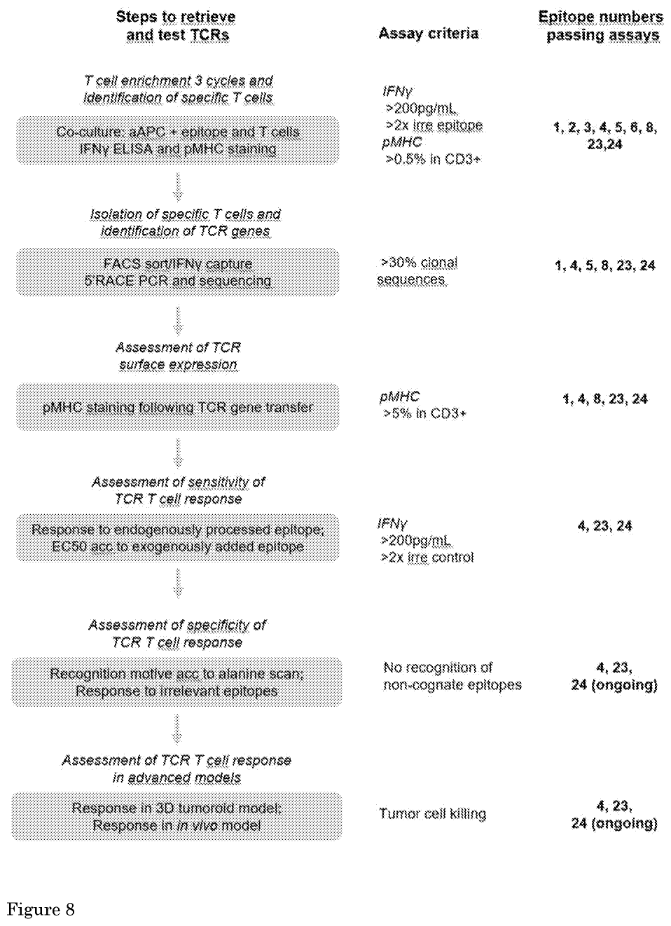

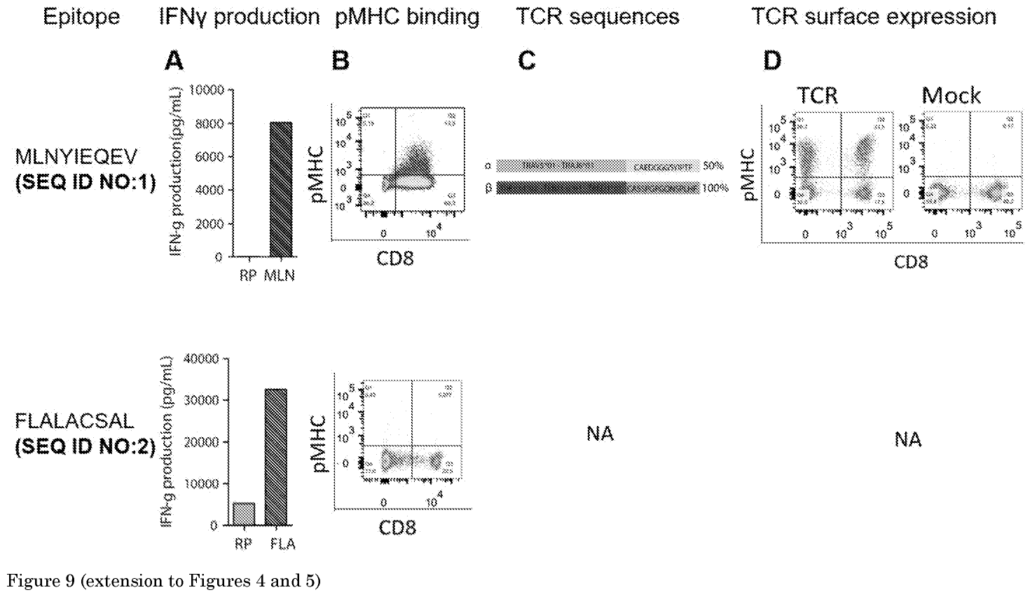

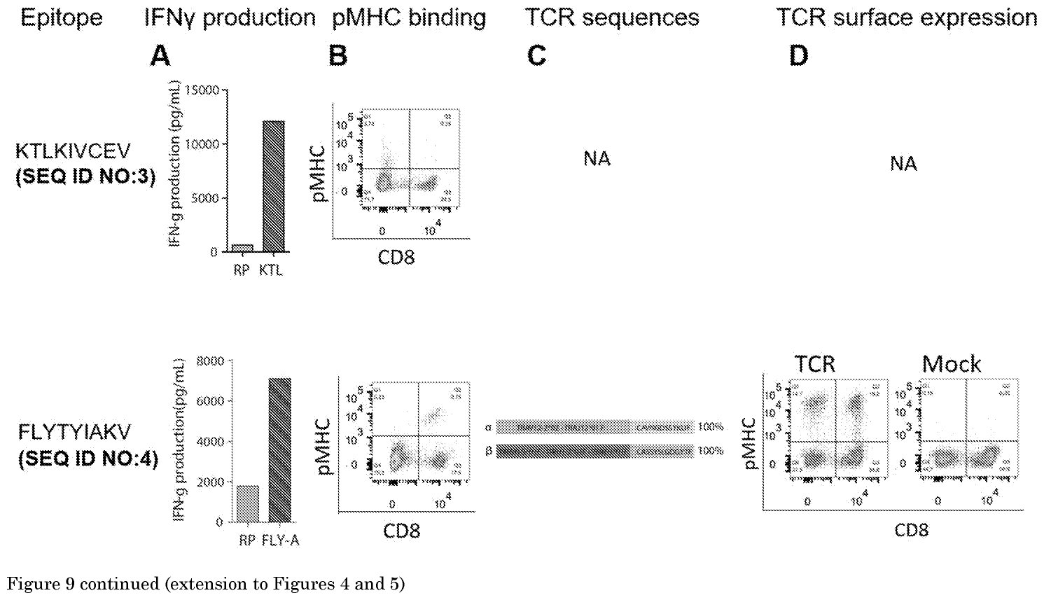

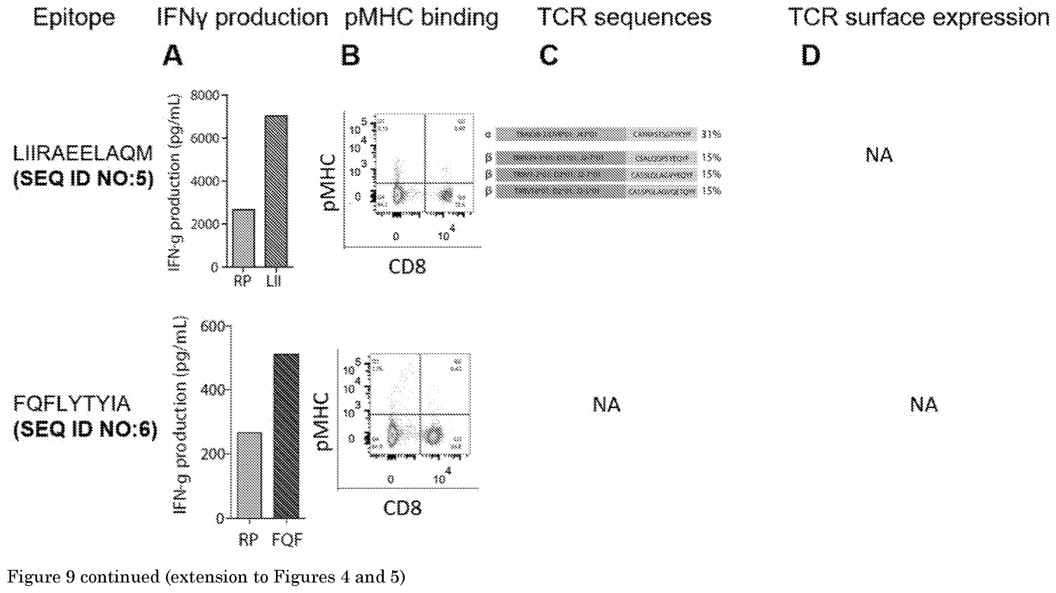

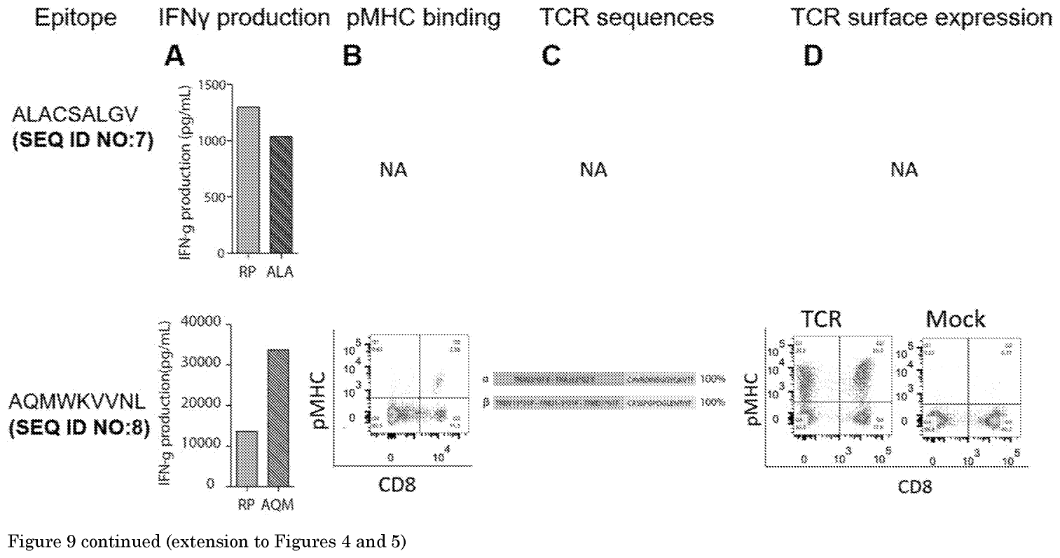

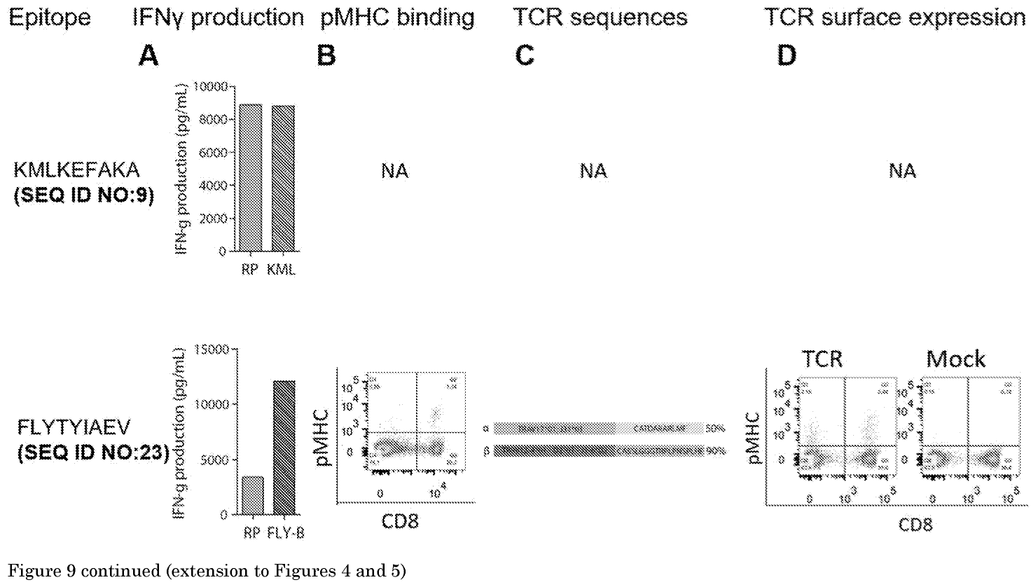

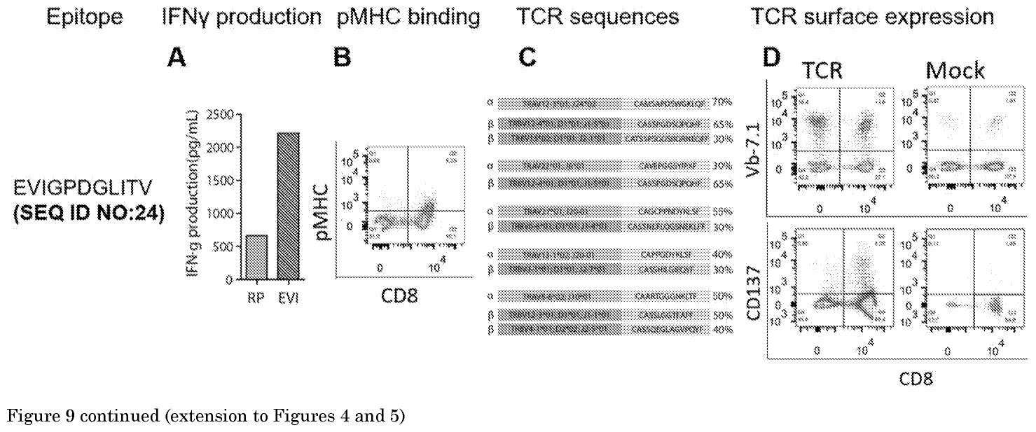

Enrichment of epitope-specific CD8 T cells was performed according to the protocol described by Butler et al., Clin Cancer Res.;13(6):1857-1867 (2007), doi:10.1158/1078-0432, but included the following amendments. CD8+ T lymphocytes were collected from PBMCs by magnetic-activated cell sorting (MACS) according to the CD8 isolation kit (Miltenyi Biotec). CD8+ T cells with >95% purity were subsequently cultured in T cell medium supplemented with IL-2 (36 IU/mL; but no gentamycin) for 1 day, after which expansion cycles started. K562ABC cells (kindly provided by prof. Bruce Levine, University of Pennsylvania, PA) were loaded with 10 μg/ml ROPN1 and/or ROPN1B peptide and incubated for 5 h at RT in serum-free medium, after which cells were washed and fixed with 0,1% paraformaldehyde. After washing, K562ABC cells were suspended at 0.1×10⁶/mL and co-cultured with T cells in a 1:20 ratio. IL-2 (36 IU/mL) and IL-15 (20 ng/μL) were added at days 1 and 3 days after start of co-culture, and after 6 days, T cells were counted, suspended at 2×10⁶/mL and rested for 1 day, after which the next cycle commenced (this schedule continued up to 4 or 5 cycles). Following 4 and 5 cycles, T cells were stained with pMHC multimers (HLA*0201/MLN, Immudex, Copenhagen, Denmark). pMHC-PE were pre-incubated at RT for 10 min followed by incubation with 7AAD, anti-CD3-FITC and anti-CD8-APC for 20 min. Cells were fixed with 1% paraformaldehyde and TCR expression was measured with flow cytometry and analyzed using FlowJoX software. FIG. 4A schematically depicts the procedure to enrich epitope-specific T cells, and obtainthe corresponding TCR genes.

Enriched T cells were tested for ROPN1 and/or ROPN1B epitope-specific IFN_{Y} production. To this end, T2 cells (4×10⁶/mL) were loaded with peptide (20 ng/mL) for 30 min. T cells (2×10⁵) were cultured in a 1:1 ratio with T2 cells in a round bottom 96-wells plate, and the next day supernatant was collected and IFN-γ production was measured with an Enzyme-linked immunosorbent assay (ELISA, Invitrogen) according to manufacturer's protocol (FIG. 4B). T2 cells without peptide were included as a negative control; and staphylococcal enterotoxin B (0.1 μg, Sigma) was used as a positive control.

To obtain ROPN1 and/or ROPN1B epitope-specific TCRs, enriched T cells (FIG. 4C) were either single cell diluted following IFNy secretion (Milentyi Biotec) or FACS-sorted with pMHC multimers (see FIG. 4D,E for examples). For the former procedure, T cells were stimulated with irradiated BSM cells, and IFN_{Y}—secreting cells were captured according to the manufacturer's recommendations.

TCR Cloning and Sequence Identification

CD8 T cells from either procedure, were exposed to the SMARTer™ RACE cDNA Amplification Kit (Clontech) to identify ROPN1 and/or ROPN1B epitope-specific TCRα- and β-chains. In brief, RNA was isolated by spin column purification (NucleoSpin, Macherey-Nagel) after which 5′RACE ready cDNA was made as in Kunert et al., J Immunol; 197(6):2541-2552 (2016), doi:10.4049/jimmunol.1502024, and PCR was performed to amplify TCR-V encoding regions (FIG. 4F). Initial products were re-amplified by nested PCRs, cloned into the TOPO 2.1 vector (Invitrogen), and subjected to DNA sequencing. TCRα and TCRβ sequences were verified in at least twelve colonies. Using the IMGT database and the HighV-QUEST tool (http://www.imgt.org), the TCR V, D, and J sequences were annotated according to the Lefranc nomenclature (see FIG. 4G).

TCR Gene Transfer and In Vitro Testing

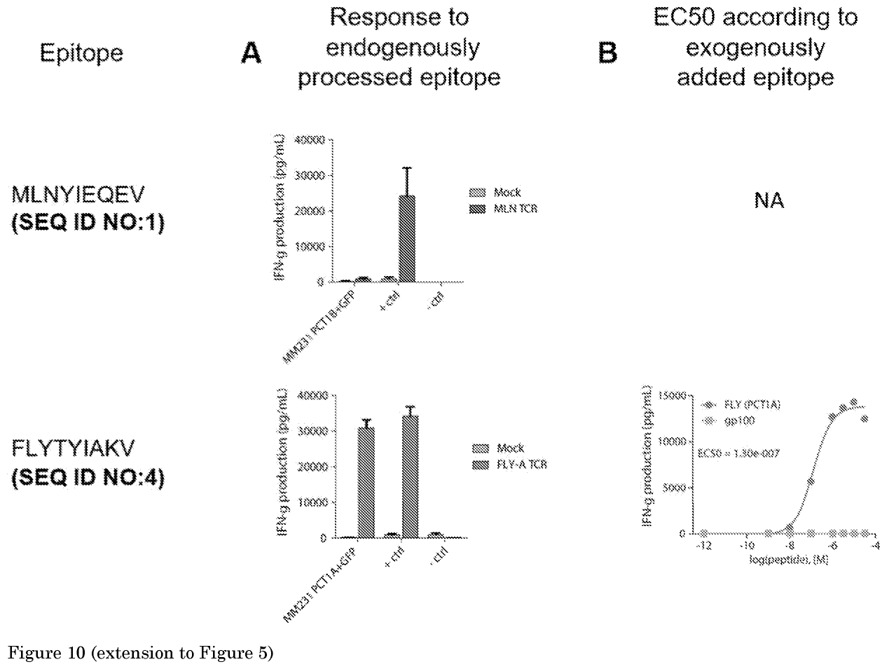

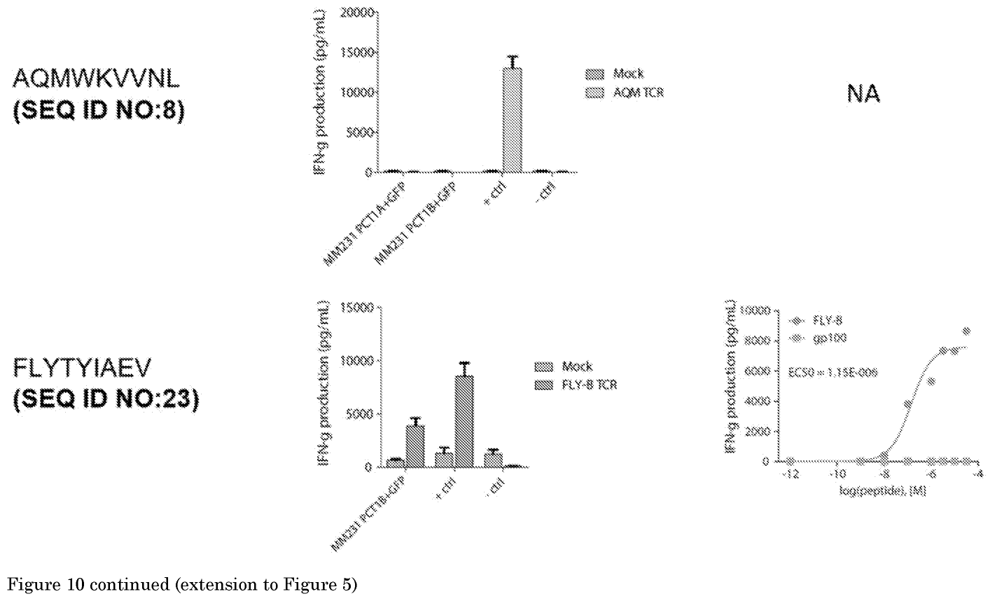

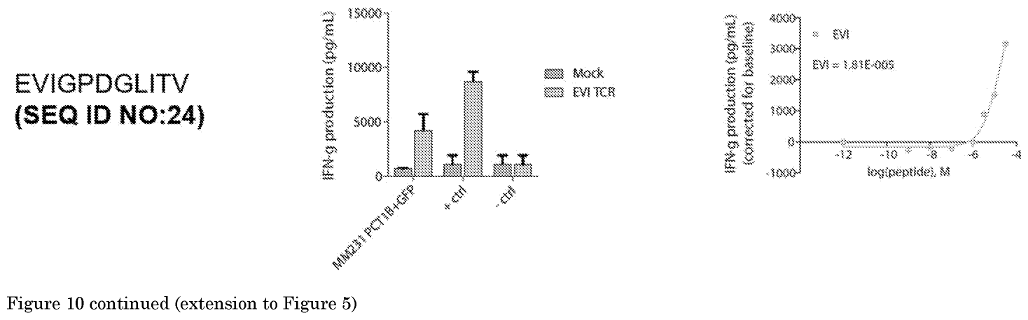

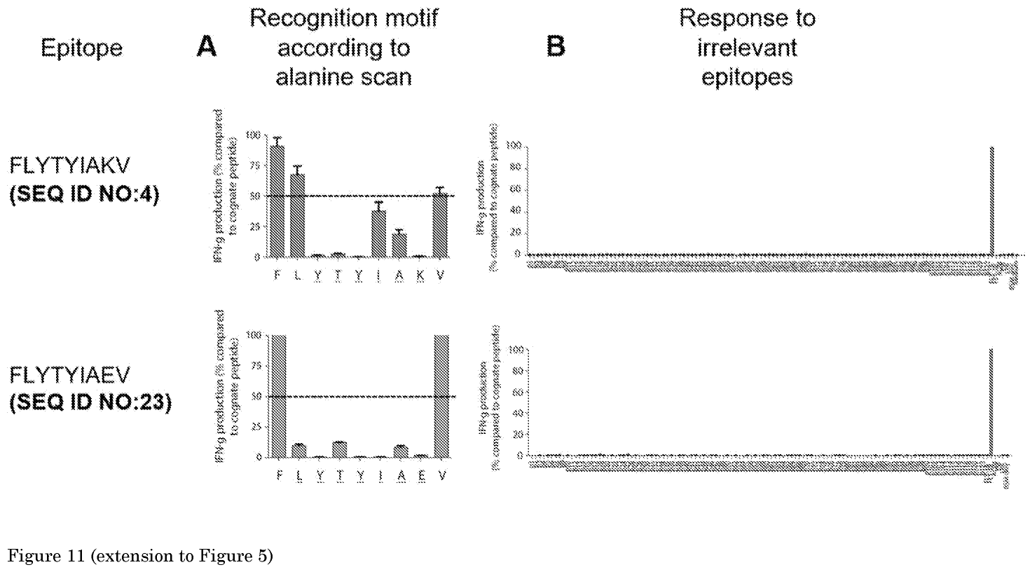

Identified TCRα and TCRβ genes were codon optimized (GeneArt, Regensburg, Germany) and cloned into the pMP71 vector (a kind gift of prof. Wolfgang Uckert, MDC, Berlin, Germany) using a TCRβ-2A-TCRα cassette that was flanked by NotI and EcoRI restriction sites. Upon activation with anti-CD3 mAb OKT3, PBMCs from healthy donors were transduced with TCR-encoding retroviruses (pMP71) or empty vector that were produced by a co-culture of 293T and Phoenix-Ampho packaging cells, as described previously in Lamers et al., Cancer Gene Ther.;13(5):503-509 (2006), doi:10.1038/sj.cgt.7700916, and Straetemans G, Clin Dev Immunol, 2012. Staining for surface-expressed TCR transgenes was performed as described above (FIG. 5A,B).

Transduced T cells (6×10⁴/well in a 96-well plate) were co-cultured with BSM cells (loaded with peptide concentrations ranging from 1 pM to 1 μM) or tumor cells (2×10⁴/well) in a total volume of 200 μl of T cell medium for 24 h at 37° C. Responses and EC50 of ROPN1, ROPN1B and gp100 control peptide required for T cell IFN-γ production were calculated using GraphPad Prism 5 software (FIG. 5C).

The recognition motive was determined in a co-culture of T2 cells loaded with peptides containing individual alanines as replacements at every single position in the cognate ROPN1B peptide. Critical positions were determined as >50% decrease in IFNy production, compared to the cognate peptide. The resulting motive was scanned for occurrence in the humane proteome using the ScanProsite tool (https://prosite.expasy.org/scanprosite/) (FIG. 5C, and SEQ ID NO:20).

Results

ROPN1 and ROPN1B are Absent from Healthy Tissues and Show Abundant and Homogenous Expression in >80% of TNBC

To identify TNBC-specific target antigens for adoptive T cell therapy (AT), gene-expression values were interrogated of all currently known CGAs (n=239); in 5 databases covering 66 healthy tissues derived from a total of 1,735 individuals, as well as in large databases of 447 TNBC patients and 6,670 cancer patients covering 14 solid tumor types. These gene expressions (or their absence) were subsequently validated by qPCR and immunohistochemistry (IHC). Using this work flow (see M&M for details), ROPN1 and ROPN1B were identified as a target for AT to treat TNBC according to the following outcomes: First, ROPN1 or its isoform ROPN1B were not expressed in any healthy tissue, except for testis (according to gene expression databases, FIG. 1A). Gene expression level of the reference CGA NY-ESO1 (CTAG1B) in healthy tissues (except immune-privileged sites, such as testis, placenta and epididymis) was set as an expression threshold (TPM≤0.2) since this antigen has been successfully targeted with TCR-engineered T cells without treatment-related toxicities (Rapoport et al., Nat Med. 21(8):914-921 (2015), doi:10.1038/nm.3910; and Robbins et al., Clin Cancer Res. 2015, doi:10.1158/1078-0432.CCR-14-2708). Absent expression in major healthy tissues of both ROPN1 isoforms and NY-ESO1 was confirmed by qPCR using commercially obtained cDNA libraries of 48 healthy tissues pooled from 10 donors (FIG. 1B) as well as IHC stainings of tissue microarrays containing 16 major healthy tissues from 2-6 individuals (FIG. 1C). Second, ROPN1 and ROPN1B were highly expressed by 84% of TNBC patients (FIG. 2A) as well as a 93% of skin-cutaneous melanoma and to a much lesser extent in a number of other solid tumor types (FIG. 2E: UCEC: 3%, LUSC: 5%, GBM:3%). High ROPN1 and ROPN1B gene-expressions in TNBC were confirmed in two additional gene-expression datasets (TNBC Cohort 1: 86% positive, n=66 patients; and TNBC Cohort 2: 77% positive, n=259 patients). In comparison NY-ESO1 was expressed by ≤14% of TNBC patients and expression-levels were generally low (FIG. 2E). In addition to gene expression, ROPN1 and ROPN1B protein expressions were demonstrated in 88% of TNBC via immunohistochemical (IHC) staining of tumor tissue microarrays (n=338) (FIG. 2B). Third, protein expression was homogenous in 83%, whereas heterogeneous expression (<50% of tumor cells being positive for ROPN1 and ROPN1B) was observed in only 13% of ROPN1 and ROPN1B-positive TNBC, while NY-ESO1 was homogenously expressed in 18% and heterogeneously expressed in 82% of NY-ES01-positive TNBC (FIG. 2C,D).

### Predicted and Eluted ROPN1 and ROPN1B Epitopes are Avidly Bound by HLA-A2

To select immunogenic T cell epitopes, we first performed a series of in silico predictions using the following tools: NetMHC, NetCTLpan, RANKPEP, and SYFPEITHI, which are each biased towards various qualitative aspects of epitopes, such as presence of cleavage sites, affinity for transport of associated proteins (TAP), and affinity for binding to HLA (see FIG. 3A for an overview of epitope characterization and selection, see for details on predicted features Hammerl et al., Trends Immunol. 2018;xx:1-16. doi:10.1016/j.it.2018.09.004). Predicted epitopes were ranked per tool and the top 10 peptides of each tool were weighted and ranked, resulting in 17 unique peptides. Secondly, we used MDA-MB-231 cells (a TNBC cell line with high HLA-A2 expression) that over-expressed ROPN1B as a source for immunopeptidomics. We have validated ROPN1B-GFP expression by immune staining and flow cytometry (FIG. 3B). Mass-spectrometry analysis of all MHC class I-bound peptides yielded 2 additional unique epitopes (FIG. 3B). The total set of unique epitopes (n=19) were screened for non-homology to other peptide sequences present in the human proteome using the algorithm EXPITOPE, which yielded 14 immunogenic, non-cross-reactive ROPN1 and/or ROPN1B peptides (i.e., peptides with >2 mismatches compared to any other peptide, Table 1). These 14 peptides along with the reference and control peptides NY-ES01 (SLLMWITQV) and gp100 (YLEPGPVTA) were tested for binding to HLA-A2 in vitro. In a first side-by-side screen using a saturating concentration (25 μg/ml), peptides were selected that induced >1.1-fold change over baseline (considered a minimal stability of HLA-A2, FIG. 3C). Three predicted peptides did not reach this threshold. Interestingly, AELTPELLKI (10-mer, derived from immunopeptidome) did not bind to HLA-A2 (and was mapped in silico to HLA-B40:01), and LIIRAEELAQM (11-mer also derived from immunopeptidome) did bind to HLA-A2 with high affinity (comparable to the top predicted HLA-A2 binders). Notably, the latter peptide was not predicted to bind to HLA-A2 binding. The 11 remaining peptides were analyzed by dose titrations (FIG. 3D), and excluded when displaying maximum binding that was half or less when compared to the gp100 peptide (i.e., amplitude) or when displaying an EC50 less than 1E⁻⁵ M. Nine peptides survived these criteria, and were ranked according to EC50 values (FIG. 3E; and SEQ ID NOs:1-9).

ROPN1B Epitope-Specific CD8 T Cells are Enriched from Healthy Donor T Cells

We used the 5 top-ranked epitopes (FQFLYTYIA, EC50: 3.301; KTLKIVCEV, EC50: 4.401; FLALACSAL, EC50: 5.1 μM; MLNYIEQEV, EC50: 6.6 μM; FLYTYIAEV, EC50: 1.1 mM), all having similar EC50 values when compared to the NY-ESO1 peptide (SLLMWITQV, EC50: 501), in co-cultures of T cells and artificial antigen presenting cells (aAPC, K562ABC overexpressing HLA-A2, CD80 and CD86) (according to Butler et al., Sci Transl Med.3(80):80ra34-80ra34 (2011), doi:10.1126/scitranslmed.3002207). T cells were either isolated healthy donors, and following 4-5 enrichment cycles tested for epitope-specific IFN_{Y} production (see for an overview of the T cell enrichment procedure see FIG. 4A). Two HLA-A2-positive donors were tested and we enriched up to now MLN-epitope-specific T cells in 1 healthy donor (FIG. 4B). Enriched T cells harbored 20% and 62% binding MLN/HLA-A2 complexes after 4 and 5 cycles, respectively (FIG. 4C). These T cells were either cloned through limiting dilutions following IFN_{Y} capture (FIG. 4D) or sorted through FACS using corresponding pMHC multimers (FIG. 4E), and subsequently used to identify epitope-specific TCR genes via 5′RACE PCR (FIG. 4F). MLN-TCR genes contained 2 genes encoding for the variable TCRα chain (TRVA) and 1 gene encoding for the variable TCRβ chain (TRVB) (FIG. 4G).

### MLN Epitope-Specific TCR is Functionally Expressed by T Cells

The MLN TCR-αβ combinations were codon-optimized, and cloned into pMP71. Testing for surface expression in peripheral T cells from 2 healthy donors demonstrated that MLN TCR2 (FIG. 5A,B) resulted in binding of MLN peptide:HLA-A2. In subsequent experiments, MLN TCR2 T cells were MACS-sorted using pMHC complexes, and this TCR was shown to mediate recognition of the ROPN1B epitope but not an irrelevant epitope with an affinity of 11 nM (FIG. 5C). Furthermore, this TCR specifically recognized the MLN epitope derived from ROPN1B (MLNYIEQEV) but not ROPN1 (MLNYMEQEV) with only a single amino acid difference (FIG. 5C) and showed a stringent recognition motif as determined by alanine scan (see Materials and Methodssection for details). Every amino acid, except for the glutamic acids at positions 6 and 8 and the valine at position 9, were crucial for TCR recognition (FIG. 5C), and the resulting recognition motif: M-L-N-Y-I-x-Q-x-x was not present in any other known sequence of the human proteome.

### DISCUSSION

In the current study, we utilized a workflow of in silico and laboratory tools to identify a tumor-selective and immunogenic target antigen, corresponding T cell epitopes and TCRs for the treatment of TNBC. Importantly, with this workflow we aimed to address three challenges in the field of AT, namely: T cell-related toxicities; heterogeneous expression of target antigens; and selection of suboptimal T cell epitopes.

The most prominent challenge of AT with engineered T cells is the risk for T cell-related toxicities, i.e., on- and off target toxicities. We utilized an approach that was aimed to minimize these toxicity risks. First, we have selected a target antigen with tumor-restricted expression. In other words, ROPN1 and ROPN1B were screened for absent expression in healthy tissues except for testis and epididymis where ROPN1 and ROPN1B are normally expressed in the fibrous sheath of the sperm. These latter tissues are immune-privileged and not present in women, further minimizing the risk for on-target toxicities in female TNBC patients. Second, ROPN1 and ROPN1B epitopes were screened for non-homology to other peptide/protein sequences in the human proteome (using the algorithm EXPITOPE). Third, TCRs were screened for epitope specificity and absence of cross-reactivity to similar epitopes with a series of in vitro assays. The other challenge of AT is the generally low and heterogeneous expression of target antigens, which is considered to significantly contribute to lack of sustained responses or relapse due to the outgrowth of antigen-negative tumor cell clones. ROPN1 and ROPN1B showed not only tumor-selective, but also high expression in >80% of TNBC, of which the majority had a strict homogeneous expression, indicating that ROPN1 and ROPN1B are expected to represent not only safe, but also effective targets for AT. The third challenge is the selection of truly immunogenic epitopes, to which end we made use of multiple in silico and laboratory tools. Our data revealed little concordance among different techniques. For example, we have identified a naturally occurring HLA-A2-binding epitope (LIIRAEELAQM) through immunopeptidome analysis that was not predicted to bind to HLA-A2. Vice versa, we observed T cell responses in healthy donors against a predicted 9-mer (MLNYIEQEV) that was not retrieved by immunopeptidome analysis. These observations underline the relevance of multiple tools to accurately identify immunogenic epitopes. Furthermore, we argue that validation of HLA-binding in vitro is a prerequisite to exclude falsly predicted epitopes and ensure immunogenicity. In fact, high binding affinity of epitopes towards HLA-A2 enhances cross-presentation through antigen-presenting cells, which has proven important for effective anti-tumor T cell responses (Engels et al., Cancer Cell. 23(4):516-526 (2013), doi:10.1016/j.ccr.2013.03.018; and Kammertoens et al., Cancer Cell. 23(4):429-431 (2013), doi:10.1016/j.ccr.2013.04.004). Collectively, our data suggest that sequential use of multiple prediction tools, in combination with immunopeptidomics, is needed to identify unique and immunogenic epitopes.

Once target antigens and epitopes have been selected, the next step comprises enrichment of epitope-specific T cells and their TCRs. Obtaining epitope-specific TCRs from healthy donor PBMC is generally challenged by very low frequencies of such T cells. In line, we were able to enrich for ROPN1B-specific T cells in 1 out of 2 donors, requiring several enrichment cycles in order to identify the corresponding TCR genes. The identification of identical TCR genes using different approaches suggests that the antigen specific responses originated from a single T cell. Thus high numbers of PBMC are needed to enrich for tumor antigen-specific T cells from healthy donors, which based on our results represent a viable source of such T cells.

To date no studies have been conducted using AT with TCR-engineered cells in TNBC. Chimeric Antigen Receptor (CAR) T cells directed against the tyrosine kinase-like orphan receptor 1 (ROR1) to treat TNBC are currently in clinical trials (Specht et al., Cancer Res. 79(4 Supplement):P2-09-13 LP-P2-09-13 (2019), doi:10.1158/1538-7445.SABCS18-P2-09-13). Preclinical studies have shown that ROR1 CARs can recognize and kill TNBC cells, which frequently overexpress ROR1. Nevertheless, ROR1 is expressed in a variety of healthy tissues, and we argue that ROR1 as a T cell target presents with increased risk for on-target toxicities.

### CONCLUSION

Established and utilized herein is an effective workflow to identify and validate tumor-restricted antigen targets for AT, their epitopes, their corresponding TCRs, and engineered T cells. Amongst other, we identified ROPN1 and ROPN1B as tumor targets for AT with absent expression in multiple healthy tissues, which implies minimal risk for on-target toxicity. Also, we isolated a ROPN1B-specific and HLA-A2-restricted TCR that was expressed in peripheral T cells from healthy donors and that mediates recognition of the MLNYIEQEV epitope but not an irrelevant epitope and showed a stringent recognition motif that is not present in any other human protein which imply minimal risk for off-target toxicities. With these results it is demonstrated that ROPN1B, and expectedly ROPN1, represents an excellent target antigen and ROPN1B-TCRs provide a novel treatment opportunity against cancers that display T cell epitopes of ROPN1B, which is the case for >80% of TNBC patients.

TABLE 1
Overview of identified ROPN1 and ROPNIB epitopes,
and their non-cross reactivity (in bold)

| | | 0 | 1 | 2 |
|---|---|---|---|---|
| Soure | Peptides | mismatch | mismatch | mismatch |
| Predicted | FLYTYIAKV | No | No | No |
| | MLNYIEQEV | No | No | No |
| | YIAEVDGEI | No | No | No |
| | DLFNSVMNV | No | No | 5 proteins |
| | AQMWKVVNL | No | No | No |
| | RLIIRAEEL | No | No | No |
| | ALACSALGV | No | No | No |
| | KMLKEFAKA | No | No | No |
| | FLALACSAL | No | No | No |
| | GLPRIPFST | No | No | No |
| | KTLKIVCEV | No | No | No |
| | ELTPELLKI | No | No | 3 proteins |
| | FQFLYTYIA | No | No | No |
| | LLKILHSQV | No | No | 1 protein |
| | HVSRMLNYI | No | No | No |
| | LTPELLKIL | No | No | >5 proteins |
| | TITKTLKIV | No | No | 2 proteins |
| Eluted | AELTPELLKI | No | No | No |
| | LIIRAEELAQM | No | No | No |

TABLE 2
Overview of identified ROPN1 and ROPNIB epitopes,
their binding to HLA-A2 and immunogenicity

| | | | | | T cell |
|---|---|---|---|---|---|
| | | | | Affinity | responses |
| | | | | for | observed |
| | | | HLA- | HLA | (IFNg) against |
| Protein | epitopes | source | subtype | A2:01 (M) | epitope |
| ROPN1B | MLNYIEQEV | predicted | HLA-A2: 01 | 6.56E−06 | 1/3 co-cultures |
| | (SEQ ID NO: 1) | | | | |
| ROPN1 and | FLALACSAL | predicted | HLA-A2: 01 | 5.05E−06 | 2/5 co-cultures |
| ROPN1B | (SEQ ID NO: 2) | | | | |
| ROPN1 and | KTLKIVCEV | predicted | HLA-A2: 01 | 4.35E−06 | 2/4 co-cultures |
| ROPN1B | (SEQ ID NO: 3) | | | | |
| ROPN1 | FLYTYIAKV | predicted | HLA-A2: 01 | 1.09E−05 | 1/4 co-cultures |
| | (SEQ ID NO: 4) | | | | |
| ROPN1 and | LIIRAEELAQM | eluted | HLA-A2: 01 | 6.99E−06 | Not determined |
| ROPN1B | (SEQ ID NO: 5) | | | | |
| ROPN1 and | FQFLYTYIA | predicted | HLA-A2: 01 | 3.28E−06 | 1/4 co-cultures |
| ROPN1B | (SEQ ID NO: 6) | | | | |
| ROPN1 and | ALACSALGV | predicted | HLA-A2: 01 | 3.16E−06 | Not determined |
| ROPN1B | (SEQ ID NO: 7) | | | | |
| ROPN1 and | AQMWKVVNL | predicted | HLA-A2: 01 | 1.14E−05 | Not determined |
| ROPN1B | (SEQ ID NO: 8) | | | | |
| ROPN1 and | KMLKEFAKA | predicted | HLA-A2: 01 | 9.12E−05 | Not determined |
| ROPN1B | (SEQ ID NO: 9) | | | | |

### Example 2. Further Steps in Identifying and Validating Tumor-Restricted Antigen Targets for AT, their Epitopes, their Corresponding TCRs, and Engineered T Cells (Extension to Example 1)

Materials and Methods in Extension to Example 1

Generation and Culture of Cell Lines and T Cells

To make a ROPN1 or ROPN1B-overexpressing triple negative breast cancer (TNBC) cell, ROPN1+GFP or ROPN1B+GFP cDNA fragments (amino acid sequence accessible under UniProtKB Acc. No. Q9BZX4-1) (ROPN1-2A-GFP or ROPN1B-2A-GFP) were ordered via GeneArt (Regensburg, Germany), and amplified using PCR with gene-specific primers that included 15 bp extensions homologous to the PiggyBac PB510B-1 vector ends. The amplified fragments were cloned into PiggyBac vector (a kind gift from Dr. P. J. French, Erasmus M C, Rotterdam, the Netherlands) using the In Fusion cloning kit (Takara). Subsequently, the MDA-MB-231 cell line (ECACC catalogue no. 92020424, a cell line model for TNBC) was stably transfected with PiggyBac-ROPN1+GFP or ROPN1B+GFP DNA using Lipofectamine (Invitrogen) and Transposase Expression vector DNA (System Biosciences). The transfected MDA-MB-231 cell lines were FACSorted for GFP, after which expression of ROPN1 or ROPN1B was confirmed with PCR and immunohistochemical staining of cytospins (using gene-specific primers and an anti-ROPN1 antibody). Cells of the MDA-MB-231 wildtype and of its ROPN1 or ROPN1B-over-expressing variant were cultured in RPMI medium supplemented with 10% FBS, 200 mM L-glutamine and 1% antibiotics without and with 2 μg/mL puromycin, respectively. The packaging cell lines 293T and Phoenix-Ampho were cultured in DMEM supplemented with 10% FBS, 200 mM L-glutamine, nonessential amino acids, and 1% antibiotics (DMEM complete). T2 cells and BSM cells were cultured in RPMI medium supplemented with 10% FBS, 200 mM L-glutamine and 1% antibiotics.

Enrichment of T Cells

Enrichment of ROPN1 and 1B Epitope-Specific CD8 T Cells

Enrichment of epitope-specific T cells was performed by co-culturing naïve T cells with CD11c-positive cells that were loaded with peptide. PBMCs were passed through a cell strainer (70 μm) and used for the isolation of naïve T cells and CD11c cells. To isolate CD11c-positive cells (typically dendritic cells, DC), PBMCs were stained for 10 mM at 4° C. with Fc block (10 μL/10⁷ PBMCs, BD Pharmingen, Vianen, the Netherlands), after which cells were stained with CD11c-PE antibody (10 μL/10⁷ PBMCs, BD Pharmingen) for 30 mM at 4° C., washed, and incubated with PE beads (10 μL/10⁷ PBMCs, Miltenyi Biotech, Bergisch Gladbach, Germany) for another 15 mM at 4° C. After washing, cells were dissolved in magnetic-activated cell sorting (MACS) buffer and passed through a MACS LS column (Miltenyi Biotec) to positively select CD11c cells. After selection, CD11c cells were irradiated with 30 Gy, and cultured overnight in a 24-wells plate (1×10⁶/mL) in RPMI medium supplemented with 1% human serum (Sanquin), 1% antibiotics, 200 mM L-glutamine, a DC maturation cocktail (a mixture of GM-CSF (10 ng/mL, ImmunoTools, Germany), IL-4 (10 ng/mL, ImmunoTools), LPS (100 ng/mL, Invitrogen, Goteborg, Sweden) and IFN_{Y} (10 ng/mL, Preprotech, London, United Kingdom)), as well as peptide (10 pg/mL). Naïve T cells were isolated with use of the Naïve T Cell isolation kit (Miltenyi Biotec) according to the manufacturer's protocol, and suspended in RPMI medium supplemented with 5% human serum (Sanquin), 1% antibiotics, 200 mM L-glutamine and IL-7 (5 ng/mL, BD Pharmingen). The run-through of the naïve T cell selection was frozen down and used for re-stimulation of epitope-specific T cells. After maturation of CD11c cells, these cells were co-cultured with naïve T cells (1×10⁶/mL) in a 24-wells plate in the presence of low levels of IL-7 (5 ng/mL) for 72 h. At days 6 and 8, IL-7 and IL-15 (10 ng/mL) were added, and T cells were further cultured until day 12, after which these cells were rested for 24 h in the absence of stimulator cells. The next day, irradiated PBMCs loaded with peptide were added in a 1:1 ratio in the presence of IL-7 and IL-15 (re-stimulation 1). Enrichment cycles were performed 4 times (i.e., 3 re-stimulation cycles) using 2 to 7 donors per peptide.

Following enrichment, T cells were tested for ROPN1 and/or ROPN1B epitope-specific IFN_{Y} production. To this end, T2 cells (1×10⁶/mL) were loaded with peptide (20 ng/mL) for 30 min, after which T cells (1×10⁵) were cultured in a 1:1 ratio with T2 cells in a round bottom 96-wells plate for 18 h. Supernatant was collected and IFN-γ production was measured with an Enzyme-linked immunosorbent assay (ELISA, BioLegend) according to the manufacturer's protocol (FIG. 9A). T2 cells loaded with an irrelevant peptide were included as a negative control. T cell IFNg production was considered epitope-specific in case levels exceeded 200 pg/ml, and levels were minimally twice as high as for irrelevant peptide (see for full listing of criteria, FIG. 8). T cells that fulfilled these criteria were stained with peptide:MHC (pMHC) tetramers to determine the frequency of epitope-specific T cells. Empty Loadable HLA Tetramers (5 μL, Tetramer shop, Kongens Lyngby, Denmark) were incubated with 0.5 μL peptide (200 μM) for 30 min on ice. Complexes of pMHC were centrifuged (3300 g, 5 min) and incubated with 0.1×10⁶ T cells for 15 min at 37° C. in the dark. Next, an antibody cocktail containing CD3-FITC (1:30, BD) and CD8-APC (1:300, eBiosciences) was added and incubated for an additional 30 min at 4° C. in the dark. Finally, T cells were washed twice and fixed with 1% paraformaldehyde (PFA), after which events were acquired with FACSCelesta (BD) and analyzed using FlowJoX software. In case T cell binding of pMHC was observed in more than 0.5% of cells in the CD3-positive cell population (FIG. 8), then T cells were FACS-sorted with pMHC multimers (see FIG. 9B for examples).

TCR Cloning and Sequence Identification

In the next step, CD8 T cells were exposed to the SMARTer™ RACE cDNA Amplification Kit (Clontech) to identify ROPN1 and/or ROPN1B epitope-specific TCRα- and β-chains. In brief, RNA was isolated by spin column purification (NucleoSpin, Macherey-Nagel) after which 5′RACE-ready cDNA was made as in Kunert et al., J Immunol; 197(6):2541-2552 (2016), doi:10.4049/jimmunol.1502024, and PCR was performed to amplify TCR-V encoding regions. Initial products were re-amplified by nested PCRs, cloned into the TOPO 2.1 vector (Invitrogen), and subjected to DNA sequencing. TCRα and TCRβ sequences were verified in at least twelve colonies. Using the IMGT database and the HighV-QUEST tool (http://www.imgt.org), the TCR V, D, and J sequences were annotated according to the Lefranc nomenclature. In case for a given epitope, a TCRa or b sequence represented 30% or more of all functional sequences of the corresponding TCR chain (i.e., >30% clonal sequences, FIG. 8; examples in FIG. 9C), then those TCRs were matched with the other TCR chain(s) and used for gene transfer.

TCR Gene Identification and Transfer

TCRα and TCRβ genes were codon optimized (GeneArt, Regensburg, Germany) and cloned into the pMP71 vector (a kind gift of prof. Wolfgang Uckert, MDC, Berlin, Germany) using a TCR13-2A-TCRα cassette that was flanked by NotI and EcoRI restriction sites. Upon activation with anti-CD3 mAb OKT3, PBMCs from healthy donors were transduced with TCR-encoding retroviruses (pMP71) or empty vector that were produced by a co-culture of 293T and Phoenix-Ampho packaging cells, as described previously in Lamers et al., Cancer Gene Ther.;13(5):503-509 (2006), doi:10.1038/sj.cgt.7700916, and Straetemans G, Clin Dev Immunol, 2012.

Staining for surface-expressed TCR transgenes was performed as described above. In case TCR surface expression was observed in more than 5% of cells in the CD3-positive cell population (FIG. 8) in at least 2 donors, then TCRs were exposed to further testing (examples in FIG. 9D). Note that for a single epitope (EVI; SEQ ID NO:24) pMHC complexes were insensitive to detect TCR T cells and replaced by stainings with antibodies directed against TCR-Vb7.1 and CD137. The expression of CD137 was measured following 48 h stimulation with EVI epitope-loaded BSM cells, and threshold for inclusion again being expression in more than 5% of cells in the CD3-positive cell population in at least 2 donors. TCR T cells that fulfilled these criteria were MACS-sorted using pMHC complexes or according to up-regulated CD137 expression, and used for in vitro assays.

Testing TCRs for sensitivity and specificity

In a first series of in vitro tests, TCR-transduced T cells (6×10⁴/well in a 96-well plate) were co-cultured with ROPN1 or ROPN1B over-expressing MDA-MB231 tumor cells (2×10⁴/well) in a total volume of 200 μl of T cell medium for 24 h at 37° C. The ROPN1 or ROPN1B over-expressing tumor cells were generated as described under ‘generation and culture of cell lines and T cells’ (see above), and tumor cells were pre-treated 48 h with IFN-_{Y} before co-culture with T cells. T cell recognition of endogenously processed epitopes was demonstrated in case levels exceeded 200 pg/ml, and levels were minimally twice as high as for wt MDA-MB231 tumor cells (FIG. 8). T cells that fulfilled these criteria (examples in FIG. 10A) were assessed for their sensitivity towards their cognate epitope. To this end, TCR T cells were co-cultured with BSM cells loaded with peptide concentrations ranging from 1 pM to 30 μM to determine EC50 values (examples in FIG. 10B). EC50 values were calculated using GraphPad Prism 5 software.

Next, the recognition motifs of TCRs were determined using co-cultures between TCR T cells and BSM cells that were loaded with peptides (i.e., 10 μM) containing individual alanines as replacements at every single position in the cognate ROPN1 or ROPN1B epitope. Critical amino acid positions were defined as those that showed >50% decrease in IFN_{Y} production when alanine variant was compared to the cognate peptide. The resulting recognition motif was scanned for its occurrence in the humane proteome using the ScanProsite tool (https://prosite.expasy.org/scamprosite/) (examples in FIG. 11A). In addition, TCR T cells were screened for lack of reactivity towards 114 HLA-A2-eluted non-cognate peptides (examples in FIG. 11B). T cells transduced with the above-mentioned genes are also referred to as FLY-A, FLY-B and EVI epitope-specific TCRs.

Testing of TCRs in Advanced Models

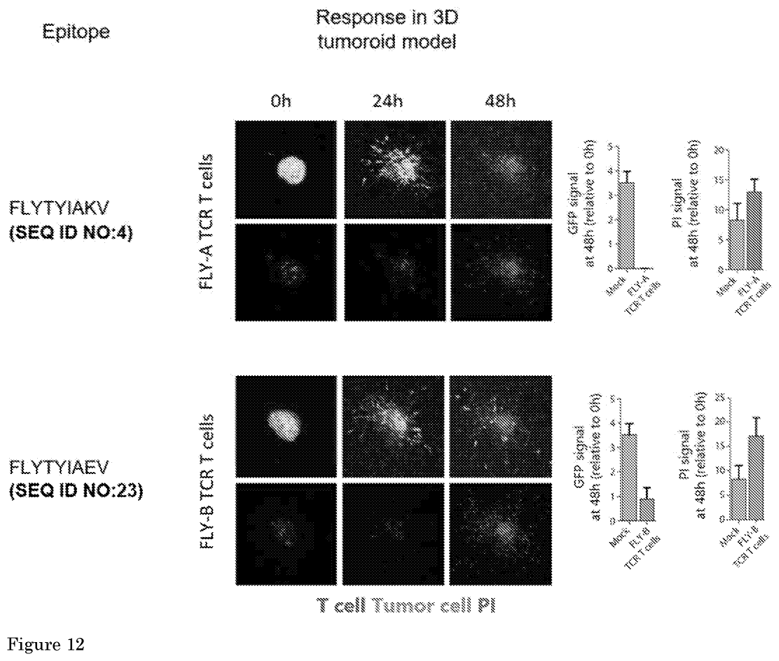

In a subsequent series of tests, TCR T cells were subjected to tracking and monitoring in a three-dimensional tumoroid model of breast cancer cells. Tumoroids were derived from ROPN1 or ROPN1B over-expressing MDA-MB231 tumor cells. A single tumor cell suspension was injected with a microinjector into a collagen-matrix to form a tumoroid overnight. TCR T cells were added directly on top of the tumoroid. Tumor cells expressed GFP (genetically coupled to ROPN1 or ROPN1B), TCR T cells were labeled with Hoechst prior to their addition to the tumoroid, and both tumor and T cells were PI-labeled to monitor cell death. At several time points after the addition of TCR T cells, images were recorded via fluorescent microscopy (examples in FIG. 12).

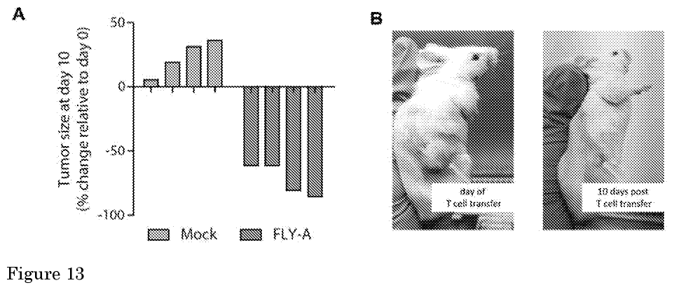

Finally, TCR T cells were tested for their anti-tumor efficacy in tumor-bearing immune-deficient mice. For this purpose, 2,5×10⁶ ROPN1 over-expressing MDA-MB231 tumor cells suspended in matrigel were sc transplanted in the right flank of NSG mice (NOD.Cg-Prkdc^{scid} Il2rg^{tm1Wjl}/SzJ, Charles River Laboratories, Paris, France). When tumors were palpable (˜200 mm³, 3-4 weeks following tumor transplant), mice were pretreated with busulfan (ip, 16.5 mg/kg, day −3) followed by cyclophosphamide (ip, 200 mg/kg, day −2). At day 0 and 3, mice received 2 iv transfers each of 15×10⁶ TCR or Mock (no TCR) human T cells. T cells were freshly transduced prior to day 0 transfer and maintained with 5 ng/ml IL-15 and IL-21 prior to day 3 transfer; and T cells were rested in the absence of cytokines for 24 h before T cell transfer. Mice received sc IL-2 injections (1×10⁵ IU) for 8 consecutive days following the second transfer of T cells. At 10 day 10, tumor regressions were measured relative to day 0, and compared for treatments with TCR versus Mock T cells (examples in FIG. 13).

Results in Extension to Example 1

Predicted and Eluted ROPN1 and ROPN1B Epitopes are Avidly Bound by HLA-A2

In extension to Example 1, experiments have been repeated with existing epitopes and experiments have started with new epitopes. FIG. 7 provides an overview of the current data from Example 1 and 2.

New ROPN1 or ROPN1B epitopes, when compared to Example 1, were either predicted using similar tools as described for Example 1, or searched in publicly available databases of eluted peptides, which yielded 10 additional epitopes. The total set of epitopes (n=28) were filtered according to unique occurrence in ROPN1 or ROPN1B antigen, and significant binding by HLA-A2 (overview presented in FIG. 7A). First, peptides were screened for non-homology to other peptide sequences present in the human proteome using the algorithm EXPITOPE, which provided a short list of 19 immunogenic, non-cross-reactive ROPN1 and/or ROPN1B peptides (i.e., peptides with >2 mismatches compared to any other peptide, Table 3). Second, these 19 peptides along with the reference and control peptides NY-ESO1 (SLLMWITQV) and gp100 (YLEPGPVTA) were tested for binding to HLA-A2 in vitro. Epitopes were considered bound by HLA-A2 in case: (1) binding stability was at least 1.1-fold higher when compared to no peptide (FIG. 7B); (2) EC50 was at least 5×10⁻⁵ M; and (3) binding amplitude was at least 50% of that of reference peptide YLE (FIGS. 7B and C). The remaining 11 epitopes were ranked according to amplitude values (FIG. 7D; SEQ ID NOs:1-9, 23, 24).

ROPN1 and ROPN1B Epitope-Specific CD8 T Cells are Enriched from Healthy Donor T Cells

The overall selection process, including the criteria epitopes and their corresponding TCRs need to adhere to, as well as the epitopes and/or their corresponding TCRs that pass each step, is schematically presented in FIG. 8, and described in Materials and Methods. We have used the 11 top-ranked epitopes to retrieve epitope-specific T cells. Following 4 enrichment cycles, T cells were tested for epitope-specific IFN_{Y} production and pMHC binding. Enrichment of epitope-specific T cells was demonstrated for 9 out of 11 ROPN1 or ROPN1B peptides (FIGS. 9A and B).

ROPN1 and ROPN1B Epitope-Specific TCRs are Functionally Expressed by T Cells

T cells specific for ROPN1 or ROPN1B epitopes were FACSorted using pMHC multimers, and used to identify TCR genes via 5′RACE PCR. For 6 out of 9 epitopes, the corresponding TCR genes demonstrated oligo or monoclonality (FIG. 9C), and matching TCR-ab combinations were codon-optimized, and cloned into pMP71. Surface expression in peripheral T cells was assessed according to binding of pMHC or, in case such pMHC complexes were not available, up-regulation of CD137 expression (for details see Materials and Methods). TCRab's specific for 5 out of 6 epitopes demonstrated functional expression (MLN, FLY-A, AQM, FLY-B, EVI; SEQ ID NOs: 1, 4, 8, 23, 24; FIG. 9D).

FLY-A, FLY-B and EVI Epitope-Specific TCRs Yield Sensitive and Specific T Cell Responses

The inventory of outcomes for all epitopes used for T cell enrichments, and/or their corresponding TCRs, is presented in Table 4. In a first and critical assay, TCR T cells have been tested for their reactivity against peptides that are processed and presented by tumor cells. TCRs that do not pass this step are most likely those TCRs that are specific for predicted non-natural peptides, and are excluded (quite early on in the selection process) from further testing. Our results showed that the FLY-A, FLY-B and EVI TCRs, but not the MLN and AQM TCRs, are able to mediate T cell IFNg upon stimulation with ROPN1 or ROPN1B expressing MDA-MB231 tumor cells (FIG. 10A). For the TCR T cells specific for FLY-A, FLY-B and EVI (SEQ ID NOs: 4, 23, 24), the sensitivity of the T cell response towards the cognate epitopes was determined according to dose titrations. The EC50 values were: 0.1, 1.2, and 18.1 mM for FLY-A, FLY-B and EVI TCR T cells, respectively, implying that in that order TCR T cells showed high to low avidity (FIG. 10B).

The encoded amino acid sequences of the transduced TCRα and TCRβ genes which passed the above assays are provided in SEQ ID NOs: 34 and 39 (binding FLY-A epitope/epitope 4), SEQ ID NOs: 47 and 52 (binding FLY-B epitope/epitope 10) and SEQ ID NOs:60 and 65 (binding EVI-epitope/epitope 11) and were used in subsequent assays.

In the next assays, the FLY-A and FLY-B TCR T cells were tested for their specificity towards the cognate epitopes; assays for EVI TCR T cells are ongoing. TCR T cells directed against either FLY epitope revealed that these epitopes harbor stringent recognition motifs (see for details Materials and Methods), namely: X-X-Y-T-Y-I-A-K-X (FLY-A) and X-L-Y-T-Y-I-A-E-X (FLY-B) (FIG. 11A). In fact, these motifs were not present in any other known sequence of the human proteome. In addition, both TCRs did not recognize HLA-A2 eluted non-cognate peptides (FIG. 11B).

FLY-A and FLY-B Epitope-Specific TCRs Yield Highly Effective T Cell Responses in Advanced Tumor Models

To further challenge the FLY-A and FLY-B TCR T cells, they were tested in 3D tumoroid models. Both TCRs mediated killing of ROPN1 or ROPN1B over-expressing MDA-MB231 breast cancer cells. This is exemplified by microscopic images showing loss of tumor cells (i.e., GFP signal) and increase in cell death (i.e., PI signal) after adding TCR T cells to the tumoroids, as well as by the quantifications of the GFP and PI signals in time (FIGS. 12A and B). Lastly, as an exemplary embodiment, the FLY-A TCR was tested in an immune-deficient mouse model. Mice that were bearing palpable subcutaneous tumors derived from ROPN1 over-expressing MDA-MB231 cells, and were treated with FLY-A TCR T cells, but not Mock T cells, showed reductions of tumor size at day 10 ranging from 60 to 90% (FIGS. 13A and B).

Discussion in Extension to Example 1

We present that selection of target antigen, epitopes as well as corresponding TCRs for adoptive T cell treatment requires a step-wise approach and stringent filtering according to criteria for therapeutic safety as well as efficacy. Along this line, ROPN1 and ROPN1B were selected as target antigens with an expression in TNBC that is selective (i.e., absent in normal tissues) and high and homogenous (i.e., clearly present in most if not all tumor cells) (see FIGS. 1 and 2). Besides solid tumors, such as TNBC and cutaneous melanoma, ROPN1/1B is also expressed in hematological malignancies, such as multiple myeloma. For example, we have found that the ROPN1(B) gene was expressed in up to 55% of bone-marrow samples of multiple myeloma patients in 5 different patient cohorts (data not shown). The epitopes from ROPN1 and ROPN1B were derived through predictions or immunopeptidomics, and filtered for uniqueness (i.e., not present in human proteome except for ROPN1 or ROPN1B) as well as binding properties towards HLA-A2 (see for overview of outcomes as well as criteria FIG. 7). This filtering shortlisted our original list of 28 epitopes down to 11 epitopes, which were used for enrichments of epitope-specific T cells as well as retrieval and testing of corresponding TCRs for sensitivity, specificity and anti-tumor efficacy in challenging in vitro and in vivo models (see for overview of outcomes as well as criteria FIG. 8). The filtering of TCRs led to the validation of 3 TCRab's out of more than 40 TCRab's with high therapeutic value; these TCRs are directed against the epitopes FLY-A, FLY-B or EVI (SEQ ID NOs: 4, 23, 24 (epitopes in aa); 33, 34, 38, 39, 46, 47, 51, 52, 59, 60, 64, 65 (TCRa and b in nt and aa sequences)).

Using a sensitive protocol enabling to retrieve epitope-specific T cells from low starting frequencies present in peripheral blood, we were able to detect enriched frequencies of T cells for 9 (out of 11) epitopes; of which we retrieved TCRab sequences for 6 epitopes; of which TCRab sequences for 5 epitopes were surface expressed when gene transferred into T cells (FIG. 9). These TCRab's were assessed for their reactivity against peptides that are processed and presented by tumor cells; in that way providing early exclusion of those TCRs that are specific for predicted non-natural peptides from further testing. Out of the 5 epitopes, the TCRs directed against the MLN and AQM epitopes did not, whereas the TCRs directed against the epitopes FLY-A, FLY-B or EVI did recognize endogenously processed epitopes (FIG. 10). It is noteworthy that Example 2 overrides earlier findings of Example 1 regarding MLN TCR T cells as multiple repetitions of this test demonstrated that this TCR did not mediate T cell IFNg production towards endogenously presented ROPN1B over-expressing tumor cells (9 out of 11 repetitions). The sensitivity of the FLY-A, FLY-B or EVI TCR T cells towards the cognate epitopes ranged from 0.1 (FLY-A) to 18 mM (EVI) (FIG. 10), with particularly the avidities of FLY-A or FLY-B TCR T cells being in the range of NY-ES01 TCR T cells (i.e., 0.7 mM) that has been used effectively clinically to treat melanoma and sarcoma patients (Robbins P, J Immunol, 2008; Robbins P, J Clin Oncol, 2011). Importantly, the specificities of FLY-A or FLY-B TCR T cells for their cognate epitopes according to their recognition motifs (i.e., 6 consecutive amino acids out of 9 are critical for recognition) as well as testing a library of non-cognate epitopes (FIG. 11). When challenging the remaining TCR T cells in more advanced tumor models, we demonstrated that the TCRs tested thus far (FLY-A TCR being the furthest) demonstrated significant ability to kill ROPN1-positive three-dimensional tumoroids in vitro as well as ROPN1-positive tumors when transplanted onto mice (FIGS. 12 and 13).

In short, we have identified and validated three TCRs directed against FLY-A, FLY-B and EVI epitopes of ROPN1 or ROPN1B that show high therapeutic value. See for TCR sequences SEQ ID Nos: 33, 34, 38, 39, 46, 47, 51, 52, 59, 60, 64, 65; and for annotated sequences FIG. 14. T cells gene-engineered with these TCRs are scheduled for their use in a trial to treat patients with TNBC or other ROPN1(B)-positive cancers.

Conclusion in Extension to Example 1

Established and utilized herein is an effective workflow to identify and validate tumor-restricted antigen targets for AT, their epitopes, their corresponding TCRs, and engineered T cells. Amongst other, we identified ROPN1 and ROPN1B as tumor targets for AT with absent expression in multiple healthy tissues, which implies minimal risk for on-target toxicity.

Also, we isolated 3 ROPN1 and/or ROPN1B-specific and HLA-A2-restricted TCRs that were expressed in peripheral T cells from healthy donors that mediate recognition of its endogenously presented epitopes epitope but not an irrelevant epitope. In addition, two of these TCRs (FLY-A and FLY-B TCR) showed a stringent recognition motif that is not present in any other human protein which imply minimal risk for off-target toxicities. Moreover, the FLY-A and FLY-B TCRs demonstrated clear anti-tumor efficacy in a 3-dimensional tumoroid model, and the FLY-A TCR demonstrated significant regression of tumors in a mouse model. With these results it is demonstrated that ROPN1 and ROPN1B represent an excellent target antigen and ROPN1 and/or ROPN1B-TCRs provide a novel treatment opportunity against cancers that display T cell epitopes of ROPN1 and/or ROPN1B, which is the case for >80% of TNBC patients.

TABLE 3
Overview of identified ROPN1 and ROPN1B epitopes,
and their non-cross reactivity (in bold)

| | | 0 | 1 | 2 |
|---|---|---|---|---|
| Soure | Peptides | mismatch | mismatch | mismatch |
| Predicted | ALACSALGV | No | No | No |
| | AQMWKVVNL | No | No | No |
| | DLFNSVMNV | No | No | 5 proteins |
| | ELTPELLKI | No | No | 3 proteins |
| | FLALACSAL | No | No | No |
| | FLYTYIAKV | No | No | No |
| | FLYTYIAEV | No | No | No |
| | FQFLYTYIA | No | No | No |
| | GLPRIPFST | No | No | No |
| | HVSRMLNYI | No | No | No |
| | KMLKEFAKA | No | No | No |
| | KTLKIVCEV | No | No | No |
| | LLKILHSQV | No | No | 1 protein |
| | LTPELLKIL | No | No | >5 proteins |
| | MLNYIEQEV | No | No | No |
| | RLIIRAEEL | No | No | No |
| | TITKTLKIV | No | No | 2 proteins |
| | YIAEVDGEI | No | No | No |
| Eluted | AELTPELLKI | No | No | No |
| | EISASHVSR | No | No | Yes |
| | EVIGPDGLITV | No | No | No |
| | FTQNPRVWL | No | No | 1 protein |
| | GVTITKTLK | No | No | No |
| | LIIRAEELAQM | No | No | No |
| | LPRIPFSTF | No | No | No |
| | LPTDLFNSV | No | No | 3 proteins |
| | SALGVTITK | No | No | No |
| | SPRIPFSTF | No | No | 2 proteins |

TABLE 4
Overview of identified ROPN1 and ROPNIB epitopes, their binding to HLA-A2 and their
immunogenicity

| | | | | | | | TCR | | | | T cell | | |
|---|---|---|---|---|---|---|---|---|---|---|---|---|---|
| | | | | | | | identification | | | | responses | | |
| | | | | | | pMHC | Monoclonality | | | | observed | | |
| | | | | Affinity | T cell responses | staining | (% of | | | Surface | (IFNg) against | | |
| | | | | for | observed (IFNg) | % pMHC+ | productive | | TCRs | expression | endogenously | | |
| | | | | HLA.A2.01 | against | CD8+ | sequences) | | # of | % pMHC+ in | presented | | motif |
| Protein | Epitopes | Source | HLA-subtype | (M) | epitope | in CD3+ | Alpha | Beta | TCRs | CD3+ | epitope | EC50 | Recognition |
| ROPN1B | MLNYIEQEV | predicted | HLA-A2: 01 | 4.82E−06 | 1/3 co-cultures | 13.3 | 50 | 100 | 2 | 49.7 | 0/2 responses | — | — |
| | (SEQ ID NO: 1) | | | | | | | | | | | | |
| ROPN1 and | FLALACSAL | predicted | HLA-A2: 01 | 4.50E−06 | 2/5 co-cultures | 0.077 | — | — | — | — | — | — | — |
| ROPN1B | (SEQ ID NO: 2) | | | | | | | | | | | | |
| ROPN1 and | KTLKIVCEV | predicted | HLA-A2: 01 | 4.40E−06 | 2/4 co-cultures | 0.28 | — | — | — | — | — | — | — |
| ROPN1B | (SEQ ID NO: 3) | | | | | | | | | | | | |
| ROPN1 | FLYTYIAKV | predicted/ | HLA-A2: 01 | 8.11E−06 | 1/4 co-cultures | 2.76 | 100 | 100 | 1 | 33.2 | 1/1 responses | 1.30E−007 | X-X-Y-T-Y- |
| | (SEQ ID NO: 4) | eluted | | | | | | | | | | | I-A-K-x |
| ROPN1 and | LIIRAEELAQM | eluted | HLA-A2: 01 | 6.99E−06 | 1/7 co-cultures | 0.60 | 31 | 15 | — | — | — | — | — |
| ROPN1B | (SEQ ID NO: 5) | | | | | | | | | | | | |
| ROPN1 and | FQFLYTYIA | predicted | HLA-A2: 01 | 3.43E−06 | 1/4 co-cultures | 0.43 | — | — | — | — | — | — | — |
| ROPN1B | (SEQ ID NO: 6) | | | | | | | | | | | | |
| ROPN1 and | ALACSALGV | predicted | HLA-A2: 01 | 2.84E−06 | 0/4 co-cultures | — | — | — | — | — | — | — | — |
| ROPN1B | (SEQ ID NO: 7) | | | | | | | | | | | | |
| ROPN1 and | AQMWKVVNL | predicted | HLA-A2: 01 | 1.05E−05 | 2/2 co-cultures | 2.58 | 100 | 100 | 1 | 49.7 | 0/1 responses | — | — |
| ROPN1B | (SEQ ID NO: 8) | | | | | | | | | | | | |
| ROPN1 and | KMLKEFAKA | predicted | HLA-A2: 01 | 3.82E−05 | 0/2 co-cultures | — | — | — | — | — | — | — | — |
| ROPN1B | (SEQ ID NO: 9) | | | | | | | | | | | | |
| ROPN1B | FLYTYIAEV | predicted | HLA-A2: 01 | 2.11E−05 | 2/2 co-cultures | 1.34 | 50 | 90 | 1 | 5.02 | 1/1 responses | 1.15E−006 | x-L-Y-T-Y- |
| | (SEQ ID NO: 23) | | | | | | | | | | | | I-A-E-x |
| ROPN1B | EVIGPDGLITV | eluted | HLA-A2: 01 | 6.55E−07 | 2/2 co-cultures | 8.25 | 50 | 40 | 7 | No pMHC | 1/7 responses | to be | to be |
| | (SEQ ID NO: 24) | | | | | | | | | | | determined | determined |

SEQUENCES

SEQ ID NO: 1: Epitope 1 (MLN)
MLNYIEQEV
SEQ ID NO: 2: Epitope 2
FLALACSAL
SEQ ID NO: 3: Epitope 3
KTLKIVCEV
SEQ ID NO: 4: Epitope 4 (FLY-A)
FLYTYIAKV
SEQ ID NO: 5: Epitope 5
LIIRAEELAQM
SEQ ID NO: 6: Epitope 6
FQFLYTYIA
SEQ ID NO: 7: Epitope 7
ALACSALGV
SEQ ID NO: 8: Epitope 8
AQMWKVVNL
SEQ ID NO: 9: Epitope 9
KMLKEFAKA
SEQ ID NO: 10: Epitope 1 (MLN)-specific TCR alpha chain non-
modified nucleotide sequence
ATGAAGACATTTGCTGGATTTTCGTTCCTGTTTTTGTGGCTGCAGCTGGACTGTATGAG
TAGAGGAGAGGATGTGGAGCAGAGTCTTTTCCTGAGTGTCCGAGAGGGAGACAGCTCCG
TTATAAACTGCACTTACACAGACAGCTCCTCCACCTACTTATACTGGTATAAGCAAGAA
CCTGGAGCAGGTCTCCAGTTGCTGACGTATATTTTTTCAAATATGGACATGAAACAAGA
CCAAAGACTCACTGTTCTATTGAATAAAAAGGATAAACATCTGTCTCTGCGCATTGCAG
ACACCCAGACTGGGGACTCAGCTATCTACTTCTGTGCAGAGGACGGAGGAGGAAGCTAC
ATACCTACATTTGGAAGAGGAACCAGCCTTATTGTTCATCCGTATATCCAGAACCCTGA
CCCTGCCGTGTACCAGCTGAGAGACTCTAAATCCAGTGACAAGTCTGTCTGCCTATTCA
CCGATTTTGATTCTCAAACAAATGTGTCACAAAGTAAGGATTCTGATGTGTATATCACA
GACAAAACTGTGCTAGACATGAGGTCTATGGACTTCAAGAGCAACAGTGCTGTGGCCTG
GAGCAACAAATCTGACTTTGCATGTGCAAACGCCTTCAACAACAGCATTATTCCAGAAG
ACACCTTCTTCCCCAGCCCAGAAAGTTCCTGTGATGTCAAGCTGGTCGAGAAAAGCTTT
GAAACAGATACGAACCTAAACTTTCAAAACCTGTCAGTGATTGGGTTCCGAATCCTCCT
CCTGAAAGTGGCCGGGTTTAATCTGCTCATGACGCTGCGGCTGTGGTCCAGCTGA
SEQ ID NO: 11: Epitope 1 (MLN)-specific TCR alpha chain amino
acid sequence
MKTFAGFSFLFLWLQLDCMSRGEDVEQSLFLSVREGDSSVINCTYTDSSSTYLYWYKQE
PGAGLQLLTYIFSNMDMKQDQRLTVLLNKKDKHLSLRIADTQTGDSAIYFCAEDGGGSY
IPTFGRGTSLIVHPYIQNPDPAVYQLRDSKSSDKSVCLFTDFDSQTNVSQSKDSDVYIT
DKTVLDMRSMDFKSNSAVAWSNKSDFACANAFNNSIIPEDTFFPSPESSCDVKLVEKSF
ETDTNLNFQNLSVIGFRILLLKVAGENLLMTLRLWSS
SEQ ID NO: 12: Epitope 1 (MLN)-specific TCR alpha chain CDR1
DSSSTY
SEQ ID NO: 13: Epitope 1 (MLN)-specific TCR alpha chain CDR2
IFSNMDM
SEQ ID NO: 14: Epitope 1 (MLN)-specific TCR alpha chain CDR3
AEDGGGSYIPT
SEQ ID NO: 15: Epitope 1 (MLN)-specific TCR beta chain non-
modified nucleotide sequence
ATGGTTTCCAGGCTTCTCAGTTTAGTGTCCCTTTGTCTCCTGGGAGCAAAGCACATAGA
AGCTGGAGTTACTCAGTTCCCCAGCCACAGCGTAATAGAGAAGGGCCAGACTGTGACTC
TGAGATGTGACCCAATTTCTGGACATGATAATCTTTATTGGTATCGACGTGTTATGGGA
AAAGAAATAAAATTTCTGTTACATTTTGTGAAAGAGTCTAAACAGGATGAATCCGGTAT
GCCCAACAATCGATTCTTAGCTGAAAGGACTGGAGGGACGTATTCTACTCTGAAGGTGC
AGCCTGCAGAACTGGAGGATTCTGGAGTTTATTTCTGTGCCAGCAGCCCCGGCCCTGGG
CAGAATTCACCCCTCCACTTTGGGAATGGGACCAGGCTCACTGTGACAGAGGACCTGAA
CAAGGTGTTCCCACCCGAGGTCGCTGTGTTTGAGCCATCAGAAGCAGAGATCTCCCACA
CCCAAAAGGCCACACTGGTGTGCCTGGCCACAGGCTTCTTCCCCGACCACGTGGAGCTG
AGCTGGTGGGTGAATGGGAAGGAGGTGCACAGTGGGGTCAGCACGGACCCGCAGCCCCT
CAAGGAGCAGCCCGCCCTCAATGACTCCAGATACTGCCTGAGCAGCCGCCTGAGGGTCT
CGGCCACCTTCTGGCAGAACCCCCGCAACCACTTCCGCTGTCAAGTCCAGTTCTACGGG
CTCTCGGAGAATGACGAGTGGACCCAGGATAGGGCCAAACCCGTCACCCAGATCGTCAG
CGCCGAGGCCTGGGGTAGAGCAGACTGTGGCTTTACCTCGGTGTCCTACCAGCAAGGGG
TCCTGTCTGCCACCATCCTCTATGAGATCCTGCTAGGGAAGGCCACCCTGTATGCTGTG
CTGGTCAGCGCCCTTGTGTTGATGGCCATGGTCAAGAGAAAGGATTTCTGA.
SEQ ID NO: 16: Epitope 1 (MLN)-specific TCR beta chain amino acid
sequence
MVSRLLSLVSLCLLGAKHIEAGVTQFPSHSVIEKGQTVTLRCDPISGHDNLYWYRRVMG
KEIKFLLHFVKESKQDESGMPNNRFLAERTGGTYSTLKVQPAELEDSGVYFCASSPGPG
QNSPLHFGNGTRLTVTEDLNKVFPPEVAVFEPSEAEISHTQKATLVCLATGFFPDHVEL
SWWVNGKEVHSGVSTDPQPLKEQPALNDSRYCLSSRLRVSATFWQNPRNHERCQVQFYG
LSENDEWTQDRAKPVTQIVSAEAWGRADCGFTSVSYQQGVLSATILYEILLGKATLYAV
LVSALVLMAMVKRKDF
SEQ ID NO: 17: Epitope 1 (MLN)-specific TCR beta chain CDR1
SGHDN
SEQ ID NO: 18: Epitope 1 (MLN)-specific TCR beta chain CDR2
FVKESK
SEQ ID NO: 19: Epitope 1 (MLN)-specific TCR beta chain CDR3
ASSPGPGQNSPLH
SEQ ID NO: 20: Motif epitope 1 (MLN)
MLNYIXQXX
SEQ ID NO: 21: TRAV and TRAJ domains of SEQ ID NO: 11
GEDVEQSLFLSVREGDSSVINCTYTDSSSTYLYWYKQEPGAGLQLLTYIFSNMDMKQDQ
RLTVLLNKKDKHLSLRIADTQTGDSAIYFCAEDGGGSYIPTFGRGTSLIVHP
SEQ ID NO: 22: TRBV, TRBD and TRBJ domains of SEQ ID NO16
EAGVTQFPSHSVIEKGQTVTLRCDPISGHDNLYWYRRVMGKEIKFLLHFVKESKQDESG
MPNNRFLAERTGGTYSTLKVQPAELEDSGVYFCASSPGPGQNSPLHFGNGTRLTVT
SEQ ID NO: 23: Epitope 10 (FLY-B)
FLYTYIAEV
SEQ ID NO: 24: Epitope 11 (EVI)
EVIGPDGLITV
SEQ ID NO: 25: Epitope 12
GLPRIPEST
SEQ ID NO: 26: Epitope 13
HVSRMLNYI
SEQ ID NO: 27: Epitope 14
RLIIRAEEL
SEQ ID NO: 28: Epitope 15
YIEVDGEI
SEQ ID NO: 29: Epitope 16
AELTPELLKI
SEQ ID NO: 30: Epitope 17
GVTITKTLK
SEQ ID NO: 31: Epitope 18
LPRIPESTE
SEQ ID NO: 32: Epitope 19
SALGVTITK
SEQ ID NO: 33: Epitope 4 (FLY-A)-specific TCR alpha chain non-
modified nucleotide sequence
atgatgaaatccttgagagttttactagtgatcctgtggcttcagttgagctgggtttg
gagccaacagaaggaggtggagcagaattctggacccctcagtgttccagagggagcca
ttgcctctctcaactgcacttacagtgaccgaggttcccagtccttcttctggtacaga
caatattctgggaaaagccctgagttgataatgtccatatactccaatggtgacaaaga
agatggaaggtttacagcacagctcaataaagccagccagtatgtttctctgctcatca
gagactcccagcccagtgattcagccacctacctctgtgccgtgaacggggatagcagc
tataaattgatcttcgggagtgggaccagactgctggtcaggcctgATATCCAGAACCC
TGACCCTGCCGTGTACCAGCTGAGAGACTCTAAATCCAGTGACAAGTCTGTCTGCCTAT
TCACCGATTTTGATTCTCAAACAAATGTGTCACAAAGTAAGGATTCTGATGTGTATATC
ACAGACAAAACTGTGCTAGACATGAGGTCTATGGACTTCAAGAGCAACAGTGCTGTGGC
CTGGAGCAACAAATCTGACTTTGCATGTGCAAACGCCTTCAACAACAGCATTATTCCAG
AAGACACCTTCTTCCCCAGCCCAGAAAGTTCCTGTGATGTCAAGCTGGTCGAGAAAAGC
TTTGAAACAGATACGAACCTAAACTTTCAAAACCTGTCAGTGATTGGGTTCCGAATCCT
CCTCCTGAAAGTGGCCGGGTTTAATCTGCTCATGACGCTGCGGCTGTGGTCCAGCTGA
SEQ ID NO: 34: Epitope 4 (FLY-A)-specific TCR alpha chain amino
acid sequence
MMKSLRVLLVILWLQLSWVWSQQKEVEQNSGPLSVPEGAIASLNCTYSDRGSQSFFWYR
QYSGKSPELIMSIYSNGDKEDGRFTAQLNKASQYVSLLIRDSQPSDSATYLCAVNGDSS
YKLIFGSGTRLLVRPDIQNPDPAVYQLRDSKSSDKSVCLFTDFDSQTNVSQSKDSDVYI
TDKTVLDMRSMDFKSNSAVAWSNKSDFACANAFNNSIIPEDTFFPSPESSCDVKLVEKS
FETDTNLNFQNLSVIGFRILLLKVAGENLLMTLRLWSS
SEQ ID NO: 35: Epitope 4 (FLY-A)-specific TCR alpha chain CDR1
DRGSQS
SEQ ID NO: 36: Epitope 4 (FLY-A)-specific TCR alpha chain CDR2
IYSNGD
SEQ ID NO: 37: Epitope 4 (FLY-A)-specific TCR alpha chain CDR3
AVNGDSSYKLI
SEQ ID NO: 38: Epitope 4 (FLY-A)-specific TCR beta chain non-
modified nucleotide sequence
atgagcatcggcctcctgtgctgtgcagccttgtctctcctgtgggcaggtccagtgaa
tgctggtgtcactcagaccccaaaattccaggtcctgaagacaggacagagcatgacac
tgcagtgtgcccaggatatgaaccatgaatacatgtcctggtatcgacaagacccaggc
atggggctgaggctgattcattactcagttggtgctggtatcactgaccaaggagaagt
ccccaatggctacaatgtctccagatcaaccacagaggatttcccgctcaggctgctgt
cggctgctccctcccagacatctgtgtacttctgtgccagcagttactccctaggggat
ggctacaccttcggttcggggaccaggttaaccgttgtagAGGACCTGAACAAGGTGTT
CCCACCCGAGGTCGCTGTGTTTGAGCCATCAGAAGCAGAGATCTCCCACACCCAAAAGG
CCACACTGGTGTGCCTGGCCACAGGCTTCTTCCCCGACCACGTGGAGCTGAGCTGGTGG
GTGAATGGGAAGGAGGTGCACAGTGGGGTCAGCACGGACCCGCAGCCCCTCAAGGAGCA
GCCCGCCCTCAATGACTCCAGATACTGCCTGAGCAGCCGCCTGAGGGTCTCGGCCACCT
TCTGGCAGAACCCCCGCAACCACTTCCGCTGTCAAGTCCAGTTCTACGGGCTCTCGGAG
AATGACGAGTGGACCCAGGATAGGGCCAAACCCGTCACCCAGATCGTCAGCGCCGAGGC
CTGGGGTAGAGCAGACTGTGGCTTTACCTCGGTGTCCTACCAGCAAGGGGTCCTGTCTG
CCACCATCCTCTATGAGATCCTGCTAGGGAAGGCCACCCTGTATGCTGTGCTGGTCAGC
GCCCTTGTGTTGATGGCCATGGTCAAGAGAAAGGATTTCTGA
SEQ ID NO: 39: Epitope 4 (FLY-A)-specific TCR beta chain amino
acid sequence
MSIGLLCCAALSLLWAGPVNAGVTQTPKFQVLKTGQSMTLQCAQDMNHEYMSWYRQDPG
MGLRLIHYSVGAGITDQGEVPNGYNVSRSTTEDFPLRLLSAAPSQTSVYFCASSYSLGD
GYTFGSGTRLTVVEDLNKVFPPEVAVFEPSEAEISHTQKATLVCLATGFFPDHVELSWW
VNGKEVHSGVSTDPQPLKEQPALNDSRYCLSSRLRVSATFWQNPRNHFRCQVQFYGLSE
NDEWTQDRAKPVTQIVSAEAWGRADCGFTSVSYQQGVLSATILYEILLGKATLYAVLVS
ALVLMAMVKRKDF
SEQ ID NO: 40: Epitope 4 (FLY-A)-specific TCR beta chain CDR1
MNHEY
SEQ ID NO: 41: Epitope 4 (FLY-A)-specific TCR beta chain CDR2
SVGAGI
SEQ ID NO: 42: Epitope 4 (FLY-A)-specific TCR beta chain CDR3
ASSYSLGDGYT
SEQ ID NO: 43: Motif epitope 4 (FLY-A)
XXYTYIAKX
SEQ ID NO: 44: TRAV and TRAJ domains of SEQ ID NO: 34
QKEVEQNSGPLSVPEGAIASLNCTYSDRGSQSFFWYRQYSGKSPELIMSIYSNGDKEDG
RFTAQLNKASQYVSLLIRDSQPSDSATYLCAVNGDSSYKLIFGSGTRLLVRP
SEQ ID NO: 45: TRBV, TRBD and TRBJ domains of SEQ ID NO: 39
NAGVTQTPKFQVLKTGQSMTLQCAQDMNHEYMSWYRQDPGMGLRLIHYSVGAGITDQGE
VPNGYNVSRSTTEDFPLRLLSAAPSQTSVYFCASSYSLGDGYTFGSGTRLTVV
SEQ ID NO: 46: Epitope 10 (FLY-B)-specific TCR alpha chain non-
modified nucleotide sequence
atggaaactctcctgggagtgtctttggtgattctatggcttcaactggctagggtgaa
cagtcaacagggagaagaggatcctcaggccttgagcatccaggagggtgaaaatgcca
ccatgaactgcagttacaaaactagtataaacaatttacagtggtatagacaaaattca
ggtagaggccttgtccacctaattttaatacgttcaaatgaaagagagaaacacagtgg
aagattaagagtcacgcttgacacttccaagaaaagcagttccttgttgatcacggctt
cccgggcagcagacactgcttcttacttctgtgctacggacgctagggccagactcatg
tttggagatggaactcagctggtggtgaagcccaATATCCAGAACCCTGACCCTGCCGT
GTACCAGCTGAGAGACTCTAAATCCAGTGACAAGTCTGTCTGCCTATTCACCGATTTTG
ATTCTCAAACAAATGTGTCACAAAGTAAGGATTCTGATGTGTATATCACAGACAAAACT
GTGCTAGACATGAGGTCTATGGACTTCAAGAGCAACAGTGCTGTGGCCTGGAGCAACAA
ATCTGACTTTGCATGTGCAAACGCCTTCAACAACAGCATTATTCCAGAAGACACCTTCT
TCCCCAGCCCAGAAAGTTCCTGTGATGTCAAGCTGGTCGAGAAAAGCTTTGAAACAGAT
ACGAACCTAAACTTTCAAAACCTGTCAGTGATTGGGTTCCGAATCCTCCTCCTGAAAGT
GGCCGGGTTTAATCTGCTCATGACGCTGCGGCTGTGGTCCAGCTGa
SEQ ID NO: 47: Epitope 10 (FLY-B)-specific TCR alpha chain amino
acid sequence
METLLGVSLVILWLQLARVNSQQGEEDPQALSIQEGENATMNCSYKTSINNLQWYRQNS
GRGLVHLILIRSNEREKHSGRLRVTLDTSKKSSSLLITASRAADTASYFCATDARARLM
FGDGTQLVVKPNIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVKLVEKSFETD
TNLNFQNLSVIGFRILLLKVAGENLLMTLRLWSS
SEQ ID NO: 48: Epitope 10 (FLY-B)-specific TCR alpha chain CDR1
TSINN
SEQ ID NO: 49: Epitope 10 (FLY-B)-specific TCR alpha chain CDR2
IRSNERE
SEQ ID NO: 50: Epitope 10 (FLY-B)-specific TCR alpha chain CDR3
ATDARARLM
SEQ ID NO: 51: Epitope 10( FLY-B)-specific TCR beta chain non-
modified nucleotide sequence
atggactcctggaccctctgctgtgtgtccctttgcatcctggtagcaaagcacacaga
tgctggagttatccagtcaccccggcacgaggtgacagagatgggacaagaagtgactc
tgagatgtaaaccaatttcaggacacgactaccttttctggtacagacagaccatgatg
cggggactggagttgctcatttactttaacaacaacgttccgatagatgattcagggat
gcccgaggatcgattctcagctaagatgcctaatgcatcattctccactctgaagatcc
agccctcagaacccagggactcagctgtgtacttctgtgccagcagtttgggggggggg
acgaggcccctacctaattcacccctccactttgggaacgggaccaggctcactgtgac
agAGGACCTGAACAAGGTGTTCCCACCCGAGGTCGCTGTGTTTGAGCCATCAGAAGCAG
AGATCTCCCACACCCAAAAGGCCACACTGGTGTGCCTGGCCACAGGCTTCTTCCCCGAC
CACGTGGAGCTGAGCTGGTGGGTGAATGGGAAGGAGGTGCACAGTGGGGTCAGCACGGA
CCCGCAGCCCCTCAAGGAGCAGCCCGCCCTCAATGACTCCAGATACTGCCTGAGCAGCC
GCCTGAGGGTCTCGGCCACCTTCTGGCAGAACCCCCGCAACCACTTCCGCTGTCAAGTC
CAGTTCTACGGGCTCTCGGAGAATGACGAGTGGACCCAGGATAGGGCCAAACCCGTCAC
CCAGATCGTCAGCGCCGAGGCCTGGGGTAGAGCAGACTGTGGCTTTACCTCGGTGTCCT
ACCAGCAAGGGGTCCTGTCTGCCACCATCCTCTATGAGATCCTGCTAGGGAAGGCCACC
CTGTATGCTGTGCTGGTCAGCGCCCTTGTGTTGATGGCCATGGTCAAGAGAAAGGATTT
Ctga
SEQ ID NO: 52: Epitope 10 (FLY-B)-specific TCR beta chain amino
acid sequence
MDSWTLCCVSLCILVAKHTDAGVIQSPRHEVTEMGQEVTLRCKPISGHDYLFWYRQTMM
RGLELLIYFNNNVPIDDSGMPEDRFSAKMPNASESTLKIQPSEPRDSAVYFCASSLGGG
TRPLPNSPLHFGNGTRLTVTEDLNKVFPPEVAVFEPSEAEISHTQKATLVCLATGFFPD
HVELSWWVNGKEVHSGVSTDPQPLKEQPALNDSRYCLSSRLRVSATFWQNPRNHERCQV
QFYGLSENDEWTQDRAKPVTQIVSAEAWGRADCGFTSVSYQQGVLSATILYEILLGKAT
LYAVLVSALVLMAMVKRKDF
SEQ ID NO: 53: Epitope 10 (FLY-B)-specific TCR beta chain CDR1
SGHDY
SEQ ID NO: 54: Epitope 10 (FLY-B)-specific TCR beta chain CDR2
FNNNVP
SEQ ID NO: 55: Epitope 10 (FLY-B)-specific TCR beta chain CDR3
ASSLGGGTRPLPNSPLH
SEQ ID NO: 56: Motif Epitope 10 (FLY-B)
XLYTYIAEX
SEQ ID NO: 57: TRAV and TRAJ domains of SEQ ID NO: 47
SQQGEEDPQALSIQEGENATMNCSYKTSINNLQWYRQNSGRGLVHLILIRSNEREKHSG
RLRVTLDTSKKSSSLLITASRAADTASYFCATDARARLMFGDGTQLVVKP
SEQ ID NO: 58: TRBV, TRBD and TRBJ domains of SEQ ID NO: 52
DAGVIQSPRHEVTEMGQEVTLRCKPI SGHDYLFWYRQTMMRGLELLIYENNNVPIDDSG
MPEDRFSAKMPNASESTLKIQPSEPRDSAVYFCASSLGGGTRPLPNSPLHFGNGTRLTV
T
SEQ ID NO: 59: Epitope 11 (EVI)-specific TCR alpha chain non-
modified nucleotide sequence
atgctcctgctgctcgtcccagcgttccaggtgatttttaccctgggaggaaccagagc
ccagtctgtgacccagcttgacagccaagtccctgtctttgaagaagcccctgtggagc
tgaggtgcaactactcatcgtctgtttcagtgtatctcttctggtatgtgcaatacccc
aaccaaggactccagcttctcctgaagtatttatcaggatccaccctggttaaaggcat
caacggttttgaggctgaatttaacaagagtcaaacttccttccacttgaggaaaccct
cagtccatataagcgacacggctgagtacttctgtgctgctcggacgggaggaggaaac
aaactcacctttgggacaggcactcagctaaaagtggaactcaATATCCAGAACCCTGA
CCCTGCCGTGTACCAGCTGAGAGACTCTAAATCCAGTGACAAGTCTGTCTGCCTATTCA
CCGATTTTGATTCTCAAACAAATGTGTCACAAAGTAAGGATTCTGATGTGTATATCACA
GACAAAACTGTGCTAGACATGAGGTCTATGGACTTCAAGAGCAACAGTGCTGTGGCCTG
GAGCAACAAATCTGACTTTGCATGTGCAAACGCCTTCAACAACAGCATTATTCCAGAAG
ACACCTTCTTCCCCAGCCCAGAAAGTTCCTGTGATGTCAAGCTGGTCGAGAAAAGCTTT
GAAACAGATACGAACCTAAACTTTCAAAACCTGTCAGTGATTGGGTTCCGAATCCTCCT
CCTGAAAGTGGCCGGGTTTAATCTGCTCATGACGCTGCGGCTGTGGTCCAGCTGA
SEQ ID NO: 60: Epitope 11 (EVI)-specific TCR alpha chain amino
acid sequence
MLLLLVPAFQVIFTLGGTRAQSVTQLDSQVPVFEEAPVELRCNYSSSVSVYLFWYVQYP
NQGLQLLLKYLSGSTLVKGINGFEAEFNKSQTSFHLRKPSVHISDTAEYFCAARTGGGN
KLTFGTGTQLKVELNIQNPDPAVYQLRDSKSSDKSVCLFTDFDSQTNVSQSKDSDVYIT
DKTVLDMRSMDFKSNSAVAWSNKSDFACANAFNNSIIPEDTFFPSPESSCDVKLVEKSF
ETDTNLNFQNLSVIGFRILLLKVAGENLLMTLRLWSS
SEQ ID NO: 61: Epitope 11 (EVI)-specific TCR alpha chain CDR1
SSVSVY
SEQ ID NO: 62: Epitope 11 (EVI)-specific TCR alpha chain CDR2
YLSGSTLV
SEQ ID NO: 63: Epitope 11 (EVI)-specific TCR alpha chain CDR3
AARTGGGNKLT
SEQ ID NO: 64: Epitope 11 (EVI)-specific TCR beta chain non-
modified nucleotide sequence
atgggctgcaggctgctctgctgtgcggttctctgtctcctgggagcagttcccataga
cactgaagttacccagacaccaaaacacctggtcatgggaatgacaaataagaagtctt
tgaaatgtgaacaacatatggggcacagggctatgtattggtacaagcagaaagctaag
aagccaccggagctcatgtttgtctacagctatgagaaactctctataaatgaaagtgt
gccaagtcgcttctcacctgaatgccccaacagctctctcttaaaccttcacctacacg
ccctgcagccagaagactcagccctgtatctctgcgccagcagccaagaagggctagcg
ggagtaccccagtacttcgggccaggcacgcggctcctggtgctcgAGGACCTGAAAAA
CGTGTTCCCACCCGAGGTCGCTGTGTTTGAGCCATCAGAAGCAGAGATCTCCCACACCC
AAAAGGCCACACTGGTGTGCCTGGCCACAGGCTTCTACCCCGACCACGTGGAGCTGAGC
TGGTGGGTGAATGGGAAGGAGGTGCACAGTGGGGTCAGCACAGACCCGCAGCCCCTCAA
GGAGCAGCCCGCCCTCAATGACTCCAGATACTGCCTGAGCAGCCGCCTGAGGGTCTCGG
CCACCTTCTGGCAGAACCCCCGCAACCACTTCCGCTGTCAAGTCCAGTTCTACGGGCTC
TCGGAGAATGACGAGTGGACCCAGGATAGGGCCAAACCTGTCACCCAGATCGTCAGCGC
CGAGGCCTGGGGTAGAGCAGACTGTGGCTTCACCTCCGAGTCTTACCAGCAAGGGGTCC
TGTCTGCCACCATCCTCTATGAGATCTTGCTAGGGAAGGCCACCTTGTATGCCGTGCTG
GTCAGTGCCCTCGTGCTGATGGCCATGGTCAAGAGAAAGGATTCCAGAGGCtga
SEQ ID NO: 65: Epitope 11 (EVI)-specific TCR beta chain amino acid
sequence
MGCRLLCCAVLCLLGAVPIDTEVTQTPKHLVMGMTNKKSLKCEQHMGHRAMYWYKQKAK
KPPELMFVYSYEKLSINESVPSRESPECPNSSLLNLHLHALQPEDSALYLCASSQEGLA
GVPQYFGPGTRLLVLEDLKNVFPPEVAVFEPSEAEISHTQKATLVCLATGFYPDHVELS
WWVNGKEVHSGVSTDPQPLKEQPALNDSRYCLSSRLRVSATFWQNPRNHERCQVQFYGL
SENDEWTQDRAKPVTQIVSAEAWGRADCGFTSESYQQGVLSATILYEILLGKATLYAVL
VSALVLMAMVKRKDSRG
SEQ ID NO: 66: Epitope 11 (EVI)-specific TCR beta chain CDR1
MGHRA
SEQ ID NO: 67: Epitope 11 (EVI)-specific TCR beta chain CDR2
YSYEKL
SEQ ID NO: 68: Epitope 11 (EVI)-specific TCR beta chain CDR3
ASSQEGLAGVPQY
SEQ ID NO: 69: TRAV and TRAJ domains of SEQ ID NO: 60
AQSVTQLDSQVPVFEEAPVELRCNYSSSVSVYLFWYVQYPNQGLQLLLKYLSGSTLVKG
INGFEAEFNKSQTS FHLRKPSVHISDTAEYFCAARTGGGNKLTFGTGTQLKVEL
SEQ ID NO: 70: TRBV, TRBD and TRBJ domains of SEQ ID NO: 65
DTEVTQTPKHLVMGMTNKKSLKCEQHMGHRAMYWYKQKAKKPPELMFVYSYEKLSINES
VPSRFSPECPNSSLLNLHLHALQPEDSALYLCASSQEGLAGVPQYFGPGTRLLVL

1. An engineered T cell, wherein said T cell is engineered to express a T cell receptor (TCR) or an antibody-based receptor that binds to a T cell epitope of human ropporin-1A (ROPN1) and/or human ropporin-1B (ROPN1B); wherein said T cell epitope consists of the amino acid sequence selected from one of SEQ ID NO:4, SEQ ID NO:43, SEQ ID NO:23, SEQ ID NO:56 and SEQ ID NO:24.

2. The engineered T cell according to claim 1, wherein said T cell is engineered to express a TCR that binds to a T cell epitope consisting of SEQ ID NO:4 and/or SEQ ID NO:43; and wherein said TCR comprises:
(i) a T cell receptor alpha chain comprising a hypervariable region that comprises a CDR3 of SEQ ID NO:37, and
(ii) a T cell receptor beta chain comprising a hypervariable region that comprises a CDR3 of SEQ ID NO:42;
and wherein said hypervariable regions of said T cell receptor alpha and beta chain further comprise a CDR1 and a CDR2.

3. The engineered T cell according to claim 2, wherein said hypervariable region of said T cell receptor alpha chain comprises:
a CDR1 of SEQ ID NO:35;
a CDR2 of SEQ ID NO:36;
a CDR3 of SEQ ID NO:37; and
wherein said hypervariable region of said T cell receptor beta chain comprises:
a CDR1 of SEQ ID NO:40;
a CDR2 of SEQ ID NO:41;
a CDR3 of SEQ ID NO:42.

4. The engineered T cell according to claim 2, wherein said T cell receptor alpha chain comprises an amino acid sequence of SEQ ID NO:44 and wherein said T cell receptor beta chain comprises an amino acid sequence of SEQ ID NO:45; preferably wherein said T cell receptor alpha chain comprises an amino acid sequence of SEQ ID NO:34 and wherein said T cell receptor beta chain comprises an amino acid sequence of SEQ ID NO:39.

5. The engineered T cell according to claim 1, wherein said T cell is engineered to express a TCR that binds to a T cell epitope consisting of SEQ ID NO:23 and/or SEQ ID NO:56; and wherein said TCR comprises:
(i) a T cell receptor alpha chain comprising a hypervariable region that comprises a CDR3 of SEQ ID NO:50, and
(ii) a T cell receptor beta chain comprising a hypervariable region that comprises a CDR3 of SEQ ID NO:55;
and wherein said hypervariable regions of said T cell receptor alpha and beta chain further comprise a CDR1 and a CDR2.

6. The engineered T cell according to claim 5, wherein said hypervariable region of said T cell receptor alpha chain comprises:
a CDR1 of SEQ ID NO:48;
a CDR2 of SEQ ID NO:49;
a CDR3 of SEQ ID NO:50; and
wherein said hypervariable region of said T cell receptor beta chain comprises:
a CDR1 of SEQ ID NO:53;
a CDR2 of SEQ ID NO:54;
a CDR3 of SEQ ID NO:55.

7. The engineered T cell according to claim 5, wherein said T cell receptor alpha chain comprises an amino acid sequence of SEQ ID NO:57 and wherein said T cell receptor beta chain comprises an amino acid sequence of SEQ ID NO:58; preferably wherein said T cell receptor alpha chain comprises an amino acid sequence of SEQ ID NO:47 and wherein said T cell receptor beta chain comprises an amino acid sequence of SEQ ID NO:52.

8. The engineered T cell according to claim 1, wherein said T cell is engineered to express a TCR that binds to a T cell epitope of SEQ ID NO:24; and wherein said TCR comprises:
(i) a T cell receptor alpha chain comprising a hypervariable region that comprises a CDR3 of SEQ ID NO:63, and
(ii) a T cell receptor beta chain comprising a hypervariable region that comprises a CDR3 of SEQ ID NO:68;
and wherein said hypervariable regions of said T cell receptor alpha and beta chain further comprise a CDR1 and a CDR2.

9. The engineered T cell according to claim 8, wherein said hypervariable region of said T cell receptor alpha chain comprises:
a CDR1 of SEQ ID NO:61;
a CDR2 of SEQ ID NO:62;
a CDR3 of SEQ ID NO:63; and
wherein said hypervariable region of said T cell receptor beta chain comprises:
a CDR1 of SEQ ID NO:66;
a CDR2 of SEQ ID NO:67;
a CDR3 of SEQ ID NO:68.

10. The engineered T cell according to claim 8, wherein said T cell receptor alpha chain comprises an amino acid sequence of SEQ ID NO:69 and wherein said T cell receptor beta chain comprises an amino acid sequence of SEQ ID NO:70; preferably wherein said T cell receptor alpha chain comprises an amino acid sequence of SEQ ID NO:60 and wherein said T cell receptor beta chain comprises an amino acid sequence of SEQ ID NO:65.

11. The engineered T cell according to claim 1, wherein said T cell epitope forms a complex with a human Major Histocompatibility Complex (MHC) molecule, preferably an HLA-A*02 molecule.

12. A pharmaceutical composition comprising an engineered T cell according to claim 1, and a pharmaceutically acceptable excipient.

13. A TCR protein or antibody-based receptor protein, wherein said TCR protein or antibody-based receptor protein comprises a TCR or antibody-based receptor as defined in claim 1; preferably wherein said TCR has a T cell receptor alpha chain and a T cell receptor beta chain as defined in claim 2; preferably wheren said TCR protein or antibody-based receptor protein is part of an antibody drug conjugate (ADC) or is (part of) a soluble TCR.

14. A nucleic acid molecule comprising a nucleic acid sequence that encodes a T cell receptor alpha chain as defined in claim 2 and/or a T cell receptor beta chain as defined in claim 2.

15. An engineered T cell according to claim 1, a composition according to claim 12, a TCR protein or antibody-based receptor protein according to claim 13 or a nucleic acid molecule according to claim 14, for use in therapy.

16. The engineered T cell, composition, TCR protein, antibody-based receptor protein or nucleic acid molecule for use according to claim 15, wherein said engineered T cell, composition, TCR protein, antibody-based receptor protein or nucleic acid molecule are for use in the treatment of a tumor, preferably a solid tumor or a liquid tumor.

17. The engineered T cell, composition, TCR protein, antibody-based receptor protein or nucleic acid molecule for use according to claim 16, wherein said tumor comprises tumor cells expressing human ROPN1 and/or human ROPN1B, preferably wherein said tumor comprises tumor cells that comprise an MHC molecule that is in complex with, or bound to, a T cell epitope as defined in claim 1.

18. The engineered T cell, composition, TCR protein, antibody-based receptor protein or nucleic acid molecule for use according to claim 16 or 17, wherein said solid tumor is a breast cancer, preferably a triple negative breast cancer, or a skin cancer, preferably a melanoma.

19. The engineered T cell, composition, TCR protein, antibody-based receptor protein or nucleic acid molecule for use according to claim 16 or 17, wherein said liquid tumor is a myeloma, preferably a multiple myeloma, a leukemia, preferably an acute myeloid leukemia, or a lymphoma.

20. An isolated or purified peptide of human ropporin-1A (ROPN1) or human ropporin-1B (ROPN1B), which forms a complex with a human Major Histocompatibility Complex (MHC) molecule, preferably an HLA-A*02 molecule, wherein said peptide consists of the amino acid sequence of any one of SEQ ID NO:4, SEQ ID NO:43, SEQ ID NO:23, SEQ ID NO:56 and SEQ ID NO:24.

21. An engineered cell, preferably an engineered cancer cell, wherein said cell is engineered to express human ropporin-1A (ROPN1) and/or human ropporin-1B (ROPN1B).

22. A method of treating a subject suffering, or suspected of suffering, from a tumor, comprising the step of administering a therapeutically effective amount of an engineered T cell according to claim 1, a composition according to claim 12, a TCR protein or antibody-based receptor protein according to claim 13 or a nucleic acid molecule according to claim 14, to a subject in need thereof.


SEQUENCE LISTING (112 sequences)

SEQ ID NO: 1 - protein, 9 aa, Artificial Sequence
  Epitope 1 (MLN)
MLNYIEQEV

SEQ ID NO: 2 - protein, 9 aa, Artificial Sequence
  Epitope 2
FLALACSAL

SEQ ID NO: 3 - protein, 9 aa, Artificial Sequence
  Epitope 3
KTLKIVCEV

SEQ ID NO: 4 - protein, 9 aa, Artificial Sequence
  Epitope 4 (FLY-A)
FLYTYIAKV

SEQ ID NO: 5 - protein, 11 aa, Artificial Sequence
  Epitope 5
LIIRAEELAQM

SEQ ID NO: 6 - protein, 9 aa, Artificial Sequence
  Epitope 6
FQFLYTYIA

SEQ ID NO: 7 - protein, 9 aa, Artificial Sequence
  Epitope 7
ALACSALGV

SEQ ID NO: 8 - protein, 9 aa, Artificial Sequence
  Epitope 8
AQMWKVVNL

SEQ ID NO: 9 - protein, 9 aa, Artificial Sequence
  Epitope 9
KMLKEFAKA

SEQ ID NO: 10 - DNA, 822 bp, Artificial Sequence
  Epitope 1 (MLN)-specific TCR alpha chain non-modified nucleotide sequence
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagaggacggaggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 11 - protein, 273 aa, Artificial Sequence
  Epitope 1 (MLN)-specific TCR alpha chain amino acid sequence
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEDGGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - protein, 6 aa, Artificial Sequence
  Epitope 1 (MLN)-specific TCR alpha chain CDR1
DSSSTY

SEQ ID NO: 13 - protein, 7 aa, Artificial Sequence
  Epitope 1 (MLN)-specific TCR alpha chain CDR2
IFSNMDM

SEQ ID NO: 14 - protein, 11 aa, Artificial Sequence
  Epitope 1 (MLN)-specific TCR alpha chain CDR3
AEDGGGSYIPT

SEQ ID NO: 15 - DNA, 936 bp, Artificial Sequence
  Epitope 1 (MLN)-specific TCR beta chain non-modified nucleotide sequence
atggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgaatccggtatgcccaacaatcgattcttagctgaaaggactggagggacgtattctactctgaaggtgcagcctgcagaactggaggattctggagtttatttctgtgccagcagccccggccctgggcagaattcacccctccactttgggaatgggaccaggctcactgtgacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 16 - protein, 311 aa, Artificial Sequence
  Epitope 1 (MLN)-specific TCR beta chain amino acid sequence
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSPGPGQNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - protein, 5 aa, Artificial Sequence
  Epitope 1 (MLN)-specific TCR beta chain CDR1
SGHDN

SEQ ID NO: 18 - protein, 6 aa, Artificial Sequence
  Epitope 1 (MLN)-specific TCR beta chain CDR2
FVKESK

SEQ ID NO: 19 - protein, 13 aa, Artificial Sequence
  Epitope 1 (MLN)-specific TCR beta chain CDR3
ASSPGPGQNSPLH

SEQ ID NO: 20 - protein, 9 aa, Artificial Sequence
  Motif epitope 1 (MLN)
  MISC_FEATURE (6)..(6): X is any amino acid
  MISC_FEATURE (8)..(8): X is any amino acid
  MISC_FEATURE (9)..(9): X is any amino acid
MLNYIXQXX

SEQ ID NO: 21 - protein, 111 aa, Artificial Sequence
  TRAV and TRAJ domains of SEQ ID NO11
GEDVEQSLFLSVREGDSSVINCTYTDSSSTYLYWYKQEPGAGLQLLTYIFSNMDMKQDQRLTVLLNKKDKHLSLRIADTQTGDSAIYFCAEDGGGSYIPTFGRGTSLIVHP

SEQ ID NO: 22 - protein, 115 aa, Artificial Sequence
  TRBV, TRBD and TRBJ domains of SEQ ID NO16
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSPGPGQNSPLHFGNGTRLTVT

SEQ ID NO: 23 - protein, 9 aa, Artificial Sequence
  Epitope 10 (FLY-B)
FLYTYIAEV

SEQ ID NO: 24 - protein, 11 aa, Artificial Sequence
  Epitope 11 (EVI)
EVIGPDGLITV

SEQ ID NO: 25 - protein, 9 aa, Artificial Sequence
  Epitope 12
GLPRIPFST

SEQ ID NO: 26 - protein, 9 aa, Artificial Sequence
  Epitope 13
HVSRMLNYI

SEQ ID NO: 27 - protein, 9 aa, Artificial Sequence
  Epitope 14
RLIIRAEEL

SEQ ID NO: 28 - protein, 8 aa, Artificial Sequence
  Epitope 15
YIEVDGEI

SEQ ID NO: 29 - protein, 10 aa, Artificial Sequence
  Epitope 16
AELTPELLKI

SEQ ID NO: 30 - protein, 9 aa, Artificial Sequence
  Epitope 17
GVTITKTLK

SEQ ID NO: 31 - protein, 9 aa, Artificial Sequence
  Epitope 18
LPRIPFSTF

SEQ ID NO: 32 - protein, 9 aa, Artificial Sequence
  Epitope 19
SALGVTITK

SEQ ID NO: 33 - DNA, 825 bp, Artificial Sequence
  Epitope 4 (FLY-A)-specific TCR alpha chain non-modified nucleotide sequence
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacggggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 34 - protein, 274 aa, Artificial Sequence
  Epitope 4 (FLY-A)-specific TCR alpha chain amino acid sequence
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNGDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 35 - protein, 6 aa, Artificial Sequence
  Epitope 4 (FLY-A)-specific TCR alpha chain CDR1
DRGSQS

SEQ ID NO: 36 - protein, 6 aa, Artificial Sequence
  Epitope 4 (FLY-A)-specific TCR alpha chain CDR2
IYSNGD

SEQ ID NO: 37 - protein, 11 aa, Artificial Sequence
  Epitope 4 (FLY-A)-specific TCR alpha chain CDR3
AVNGDSSYKLI

SEQ ID NO: 38 - DNA, 927 bp, Artificial Sequence
  Epitope 4 (FLY-A)-specific TCR beta chain non-modified nucleotide sequence
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactccctaggggatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 39 - protein, 308 aa, Artificial Sequence
  Epitope 4 (FLY-A)-specific TCR beta chain amino acid sequence
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSLGDGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 40 - protein, 5 aa, Artificial Sequence
  Epitope 4 (FLY-A)-specific TCR beta chain CDR1
MNHEY

SEQ ID NO: 41 - protein, 6 aa, Artificial Sequence
  Epitope 4 (FLY-A)-specific TCR beta chain CDR2
SVGAGI

SEQ ID NO: 42 - protein, 11 aa, Artificial Sequence
  Epitope 4 (FLY-A)-specific TCR beta chain CDR3
ASSYSLGDGYT

SEQ ID NO: 43 - protein, 9 aa, Artificial Sequence
  Motif epitope 4 (FLY-A)
  misc_feature (1)..(2): Xaa can be any naturally occurring amino acid
  misc_feature (9)..(9): Xaa can be any naturally occurring amino acid
XXYTYIAKX

SEQ ID NO: 44 - protein, 111 aa, Artificial Sequence
  TRAV and TRAJ domains of SEQ ID NO34
QKEVEQNSGPLSVPEGAIASLNCTYSDRGSQSFFWYRQYSGKSPELIMSIYSNGDKEDGRFTAQLNKASQYVSLLIRDSQPSDSATYLCAVNGDSSYKLIFGSGTRLLVRP

SEQ ID NO: 45 - protein, 112 aa, Artificial Sequence
  TRBV, TRBD and TRBJ domains of SEQ ID NO39
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSLGDGYTFGSGTRLTVV

SEQ ID NO: 46 - DNA, 813 bp, Artificial Sequence
  Epitope 10 (FLY-B)-specific TCR alpha chain non-modified nucleotide sequence
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgctagggccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 47 - protein, 270 aa, Artificial Sequence
  Epitope 10 (FLY-B)-specific TCR alpha chain amino acid sequence
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDARARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 5 aa, Artificial Sequence
  Epitope 10 (FLY-B)-specific TCR alpha chain CDR1
TSINN

SEQ ID NO: 49 - protein, 7 aa, Artificial Sequence
  Epitope 10 (FLY-B)-specific TCR alpha chain CDR2
IRSNERE

SEQ ID NO: 50 - protein, 9 aa, Artificial Sequence
  Epitope 10 (FLY-B)-specific TCR alpha chain CDR3
ATDARARLM

SEQ ID NO: 51 - DNA, 948 bp, Artificial Sequence
  Epitope 10( FLY-B)-specific TCR beta chain non-modified nucleotide sequence
atggactcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttggggggggggacgaggcccctacctaattcacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 52 - protein, 315 aa, Artificial Sequence
  Epitope 10 (FLY-B)-specific TCR beta chain amino acid sequence
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGGGTRPLPNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 53 - protein, 5 aa, Artificial Sequence
  Epitope 10 (FLY-B)-specific TCR beta chain CDR1
SGHDY

SEQ ID NO: 54 - protein, 6 aa, Artificial Sequence
  Epitope 10 (FLY-B)-specific TCR beta chain CDR2
FNNNVP

SEQ ID NO: 55 - protein, 17 aa, Artificial Sequence
  Epitope 10 (FLY-B)-specific TCR beta chain CDR3
ASSLGGGTRPLPNSPLH

SEQ ID NO: 56 - protein, 9 aa, Artificial Sequence
  Motif Epitope 10 (FLY-B)
  misc_feature (1)..(1): Xaa can be any naturally occurring amino acid
  misc_feature (9)..(9): Xaa can be any naturally occurring amino acid
XLYTYIAEX

SEQ ID NO: 57 - protein, 109 aa, Artificial Sequence
  TRAV and TRAJ domains of SEQ ID NO47
SQQGEEDPQALSIQEGENATMNCSYKTSINNLQWYRQNSGRGLVHLILIRSNEREKHSGRLRVTLDTSKKSSSLLITASRAADTASYFCATDARARLMFGDGTQLVVKP

SEQ ID NO: 58 - protein, 119 aa, Artificial Sequence
  TRBV, TRBD and TRBJ domains of SEQ ID NO52
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGGGTRPLPNSPLHFGNGTRLTVT

SEQ ID NO: 59 - DNA, 822 bp, Artificial Sequence
  Epitope 11 (EVI)-specific TCR alpha chain non-modified nucleotide sequence
atgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgtgctgctcggacgggaggaggaaacaaactcacctttgggacaggcactcagctaaaagtggaactcaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 60 - protein, 273 aa, Artificial Sequence
  Epitope 11 (EVI)-specific TCR alpha chain amino acid sequence
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAARTGGGNKLTFGTGTQLKVELNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 61 - protein, 6 aa, Artificial Sequence
  Epitope 11 (EVI)-specific TCR alpha chain CDR1
SSVSVY

SEQ ID NO: 62 - protein, 8 aa, Artificial Sequence
  Epitope 11 (EVI)-specific TCR alpha chain CDR2
YLSGSTLV

SEQ ID NO: 63 - protein, 11 aa, Artificial Sequence
  Epitope 11 (EVI)-specific TCR alpha chain CDR3
AARTGGGNKLT

SEQ ID NO: 64 - DNA, 939 bp, Artificial Sequence
  Epitope 11 (EVI)-specific TCR beta chain non-modified nucleotide sequence
atgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaagaagggctagcgggagtaccccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctga

SEQ ID NO: 65 - protein, 312 aa, Artificial Sequence
  Epitope 11 (EVI)-specific TCR beta chain amino acid sequence
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQEGLAGVPQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 66 - protein, 5 aa, Artificial Sequence
  Epitope 11 (EVI)-specific TCR beta chain CDR1
MGHRA

SEQ ID NO: 67 - protein, 6 aa, Artificial Sequence
  Epitope 11 (EVI)-specific TCR beta chain CDR2
YSYEKL

SEQ ID NO: 68 - protein, 13 aa, Artificial Sequence
  Epitope 11 (EVI)-specific TCR beta chain CDR3
ASSQEGLAGVPQY

SEQ ID NO: 69 - protein, 113 aa, Artificial Sequence
  TRAV and TRAJ domains of SEQ ID NO60
AQSVTQLDSQVPVFEEAPVELRCNYSSSVSVYLFWYVQYPNQGLQLLLKYLSGSTLVKGINGFEAEFNKSQTSFHLRKPSVHISDTAEYFCAARTGGGNKLTFGTGTQLKVEL

SEQ ID NO: 70 - protein, 114 aa, Artificial Sequence
  TRBV, TRBD and TRBJ domains of SEQ ID NO65
DTEVTQTPKHLVMGMTNKKSLKCEQHMGHRAMYWYKQKAKKPPELMFVYSYEKLSINESVPSRFSPECPNSSLLNLHLHALQPEDSALYLCASSQEGLAGVPQYFGPGTRLLVL

SEQ ID NO: 71 - protein, 9 aa, Artificial Sequence
  epitope
YIAEVDGEI

SEQ ID NO: 72 - protein, 9 aa, Artificial Sequence
  epitope
HVSRMLNYI

SEQ ID NO: 73 - protein, 9 aa, Artificial Sequence
  eptiope
GLPRIPFST

SEQ ID NO: 74 - protein, 9 aa, Artificial Sequence
  epitope
RLIIRAEEL

SEQ ID NO: 75 - protein, 10 aa, Artificial Sequence
  epitope
AELTPELLKI

SEQ ID NO: 76 - protein, 11 aa, Artificial Sequence
  epitope
LIIRAEELAQM

SEQ ID NO: 77 - protein, 9 aa, Artificial Sequence
  epitope
YLEPGPVTA

SEQ ID NO: 78 - protein, 9 aa, Artificial Sequence
  epitope
SLLMWITQV

SEQ ID NO: 79 - protein, 9 aa, Artificial Sequence
  epitope
MLNYMEQEV

SEQ ID NO: 80 - protein, 9 aa, Artificial Sequence
  epitope
DLFNSVMNV

SEQ ID NO: 81 - protein, 9 aa, Artificial Sequence
  epitope
ELTPELLKI

SEQ ID NO: 82 - protein, 9 aa, Artificial Sequence
  epitope
LLKILHSQV

SEQ ID NO: 83 - protein, 9 aa, Artificial Sequence
  epitope
LTPELLKIL

SEQ ID NO: 84 - protein, 9 aa, Artificial Sequence
  epitope
TITKTLKIV

SEQ ID NO: 85 - protein, 9 aa, Artificial Sequence
  epitope
EISASHVSR

SEQ ID NO: 86 - protein, 9 aa, Artificial Sequence
  epitope
FTQNPRVWL

SEQ ID NO: 87 - protein, 9 aa, Artificial Sequence
  epitope
LPTDLFNSV

SEQ ID NO: 88 - protein, 9 aa, Artificial Sequence
  epitope
SPRIPFSTF

SEQ ID NO: 89 - protein, 13 aa, Artificial Sequence
  part of TCR sequence
CAEDGGGSYIPTF

SEQ ID NO: 90 - protein, 15 aa, Artificial Sequence
  part of TCR sequence
CASSPGPGQNSPLHF

SEQ ID NO: 91 - protein, 13 aa, Artificial Sequence
  part of TCR sequence
CAVNGDSSYKLIF

SEQ ID NO: 92 - protein, 13 aa, Artificial Sequence
  part of TCR sequence
CASSYSLGDGYTF

SEQ ID NO: 93 - protein, 15 aa, Artificial Sequence
  part of TCR sequence
CAYRASTSGTYKYIF

SEQ ID NO: 94 - protein, 13 aa, Artificial Sequence
  part of TCR sequence
CSALQGPSYEQYF

SEQ ID NO: 95 - protein, 15 aa, Artificial Sequence
  part of TCR sequence
CASSLQLAGVYEQYF

SEQ ID NO: 96 - protein, 16 aa, Artificial Sequence
  part of TCR sequence
CASSPGLAGVQETQYF

SEQ ID NO: 97 - protein, 16 aa, Artificial Sequence
  part of TCR sequence
CAVRDINSGGYQKVTF

SEQ ID NO: 98 - protein, 16 aa, Artificial Sequence
  part of TCR sequence
CASSPGPDGLENTIYF

SEQ ID NO: 99 - protein, 11 aa, Artificial Sequence
  part of TCR sequence
CATDARARLMF

SEQ ID NO: 100 - protein, 19 aa, Artificial Sequence
  part of TCR sequence
CASSLGGGTRPLPNSPLHF

SEQ ID NO: 101 - protein, 14 aa, Artificial Sequence
  part of TCR sequence
CAMSAPDSWGKLQF

SEQ ID NO: 102 - protein, 13 aa, Artificial Sequence
  part of TCR sequence
CASSPGDSQPQHF

SEQ ID NO: 103 - protein, 18 aa, Artificial Sequence
  part of TCR sequence
CATSSPSGGSRGRNEQFF

SEQ ID NO: 104 - protein, 13 aa, Artificial Sequence
  part of TCR sequence
  misc_feature (12)..(12): Xaa can be any naturally occurring amino acid
CAVEPGGSYIPXF

SEQ ID NO: 105 - protein, 13 aa, Artificial Sequence
  part of TCR sequence
CASSPGDSQPQHF

SEQ ID NO: 106 - protein, 13 aa, Artificial Sequence
  part of TCR sequence
CAGCPPNDYKLSF

SEQ ID NO: 107 - protein, 17 aa, Artificial Sequence
  part of TCR sequence
CASSNEFLQGSNEKLFF

SEQ ID NO: 108 - protein, 11 aa, Artificial Sequence
  part of TCR sequence
CAPPGDYKLSF

SEQ ID NO: 109 - protein, 13 aa, Artificial Sequence
  part of TCR sequence
CASSHILGIEQYF

SEQ ID NO: 110 - protein, 13 aa, Artificial Sequence
  part of TCR sequence
CAARTGGGNKLTF

SEQ ID NO: 111 - protein, 12 aa, Artificial Sequence
  part of TCR sequence
CASSLGGTEAFF

SEQ ID NO: 112 - protein, 15 aa, Artificial Sequence
  part of TCR sequence
CASSQEGLAGVPQYF